SEQUENCE LISTING (72 sequences)

SEQ ID NO: 1 - DNA, 1377 bp, danio rerio
atgggtgcgtgtatggccctgtgttcgctggcgagctgcgcgtcgtgtttgtgcggctctgctccgtgtcttttatcaggatgttgcccttcaacatacaactccactgtgacccgcttggccttctcctttttcctgctgctgggcactatagtgtccatcatcatgattctgccaggcatggagacacagctaaagaagattccaggattttgtgaaggaggctcttctatacctggatttgaagggaaggtgaactgtgatgtcattgttggctataaatcggtctatagaatgtgctttgccatggcctgtttcttcttccttttctccatcatcatgatccgtgtgagaagcagcaaggatccacgagctgcaattcaaaatggcttctggtttttcaagttcttgattctggtcggaattacagtgggagccttcttcattccagatggaatgttcaacacagtgtggtactacttcggtgtggttggctccttcatcttcattctgatccagctcattctgctggtcgattttgctcattcatggaaccaaaagtgggtggagaatgcagaggatgggaacagtaaatgctggtatgcagccctactcagtttcacattggtgcactatatctgtgcctttgctgcagttgtgttgttctatattttctacacccagccagatgactgcacagaaaacaaagtcttcatcagtcttaacctgatcttttgtatcattgtgtctgtggtggctatccttccaaaagtgcaggaagctcagccaagctctggtctcctccaggcgtccctcatctctctctacaccatgtatctcacatggtctgccatgagcaacaaccccaaccgtaagtgcaaccccagtctgttaagtatagtcaaaggccaatccgcagcacccactcctaccagcacccctggtgtctttacacaatggtgggatgcccagagcgttgtgggtttggtcatcttcctcctgtgcacactgtatgccagcattcgttcctcaaacaacagccaagtgaataagctcatgcagacagaggaggttcagggtctggcgagttctgatgccaatgatgccatctctgaggatggcgtgagacgagctgtggataacgaagaggacggtgtcacatacggctactctttcttccatttctctctctttctcgcctctctctacatcatgatgaccctcaccaactggtaccaacctgaaactgactatgcagccatgacgaccactatgccatcagtttgggtgaagatttgttccagttggctgggattgggtctgtacctctggactcttgttgctccactagtcctgactgaccgggacttcaactga

SEQ ID NO: 2 - DNA, 1893 bp, danio rerio
gctgctttagtttcggtgtgttttcagacattatgggtgcgtgtatggccctgtgttcgctggcgagctgcgcgtcgtgtttgtgcggctctgctccgtgtcttttatcaggatgttgcccttcaacatacaactccactgtgacccgcttggccttctcctttttcctgctgctgggcactatagtgtccatcatcatgattctgccaggcatggagacacagctaaagaagattccaggattttgtgaaggaggctcttctatacctggatttgaagggaaggtgaactgtgatgtcattgttggctataaatcggtctatagaatgtgctttgccatggcctgtttcttcttccttttctccatcatcatgatccgtgtgagaagcagcaaggatccacgagctgcaattcaaaatggcttctggtttttcaagttcttgattctggtcggaattacagtgggagccttcttcattccagatggaatgttcaacacagtgtggtactacttcggtgtggttggctccttcatcttcattctgatccagctcattctgctggtcgattttgctcattcatggaaccaaaagtgggtggagaatgcagaggatgggaacagtaaatgctggtatgcagccctactcagtttcacattggtgcactatatctgtgcctttgctgcagttgtgttgttctatattttctacacccagccagatgactgcacagaaaacaaagtcttcatcagtcttaacctgatcttttgtatcattgtgtctgtggtggctatccttccaaaagtgcaggaagctcagccaagctctggtctcctccaggcgtccctcatctctctctacaccatgtatctcacatggtctgccatgagcaacaaccccaaccgtaagtgcaaccccagtctgttaagtatagtcaaaggccaatccgcagcacccactcctaccagcacccctggtgtctttacacaatggtgggatgcccagagcgttgtgggtttggtcatcttcctcctgtgcacactgtatgccagcattcgttcctcaaacaacagccaagtgaataagctcatgcagacagaggaggttcagggtctggcgagttctgatgccaatgatgccatctctgaggatggcgtgagacgagctgtggataacgaagaggacggtgtcacatacggctactctttcttccatttctctctctttctcgcctctctctacatcatgatgaccctcaccaactggtaccaacctgaaactgactatgcagccatgacgaccactatgccatcagtttgggtgaagatttgttccagttggctgggattgggtctgtacctctggactcttgttgctccactagtcctgactgaccgggacttcaactgaacacctgtaagatactgcaaacaatctaccttttaaaccaaatcatatgcagtcacagaaattcttgttcatctataaaatgacttgagctttcaaactcaattctcgcacacttaaccttatgcacactttgttacatagttattcacttttagaagagatccgtaaatgcacttcagaaagcttcaattttgttaatgctgcattaataagacttctgtttaaaggtttgttttcttctttcatactttgatgtatcatgcatatgttgaatttacaaaagctgatgtataagacagttgaactatttctaaatattttttattatcatgtatatgtgttttgttgcaaaatgcactacatttaatcaaatcaaagttgaatgtaccggaaaacaattgaaaagtgccttatctgtattgagattttaatttgcataaagacagctttattttaggagccttatgataacactcctcctgaa

SEQ ID NO: 3 - protein, 458 aa, danio rerio
MGACMALCSLASCASCLCGSAPCLLSGCCPSTYNSTVTRLAFSFFLLLGTIVSIIMILPGMETQLKKIPGFCEGGSSIPGFEGKVNCDVIVGYKSVYRMCFAMACFFFLFSIIMIRVRSSKDPRAAIQNGFWFFKFLILVGITVGAFFIPDGMFNTVWYYFGVVGSFIFILIQLILLVDFAHSWNQKWVENAEDGNSKCWYAALLSFTLVHYICAFAAVVLFYIFYTQPDDCTENKVFISLNLIFCIIVSVVAILPKVQEAQPSSGLLQASLISLYTMYLTWSAMSNNPNRKCNPSLLSIVKGQSAAPTPTSTPGVFTQWWDAQSVVGLVIFLLCTLYASIRSSNNSQVNKLMQTEEVQGLASSDANDAISEDGVRRAVDNEEDGVTYGYSFFHFSLFLASLYIMMTLTNWYQPETDYAAMTTTMPSVWVKICSSWLGLGLYLWTLVAPLVLTDRDFN

SEQ ID NO: 4 - DNA, 1419 bp, mus musculus
atgggggccgtcctcgcggtcttctccctcgccagctgggtcccgtgcctctgtagtggtgcatcatgtctgctgtgcagttgctgtcccatcagtaagaattccactgtaactcggctcatctacgcttttatcctcttccttggcactattgtgtcttgcatcatgatgacagaaggcatacaaactcaactgaagaagattcctggattctgtgaaggaggatttcaaatcaagatggttgatacaaaggcagagaaagattgtgacgtgctggtcggttttaaagctgtgtatcggatcaactttgctgtggccatctttttctttgccttctttttgctcatgttaaaagttaaaacaagtaaagatcccagagcagcagtgcacaacgggttttggttcttcaaaatcgctgccattattggtatcatgattggatctttctacatccctgggggcagttttactgaagtctggtttgttgctggaatgttgggggcctctttcttcattatcatccagctggtgctcttggtagacatggctcactcttggaatgaattatgggtaaatcgaatggaggaaggaaacccaaggctctggtatgctgccttgctgtcctttacaagcctcttttacatcctctccatcgtctttgctgcgctgctctacgtcttctacaccaagcctgacgactgcacagaaaacaaggtcttcatcagcctcaacctgattttttgtgttgcagtttctattgtgtccatcctccctaaagttcaggaacatcagcctcgctctggcctcctgcagtcctccatcatcactctgtacaccctttacctcacgtggtcagccatgaccaatgaacctgagcggtcctgcaatccctccttaatgagcatcatcacacacttaacttcaccaactgtgtctcctgcaaattcaactactcttgctcctgcctatcgtccgccgtcacagagtgggcactttatgaatttggatgatatttggggactgattatctttgttttctgccttatatattctagcttccgtacttcgagcaacagccaagttaacaagctgaccctctctgggagtgacagtgttatccttggtgataccaccaatggagccaatgatgaagaggatggacagccacggagggctgtagacaatgagaaggagggggtgcagtatagctactcctttttccacttgatgctctgctgtgcctccttgtacatcatgatgaccataaccagctggtacagccctgatgccaaattccagaaggtatccagcaagtggctagctgtgtggttcaaaatgggctccagctggttgtgcctcctcctttacctctggactcttgtggctcccctggtcctcacaggtcgggacttcagctga

SEQ ID NO: 5 - DNA, 2232 bp, mus musculus
cgctcggagcggtcggtctcggccccggcgtcaccatgggggccgtcctcgcggtcttctccctcgccagctgggtcccgtgcctctgtagtggtgcatcatgtctgctgtgcagttgctgtcccatcagtaagaattccactgtaactcggctcatctacgcttttatcctcttccttggcactattgtgtcttgcatcatgatgacagaaggcatacaaactcaactgaagaagattcctggattctgtgaaggaggatttcaaatcaagatggttgatacaaaggcagagaaagattgtgacgtgctggtcggttttaaagctgtgtatcggatcaactttgctgtggccatctttttctttgccttctttttgctcatgttaaaagttaaaacaagtaaagatcccagagcagcagtgcacaacgggttttggttcttcaaaatcgctgccattattggtatcatgattggatctttctacatccctgggggcagttttactgaagtctggtttgttgctggaatgttgggggcctctttcttcattatcatccagctggtgctcttggtagacatggctcactcttggaatgaattatgggtaaatcgaatggaggaaggaaacccaaggctctggtatgctgccttgctgtcctttacaagcctcttttacatcctctccatcgtctttgctgcgctgctctacgtcttctacaccaagcctgacgactgcacagaaaacaaggtcttcatcagcctcaacctgattttttgtgttgcagtttctattgtgtccatcctccctaaagttcaggaacatcagcctcgctctggcctcctgcagtcctccatcatcactctgtacaccctttacctcacgtggtcagccatgaccaatgaacctgagcggtcctgcaatccctccttaatgagcatcatcacacacttaacttcaccaactgtgtctcctgcaaattcaactactcttgctcctgcctatcgtccgccgtcacagagtgggcactttatgaatttggatgatatttggggactgattatctttgttttctgccttatatattctagcttccgtacttcgagcaacagccaagttaacaagctgaccctctctgggagtgacagtgttatccttggtgataccaccaatggagccaatgatgaagaggatggacagccacggagggctgtagacaatgagaaggagggggtgcagtatagctactcctttttccacttgatgctctgctgtgcctccttgtacatcatgatgaccataaccagctggtacagccctgatgccaaattccagaaggtatccagcaagtggctagctgtgtggttcaaaatgggctccagctggttgtgcctcctcctttacctctggactcttgtggctcccctggtcctcacaggtcgggacttcagctgagctcagtgtgtcaaggacactgataaagctgaccagagtctccttttctgaaaatgcatatccattttgcgtttcatcaacgagactattaagtgaacgctttgcagatttggctgtattcaggtttatatcaaaaggcaagattgagtaatgcttgatgcagaatctgagctttcatatatatatatatatatatatatatatatacacacacacacacacacatatatatgtttatttgtaaggctatagcacaaagggaacatttttgtgttttaacatgaactacagctgtgctgtgaagagaattctttataaagacctgtagattcctacaactttggtttaagttttaagttagaagattgttggatatttaaggctatttttaatttctattacagtctccttaaaaaccaaaaaggaatgcattaatccacatttcccttcttcagaggtgtagtgtcctggctcttggcaaggaattatgtatttaggtcagtccccagaaatgcagcgctcatacagctgagagaaggctattattgagttcctttacttactttttatactacactgatgctgcttgatagaagtctgtgggctttgtcagatatgtcacccaagtaaatgctttgtagatctgattaaaatgaaaagctcacttgagaaacactgcagagttatgtaatgatcttgttgtgagtgtgtgaaagtcaaaggcatgtcagtttattacatttgcaacataaaagtacttaattaaaatagaaaaaaa

SEQ ID NO: 6 - protein, 472 aa, mus musculus
MGAVLAVFSLASWVPCLCSGASCLLCSCCPISKNSTVTRLIYAFILFLGTIVSCIMMTEGIQTQLKKIPGFCEGGFQIKMVDTKAEKDCDVLVGFKAVYRINFAVAIFFFAFFLLMLKVKTSKDPRAAVHNGFWFFKIAAIIGIMIGSFYIPGGSFTEVWFVAGMLGASFFIIIQLVLLVDMAHSWNELWVNRMEEGNPRLWYAALLSFTSLFYILSIVFAALLYVFYTKPDDCTENKVFISLNLIFCVAVSIVSILPKVQEHQPRSGLLQSSIITLYTLYLTWSAMTNEPERSCNPSLMSIITHLTSPTVSPANSTTLAPAYRPPSQSGHFMNLDDIWGLIIFVFCLIYSSFRTSSNSQVNKLTLSGSDSVILGDTTNGANDEEDGQPRRAVDNEKEGVQYSYSFFHLMLCCASLYIMMTITSWYSPDAKFQKVSSKWLAVWFKMGSSWLCLLLYLWTLVAPLVLTGRDFS

SEQ ID NO: 7 - DNA, 1422 bp, homo sapiens
atgggggctgtgctgggtgtcttctccctcgccagctgggttccatgcctctgcagcggtgcctcatgtttgctgtgtagttgctgtcctaacagtaagaattccacggtgactcgcctcatttatgctttcattctcctcctgagcactgtcgtatcctatatcatgcagagaaaagagatggaaacttacttgaagaagattcctggattttgtgaagggggatttaaaatccatgaggctgatataaatgcagataaagattgtgatgtgctggttggttataaagctgtgtatcggatcagctttgccatggccatctttttctttgtcttttctctgctcatgttcaaagtaaaaacaagtaaagatctccgagcggcagtacacaatgggttttggttcttcaaaattgctgcccttattggaatcatggttggctctttctacatccctgggggctatttcagctcagtctggtttgttgttggcatgataggggccgccctcttcatcctcattcagctggtgctgctggtagattttgctcattcttggaatgaatcatgggtaaatcgaatggaagaaggaaacccaaggttgtggtatgctgctttactgtctttcacaagcgccttttatatcctgtcaatcatctgtgtcgggctgctctatacatattacaccaaaccagatggctgcacagaaaacaagttcttcatcagtattaacctgatcctttgcgttgtggcttctattatatcgatccacccaaaaattcaggaacaccagcctcgctccggcctcttgcagtcctccctcatcaccctctacactatgtacctcacctggtcagccatgtccaatgaacctgatcgttcctgcaatcccaacctgatgagctttattacacgcataactgcaccaaccctggctcctggaaattcaactgctgtggtccctacccctactccaccatcaaagagtgggtctttactggattcagataattttattggactgtttgtctttgttctctgcctcttgtattctagcatccgcacttccactaatagccaagtagacaagctgaccctgtcagggagtgacagcgtcatccttggtgatacaactaccagtggtgccagtgatgaagaagatggacagcctcggcgggctgtggacaacgagaaagagggagtgcagtatagctactccttattccacctcatgctctgcttggcttccttgtacatcatgatgaccctgaccagctggtacagccctgatgcaaagtttcagagcatgaccagcaagtggccagctgtgtgggtcaagatcagctccagctgggtctgcctcctgctttacgtctggacccttgtggctccacttgtcctcaccagtcgggacttcagctga

SEQ ID NO: 8 - DNA, 1892 bp, homo sapiens
ggcacgagctcagctggcagttaccaccgtgttagaaagcagcctcaggaccggccacctccatcactggcgtcaccatgggggctgtgctgggtgtcttctccctcgccagctgggttccatgcctctgcagcggtgcctcatgtttgctgtgtagttgctgtcctaacagtaagaattccacggtgactcgcctcatttatgctttcattctcctcctgagcactgtcgtatcctatatcatgcagagaaaagagatggaaacttacttgaagaagattcctggattttgtgaagggggatttaaaatccatgaggctgatataaatgcagataaagattgtgatgtgctggttggttataaagctgtgtatcggatcagctttgccatggccatctttttctttgtcttttctctgctcatgttcaaagtaaaaacaagtaaagatctccgagcggcagtacacaatgggttttggttcttcaaaattgctgcccttattggaatcatggttggctctttctacatccctgggggctatttcagctcagtctggtttgttgttggcatgataggggccgccctcttcatcctcattcagctggtgctgctggtagattttgctcattcttggaatgaatcatgggtaaatcgaatggaagaaggaaacccaaggttgtggtatgctgctttactgtctttcacaagcgccttttatatcctgtcaatcatctgtgtcgggctgctctatacatattacaccaaaccagatggctgcacagaaaacaagttcttcatcagtattaacctgatcctttgcgttgtggcttctattatatcgatccacccaaaaattcaggaacaccagcctcgctccggcctcttgcagtcctccctcatcaccctctacactatgtacctcacctggtcagccatgtccaatgaacctgatcgttcctgcaatcccaacctgatgagctttattacacgcataactgcaccaaccctggctcctggaaattcaactgctgtggtccctacccctactccaccatcaaagagtgggtctttactggattcagataattttattggactgtttgtctttgttctctgcctcttgtattctagcatccgcacttccactaatagccaagtagacaagctgaccctgtcagggagtgacagcgtcatccttggtgatacaactaccagtggtgccagtgatgaagaagatggacagcctcggcgggctgtggacaacgagaaagagggagtgcagtatagctactccttattccacctcatgctctgcttggcttccttgtacatcatgatgaccctgaccagctggtacagccctgatgcaaagtttcagagcatgaccagcaagtggccagctgtgtgggtcaagatcagctccagctgggtctgcctcctgctttacgtctggacccttgtggctccacttgtcctcaccagtcgggacttcagctgaacctctgagtgccaaggacaccactggaactcacaaaggtctccttcaccgaaaacccatataccttttaagtttgtttcaactaaaatattaagtgaatgctttgcaagtttgactgtatgcaggtttatatcagaaggtgagattgaataatgcttgatgcagaatcgaaacttctcatttatctgtatattatgtttacttctaaggatatagcacaaagggaacattttttgtttaaagtgaactacagctgtgctgtgaagagagttctttataaagcctgtaggttcttttaactttggtttaaaatgtaagataggaaaatgttggatatttgaggccatgcttaatatatttatattgcagtatcctttaaaagcaaaaaaaaaa

SEQ ID NO: 9 - protein, 473 aa, homo sapiens
MGAVLGVFSLASWVPCLCSGASCLLCSCCPNSKNSTVTRLIYAFILLLSTVVSYIMQRKEMETYLKKIPGFCEGGFKIHEADINADKDCDVLVGYKAVYRISFAMAIFFFVFSLLMFKVKTSKDLRAAVHNGFWFFKIAALIGIMVGSFYIPGGYFSSVWFVVGMIGAALFILIQLVLLVDFAHSWNESWVNRMEEGNPRLWYAALLSFTSAFYILSIICVGLLYTYYTKPDGCTENKFFISINLILCVVASIISIHPKIQEHQPRSGLLQSSLITLYTMYLTWSAMSNEPDRSCNPNLMSFITRITAPTLAPGNSTAVVPTPTPPSKSGSLLDSDNFIGLFVFVLCLLYSSIRTSTNSQVDKLTLSGSDSVILGDTTTSGASDEEDGQPRRAVDNEKEGVQYSYSLFHLMLCLASLYIMMTLTSWYSPDAKFQSMTSKWPAVWVKISSSWVCLLLYVWTLVAPLVLTSRDFS

SEQ ID NO: 10 - DNA, 786 bp, danio rerio
atggctgagcccgagctcctcctggactccaacatccgtctttgggttgtgctgccaattgtatttattacttttctcgttggtgtgatccgccattatgtctcaattctgttacaaagtgataagaagcttacattagagcaggtttcagacagccaggttcttatcaggagcagagttttacgagaaaatggcaaatacatccctaaacaatctttcttgatgagaaaattctacttcaataaccaagaagatgggttttttaagaagactaagagaaaggtggtcccaccgtctccgatgactgatccgagcatgttgaccgatatgatgaaaggcaacgtgacaaatgtcctgcctatgattcttattggtggttggatcaactggaccttctctgggtttgttacaacaaaagtaccgttccctctcacactccgcttcaagccaatgctgcagcagggaattgagttgctctctctagatgcctcctgggtgagctcagcatcttggtatttcctgaatgtctttggtctcaggagtatgtattccctgattcttggacaggataatggtgcagatcagtctcgcattatgcaggaacagatgagtggtgctgcaatggcaatgccagctgatacaaacaaagcattcaaggctgaatgggaggcgctggagttgactgatcaccaatgggctttggagaatgttgaggaggatctaatgagtaaggacttggatttgtctgggatgttcagcaaagacttgccaacgggcatcttttaa

SEQ ID NO: 11 - DNA, 2515 bp, danio rerio
tggctctatggagtcgcatcctgattcgagtcgttatcggtcttctcacagataaaccgtcatttgaatacgagagcagggtggtgaaattccatcagcatctcttcctaaaaagcatggctgagcccgagctcctcctggactccaacatccgtctttgggttgtgctgccaattgtatttattacttttctcgttggtgtgatccgccattatgtctcaattctgttacaaagtgataagaagcttacattagagcaggtttcagacagccaggttcttatcaggagcagagttttacgagaaaatggcaaatacatccctaaacaatctttcttgatgagaaaattctacttcaataaccaagaagatgggttttttaagaagactaagagaaaggtggtcccaccgtctccgatgactgatccgagcatgttgaccgatatgatgaaaggcaacgtgacaaatgtcctgcctatgattcttattggtggttggatcaactggaccttctctgggtttgttacaacaaaagtaccgttccctctcacactccgcttcaagccaatgctgcagcagggaattgagttgctctctctagatgcctcctgggtgagctcagcatcttggtatttcctgaatgtctttggtctcaggagtatgtattccctgattcttggacaggataatggtgcagatcagtctcgcattatgcaggaacagatgagtggtgctgcaatggcaatgccagctgatacaaacaaagcattcaaggctgaatgggaggcgctggagttgactgatcaccaatgggctttggagaatgttgaggaggatctaatgagtaaggacttggatttgtctgggatgttcagcaaagacttgccaacgggcatcttttaagtgcctgttcactactgagaggtgttggcagacctcactgagttaaaaagagcgaattgtcggactggttttggagtgaccatacgcttccagtcagagttgtctttctagaaagtgtcataaacatcattgctgacatatcacattttgtgtcatatgcatgcaccgttccctccactgctgattttttgctcagacttgagcattatagtttgatcaagcctagagcttaatggattttacagattacagtgaaaaccatagtgtagctgttttttttaacattcctccattaatattaatattttttatttgatttctcttaaacaaatttatattaattttagttgttttaaaagcccttttcttggtcttatttcactaggacagtgtgattgtttggaccaaacagacaaaaaggaagttttaggaagtaaagtggtttaaaatggagcagtccataatttagtttgtttcataaatagtattttcaaaatgtaatagacttttagatgctttatgatgccaataacacaccacacactaagtatattgtttttaaaaacaaacaatttggaattaagatcattgagtagattttcctcattagaaattcatatttattgagttgttagcttgtaatatatatatttttggacttttattaaacatctttgttttttttttttaatacatttttccacatcacctttcatggatgtgcacatttttaatacaaaaacagtcatgcactgttctgttttctgtatttattttttaactagcttttctaccactgctgattttatttggtccatttgtgcagattcattgctttcaagaaacaagtgtagtcttcgtaatttccagcaaaatttaattatttgctctgccactttgaaaacttcaactggggcctgtaacataaagccagattagatgtctagccagttaaatttctgtttagtttgcaccagtcttgggttttaggtgacgtgacggcaacttgaatttaatcactcttgttgtggtattatggagtagcttagttgagctaggtggcatggtggctcagtggttagcactgtcgcgtcactacaagaaggttgctagtttggttccagctggcatttctgtgtgaagtttgcatgttctccccatgttggtgtgggttttctccaggagctccagtttcccccacagtccaaagacatgcgctataggtgtactgctcttaattggctgtagtgtggtgtttctcagtactgggttggggctggaagggcatccgctgcgtaaaacatgctgtgataaataagggcctaaactgaaagaaaatgaatgaatgaatgaatgagctgagctacctttatggtatataaaaaacagaattggtgcaaactattctggcttcacggtgcaggcccctgatcctccctgcagtggcttgttaaaaaatacctacacaacaatataatcatgattaaactaaaatgttgatcatttatattaaagaaaacaaaaatgtacatttttggacctttgaataaaacgctatttatcgaaaccagtacagttttgtaaacatgttaataaaaccatcatgcaacata

SEQ ID NO: 12 - protein, 261 aa, danio rerio
MAEPELLLDSNIRLWVVLPIVFITFLVGVIRHYVSILLQSDKKLTLEQVSDSQVLIRSRVLRENGKYIPKQSFLMRKFYFNNQEDGFFKKTKRKVVPPSPMTDPSMLTDMMKGNVTNVLPMILIGGWINWTFSGFVTTKVPFPLTLRFKPMLQQGIELLSLDASWVSSASWYFLNVFGLRSMYSLILGQDNGADQSRIMQEQMSGAAMAMPADTNKAFKAEWEALELTDHQWALENVEEDLMSKDLDLSGMFSKDLPTGIF

SEQ ID NO: 13 - DNA, 786 bp, mus musculus
atggcggggcccgagctgctgcttgactccaacatccgcctctgggtggtcctgcccatcgttatcatcactttcttcgtgggcatgatccgccactacgtgtcaatcctactgcagagcgacaagaagctcacccaggaacaagtgtctgacagtcaggtcctaattcgaagcagagtcctcagggaaaatggaaaatacattcccaagcagtctttcttaacacgaaaatattacttcaacaacccagaggatggatttttcaaaaaaacaaaaaggaaggttgtgccaccttcccccatgacagaccccaccatgctcacagacatgatgaaagggaatgtcacaaatgtcctcccaatgattcttatcggcggatggatcaacatgacgttttcaggctttgtcacaactaaggtcccgtttccactgacacttcgcttcaagcctatgcttcagcaaggaatagagctgctcacactagacgcatcctgggtgagttctgcatcctggtacttcctcaatgtgtttgggctccggagcatttactctctaatcctgggccaagataacgccgccgaccagtcacgaatgatgcaggagcagatgacaggagcagcgatggccatgcctgcagacaccaacaaagctttcaagacagagtgggaagctttggaactgacagatcaccagtgggcgctcgatgatgtggaagaagaactcatggccagagacctccactttgaaggcatgttcaaaaaggaactacagacgtccatattctaa

SEQ ID NO: 14 - DNA, 1877 bp, mus musculus
ctggaaacagtgcgcggagaaagctaggctgccccagattccacgccagcgaactctcagcgaagatggcggggcccgagctgctgcttgactccaacatccgcctctgggtggtcctgcccatcgttatcatcactttcttcgtgggcatgatccgccactacgtgtcaatcctactgcagagcgacaagaagctcacccaggaacaagtgtctgacagtcaggtcctaattcgaagcagagtcctcagggaaaatggaaaatacattcccaagcagtctttcttaacacgaaaatattacttcaacaacccagaggatggatttttcaaaaaaacaaaaaggaaggttgtgccaccttcccccatgacagaccccaccatgctcacagacatgatgaaagggaatgtcacaaatgtcctcccaatgattcttatcggcggatggatcaacatgacgttttcaggctttgtcacaactaaggtcccgtttccactgacacttcgcttcaagcctatgcttcagcaaggaatagagctgctcacactagacgcatcctgggtgagttctgcatcctggtacttcctcaatgtgtttgggctccggagcatttactctctaatcctgggccaagataacgccgccgaccagtcacgaatgatgcaggagcagatgacaggagcagcgatggccatgcctgcagacaccaacaaagctttcaagacagagtgggaagctttggaactgacagatcaccagtgggcgctcgatgatgtggaagaagaactcatggccagagacctccactttgaaggcatgttcaaaaaggaactacagacgtccatattctaaccacatgcggggtcagctgtgtccggaacttgcagtagcacttaaccttgtaacttccgtggagctggagcctctgagaataaaaaggagggtgcaggggctggcgggtgcagcaaggctcattcttgtctgagctgggttcccctttatgttggaaactagaggaaaaggagttgtgggtgactgctgtcttaaagtttgtgattgttccttcaggtgactctagtaaggacgtcagagaaaggggagaccccatgctactgagaatagctcaacccttagcaaaaactccgtgcttggaaagagcattgtcagttatagcagttactatgttcggtcagcctgacttccagccacagtaaactcctgttttcttaggatccaaacaccctgcattttaccttgaatttcttgtttgtatttttaactttctttacacacgtaatatacttttctctaccacaatttagaaactatggctgagcagcacagcgctccccgcactgttcacttgagcctacaaaggacgcagagaactggtgtggcccaacattgctgaaataaactctttgcagagtggatccccagggccggggcagactcagcaaacttttcatatctcttcatacttagtccagtgaaaacaggtgaccctgaggcacagccagactctcaagtgcctttgggctcatcagagacaggttacatatacaagttcttgattggaacttgaggaaatatcaactctatcggccagtcaatggtgctgtgctgtaaatggctactcatttgaacgaatgacctcatcccagttcctctgggcaggagagaacttaccactgttggggcaagaggacagtttctggtggatatgtagattaaggtgcccaggagtcctctgaccagggaagtgttagggtgccatttctgtaccaaaagctgggtgtgttgcatgttaagcactgtagccaagaacaagcgactcttgactcttaattaaacagccctgttttctctttgcctgtg

SEQ ID NO: 15 - protein, 261 aa, mus musculus
MAGPELLLDSNIRLWVVLPIVIITFFVGMIRHYVSILLQSDKKLTQEQVSDSQVLIRSRVLRENGKYIPKQSFLTRKYYFNNPEDGFFKKTKRKVVPPSPMTDPTMLTDMMKGNVTNVLPMILIGGWINMTFSGFVTTKVPFPLTLRFKPMLQQGIELLTLDASWVSSASWYFLNVFGLRSIYSLILGQDNAADQSRMMQEQMTGAAMAMPADTNKAFKTEWEALELTDHQWALDDVEEELMARDLHFEGMFKKELQTSIF

SEQ ID NO: 16 - DNA, 786 bp, homo sapiens
atggcagggccagaactgttgctcgactccaacatccgcctctgggtggtcctacccatcgttatcatcactttcttcgtaggcatgatccgccactacgtgtccatcctgctgcagagcgacaagaagctcacccaggaacaagtatctgacagtcaagtcctaattcgaagcagagtcctcagggaaaatggaaaatacattcccaaacagtctttcttgacacgaaaatattatttcaacaacccagaggatggatttttcaaaaaaactaaacggaaggtagtgccaccttctcctatgactgatcctactatgttgacagacatgatgaaagggaatgtaacaaatgtcctccctatgattcttattggtggatggatcaacatgacattctcaggctttgtcacaaccaaggtcccatttccactgaccctccgttttaagcctatgttacagcaaggaatcgagctactcacattagatgcatcctgggtgagttctgcatcctggtacttcctcaatgtatttgggcttcggagcatttactctctgattctgggccaagataatgccgctgaccaatcacgaatgatgcaggagcagatgacgggagcagccatggccatgcccgcagacacaaacaaagctttcaagacagagtgggaagctttggagctgacggatcaccagtgggcactagatgatgtcgaagaagagctcatggccaaagacctccacttcgaaggcatgttcaaaaaggaattacagacctctattttttga

SEQ ID NO: 17 - DNA, 1109 bp, homo sapiens
actggaagaccaggcagcccagctgaaggcagtaagctcggctcacagtcgcaggagagttctggggtacacgggcaaaggggcttgagaaggcccggaggcgaagccgaagagaagcaactgtgccccggagaagagaagctcgcccattccagactgggaaccagctttcagtgaagatggcagggccagaactgttgctcgactccaacatccgcctctgggtggtcctacccatcgttatcatcactttcttcgtaggcatgatccgccactacgtgtccatcctgctgcagagcgacaagaagctcacccaggaacaagtatctgacagtcaagtcctaattcgaagcagagtcctcagggaaaatggaaaatacattcccaaacagtctttcttgacacgaaaatattatttcaacaacccagaggatggatttttcaaaaaaactaaacggaaggtagtgccaccttctcctatgactgatcctactatgttgacagacatgatgaaagggaatgtaacaaatgtcctccctatgattcttattggtggatggatcaacatgacattctcaggctttgtcacaaccaaggtcccatttccactgaccctccgttttaagcctatgttacagcaaggaatcgagctactcacattagatgcatcctgggtgagttctgcatcctggtacttcctcaatgtatttgggcttcggagcatttactctctgattctgggccaagataatgccgctgaccaatcacgaatgatgcaggagcagatgacgggagcagccatggccatgcccgcagacacaaacaaagctttcaagacagagtgggaagctttggagctgacggatcaccagtgggcactagatgatgtcgaagaagagctcatggccaaagacctccacttcgaaggcatgttcaaaaaggaattacagacctctattttttgaagaccgagcagggattagctgtgtcaggaacttggagttgcacttaaccttgtaactttgtttggagctggcacctcttgaaataaaaaggaggatgcacgagctggcaggcatgcaaaaaaaaaaaaaaaaaaaaaaaaaaaa

SEQ ID NO: 18 - protein, 261 aa, home sapiens
MAGPELLLDSNIRLWVVLPIVIITFFVGMIRHYVSILLQSDKKLTQEQVSDSQVLIRSRVLRENGKYIPKQSFLTRKYYFNNPEDGFFKKTKRKVVPPSPMTDPTMLTDMMKGNVTNVLPMILIGGWINMTFSGFVTTKVPFPLTLRFKPMLQQGIELLTLDASWVSSASWYFLNVFGLRSIYSLILGQDNAADQSRMMQEQMTGAAMAMPADTNKAFKTEWEALELTDHQWALDDVEEELMAKDLHFEGMFKKELQTSIF

SEQ ID NO: 19 - DNA, 1740 bp, danio rerio
atgccgaaaacgatcagtgttcgtgtgactacgatggatgccgagctggagtttgccatccaacccagcaccacagggaaacagttatttgaccaggtggtgaaaaccattgggctgagggaggtgtggttttttggtcttcagtatcaggacaccaaaggtttttccacctggcttaaactcaacaagaaggttacggctcaagacgtccgcaaggagagtcctttgctgtttaaattcagagcaaagttttacccagaagacgtttctgaggaactgattcaggaggccactcaaagactcttcttccttcaggttaaagagggcatcctgaatgacgatatctactgccctcctgagactgcggttttgctggcctcttatgctgtgcaggccaaatatgcagactacaacaaagatgcccacactcctggatacttgtccaatgagaagctgctcccacagagagttctggagcagcacaagctgaataaggagcaatgggaggaaagaattcaggtctggcacgaggagcacaagggcatgttgagagaggactcaatgatggagtatctgaagatagctcaggatctggagatgtatggtgtgaattacttcagtattaagaataagaagggatcagagctgtggcttggtgtggatgctctgggactcaacatctacgagcagaatgacaagatgacacctaaaattggtttcccctggagtgagatcaggaacatttctttcaacgataagaaatttgtcatcaaacccattgataaaaaagccccggactttgtgttctatgcccagcgtttacgcattaataagaggattctggctctgtgcatggggaaccatgagctttatatgagacgccgtaaaccagacaccatcgaggtccagcagatgaaggcacaggccaaggaggaaaagaaccacaagaagatggaaagggcattgttggaggatgaacgtaaaaagagagagcaggccgaaaaggagaaagagaagattgaaaaagaaaaagaggagctgatggagagactcagagtaatcgaggagcagacaagaaaagctcagcaagagctggaggagcagacccgcaaggcgctggagttagagcaggagcgtaaacgtgctcaggaggaggctgagcgtctggagcgagagcgccgtctggcagaagaggccaaatcggctctgctgcagcagtcggagagccagatgaagaaccaggagcatctggcaactgaactggcagagttgacctctaagatctccctgcttgaagatgccaaaaagaagaaagaggatgaagcattagagtggcagacaaaggccactatggtgcaagaggacctggagaagactaaagaggagctgaagaacaaagtcatgtcatcccatgtcacagagccagtgcatggagaaaatgacaacgatgagggtgacgagagcagcgcagaagcctctgcagaactgacctccgcagccgcctataaggacaggagcgaggaggagcgcatgacggaagccgagaagaacgaacgagtgcagaagcatctgctggctcttacttctgagcttgctaatgctcgtgatgagaccaagaagacccagaatgacatcattcatgcagagaatgtacgagcaggacgtgacaaatacaagacgcttcgccagatccgctccggaaacaccaaacagcgaatcgacgagtttgagtgcatgtaa

SEQ ID NO: 20 - DNA, 2631 bp, danio rerio
agttgttcgtcttctttgtgggaccaccagtacttctctgagcgacaaagacgcggcgaaaagtgaaactttgtctccggggtctggaagagatttgaggagaagcttttccacacacgagcggcgtcgcttgttgactgttgcgcgtggacatcagccgatgggaaaaagtttggctttctgttgacttcattgtatcagcttcagtttgtgacacattcgtgaaggaagagaccagataccgacaaaatgccgaaaacgatcagtgttcgtgtgactacgatggatgccgagctggagtttgccatccaacccagcaccacagggaaacagttatttgaccaggtggtgaaaaccattgggctgagggaggtgtggttttttggtcttcagtatcaggacaccaaaggtttttccacctggcttaaactcaacaagaaggttacggctcaagacgtccgcaaggagagtcctttgctgtttaaattcagagcaaagttttacccagaagacgtttctgaggaactgattcaggaggccactcaaagactcttcttccttcaggttaaagagggcatcctgaatgacgatatctactgccctcctgagactgcggttttgctggcctcttatgctgtgcaggccaaatatgcagactacaacaaagatgcccacactcctggatacttgtccaatgagaagctgctcccacagagagttctggagcagcacaagctgaataaggagcaatgggaggaaagaattcaggtctggcacgaggagcacaagggcatgttgagagaggactcaatgatggagtatctgaagatagctcaggatctggagatgtatggtgtgaattacttcagtattaagaataagaagggatcagagctgtggcttggtgtggatgctctgggactcaacatctacgagcagaatgacaagatgacacctaaaattggtttcccctggagtgagatcaggaacatttctttcaacgataagaaatttgtcatcaaacccattgataaaaaagccccggactttgtgttctatgcccagcgtttacgcattaataagaggattctggctctgtgcatggggaaccatgagctttatatgagacgccgtaaaccagacaccatcgaggtccagcagatgaaggcacaggccaaggaggaaaagaaccacaagaagatggaaagggcattgttggaggatgaacgtaaaaagagagagcaggccgaaaaggagaaagagaagattgaaaaagaaaaagaggagctgatggagagactcagagtaatcgaggagcagacaagaaaagctcagcaagagctggaggagcagacccgcaaggcgctggagttagagcaggagcgtaaacgtgctcaggaggaggctgagcgtctggagcgagagcgccgtctggcagaagaggccaaatcggctctgctgcagcagtcggagagccagatgaagaaccaggagcatctggcaactgaactggcagagttgacctctaagatctccctgcttgaagatgccaaaaagaagaaagaggatgaagcattagagtggcagacaaaggccactatggtgcaagaggacctggagaagactaaagaggagctgaagaacaaagtcatgtcatcccatgtcacagagccagtgcatggagaaaatgacaacgatgagggtgacgagagcagcgcagaagcctctgcagaactgacctccgcagccgcctataaggacaggagcgaggaggagcgcatgacggaagccgagaagaacgaacgagtgcagaagcatctgctggctcttacttctgagcttgctaatgctcgtgatgagaccaagaagacccagaatgacatcattcatgcagagaatgtacgagcaggacgtgacaaatacaagacgcttcgccagatccgctccggaaacaccaaacagcgaatcgacgagtttgagtgcatgtaatgtaccagcgtcagccagcagggggagctacagcctgctttttgcatcatgcctgcagaggaactacagtacccacaaactacagaaccccttcaacagaaaataaagccaaaaatagaaataatgctgcacaattcaaaaatccctttaattgatgagtgttgtttgggttacttcattatgataagggagttttttttcttttgttggatttgtgttttgatctaggtgttacatttttttttaatatggaaaacattaacggtttactgatttaaccgaattaaagggttttaacttcagtaaaactgggaatcctgtaggaattgaagtaaaagaataaaacattccagtattaagatagaaagcctatggagtcaatctagtgccataaatacttgcaaattaaaagtttgcaaacaaaaaattaagtattgagctaaaacaaaaatttaataaatgcaaatctgcaaatccccaaaaatcatgaagcaaaaaattatttttgaaaaaaaaaaattacataaccaaataaagaactgcaaagggaaaattaagttttgaaaaataaaaatgaattttttcaacaaaaacgaactgcaaaaaaaacaaactgtgcaaaaaaaaaaaaaaaaaaaaaaa

SEQ ID NO: 21 - protein, 579 aa, danio rerio
MPKTISVRVTTMDAELEFAIQPSTTGKQLFDQVVKTIGLREVWFFGLQYQDTKGFSTWLKLNKKVTAQDVRKESPLLFKFRAKFYPEDVSEELIQEATQRLFFLQVKEGILNDDIYCPPETAVLLASYAVQAKYADYNKDAHTPGYLSNEKLLPQRVLEQHKLNKEQWEERIQVWHEEHKGMLREDSMMEYLKIAQDLEMYGVNYFSIKNKKGSELWLGVDALGLNIYEQNDKMTPKIGFPWSEIRNISFNDKKFVIKPIDKKAPDFVFYAQRLRINKRILALCMGNHELYMRRRKPDTIEVQQMKAQAKEEKNHKKMERALLEDERKKREQAEKEKEKIEKEKEELMERLRVIEEQTRKAQQELEEQTRKALELEQERKRAQEEAERLERERRLAEEAKSALLQQSESQMKNQEHLATELAELTSKISLLEDAKKKKEDEALEWQTKATMVQEDLEKTKEELKNKVMSSHVTEPVHGENDNDEGDESSAEASAELTSAAAYKDRSEEERMTEAEKNERVQKHLLALTSELANARDETKKTQNDIIHAENVRAGRDKYKTLRQIRSGNTKQRIDEFECM

SEQ ID NO: 22 - DNA, 1701 bp, mus musculus
atggatgcagagctggagtttgccattcagcccaacaccactggcaagcagctgtttgaccaggtggtgaaaactattggtttgagggaagtttggttctttggtctgcagtaccaggacacaaaagctttctctacttggctgaaactcaataagaaggtgactgcacaggatgtgcggaaggaaagtccattgctcttcaagttccgggccaagttctacccagaggatgtatctgaagaactgatccaggatatcacccagcgcctgttctttctgcaagtgaaggagggcattctcaatgacgacatttactgtccacctgaaactgcggtcctgttggcttcttatgccgtccagtctaagtatggtgacttcaataaggaagtgcacaagtctggctacctggctggagataagttgcttccccaaagagtcttggagcagcacaaactcaacaaggaccagtgggaagagaggatccaggtgtggcatgaggagcaccgtggcatgctcagggaggatgctgtcctggaatatctcaagattgctcaagacctggaaatgtatggtgtgaactatttcagcatcaagaacaagaaaggctcagagctatggctgggcgtggatgccttgggtctcaacatctatgagcagaatgacagactgactcctaagattggcttcccgtggagtgaaatcaggaatatctctttcaatgataagaaatttgtcatcaagcccattgacaaaaaggccccggactttgtgttctatgctccccggcttcggattaacaagcggatcttggccctgtgcatgggaaatcatgagctgtacatgcgtcggcgcaagcctgacaccattgaggtgcagcagatgaaggcccaggctcgggaagagaagcaccagaagcagatggagcgtgctctcctggaaaatgagaagaagaagcgtgacgtggctgagaaagagaaggagaagattgagcgggagaaggaagagctgatggagaagctgaagcagattgaggagcagactaagaaggctcagcaagagctggaagagcagacccgcagccccttagaacttgagcaggaacggaagcgtgcccagagtgaggccgaaaagctagccaaggagcgtcaagaagctgaagaagccaaagaggccctgctgcaggcttctcgggaccagaagaagacccaggaacagctggcttcagaaatggcagagctgacggcacggatctcccagttggaaatggctcgaaagaagaaggaaagtgaggctgtggaatggcagcaaaaggcccagatggtacaggaagacttggagaagactcgtgctgagctgaagactgccatgagtacacctcatgtggcagagcctgctgagaatgaacatgatgagcaggatgagaatggagcagaggccagtgccgagctgcgggctgatgctatggccaaggaccgcagtgaggaggaacgtaccactgaggcagagaagaatgagcgtgtgcagaagcatctgaaggcccttacttcagagctggccaatgcccgagatgagtccaagaagactgccaatgacatgatccatgctgagaacatgcgactgggacgagacaaatacaagaccctgcgccagatccggcagggcaacaccaaacaacgcattgatgagtttgagtccatgtag

SEQ ID NO: 23 - DNA, 2365 bp, mus musculus
atggatgcagagctggagtttgccattcagcccaacaccactggcaagcagctgtttgaccaggtggtgaaaactattggtttgagggaagtttggttctttggtctgcagtaccaggacacaaaagctttctctacttggctgaaactcaataagaaggtgactgcacaggatgtgcggaaggaaagtccattgctcttcaagttccgggccaagttctacccagaggatgtatctgaagaactgatccaggatatcacccagcgcctgttctttctgcaagtgaaggagggcattctcaatgacgacatttactgtccacctgaaactgcggtcctgttggcttcttatgccgtccagtctaagtatggtgacttcaataaggaagtgcacaagtctggctacctggctggagataagttgcttccccaaagagtcttggagcagcacaaactcaacaaggaccagtgggaagagaggatccaggtgtggcatgaggagcaccgtggcatgctcagggaggatgctgtcctggaatatctcaagattgctcaagacctggaaatgtatggtgtgaactatttcagcatcaagaacaagaaaggctcagagctatggctgggcgtggatgccttgggtctcaacatctatgagcagaatgacagactgactcctaagattggcttcccgtggagtgaaatcaggaatatctctttcaatgataagaaatttgtcatcaagcccattgacaaaaaggccccggactttgtgttctatgctccccggcttcggattaacaagcggatcttggccctgtgcatgggaaatcatgagctgtacatgcgtcggcgcaagcctgacaccattgaggtgcagcagatgaaggcccaggctcgggaagagaagcaccagaagcagatggagcgtgctctcctggaaaatgagaagaagaagcgtgacgtggctgagaaagagaaggagaagattgagcgggagaaggaagagctgatggagaagctgaagcagattgaggagcagactaagaaggctcagcaagagctggaagagcagacccgcagccccttagaacttgagcaggaacggaagcgtgcccagagtgaggccgaaaagctagccaaggagcgtcaagaagctgaagaagccaaagaggccctgctgcaggcttctcgggaccagaagaagacccaggaacagctggcttcagaaatggcagagctgacggcacggatctcccagttggaaatggctcgaaagaagaaggaaagtgaggctgtggaatggcagcaaaaggcccagatggtacaggaagacttggagaagactcgtgctgagctgaagactgccatgagtacacctcatgtggcagagcctgctgagaatgaacatgatgagcaggatgagaatggagcagaggccagtgccgagctgcgggctgatgctatggccaaggaccgcagtgaggaggaacgtaccactgaggcagagaagaatgagcgtgtgcagaagcatctgaaggcccttacttcagagctggccaatgcccgagatgagtccaagaagactgccaatgacatgatccatgctgagaacatgcgactgggacgagacaaatacaagaccctgcgccagatccggcagggcaacaccaaacaacgcattgatgagtttgagtccatgtagtgggcgtccagccttcaggggcccctcctccttcttccttgttccccacactcccatagttttgcctaactaacactgtgctggagccactaactagaaaagccttggagccatgccaaacgttcagtatagccatgggaccaaacttaatctccccactcataccctcgggcaaacaaatggcccactgtggtgccaatggaatctccttttccttctttgtcacactcattcaacctagctctctagaatagagcatttctcccgcccagctcagagacacgcattccttttggtttgacaagcaccccctccccatatacacttactgttgtcctctgggactcatgtgtgaagtaggttaacagctagctcccatcccttgccagtcctgtgatttgcaaagattggaatatttgtgttgtttagggacagaaggagggaattggctttatcatttagtagtcagatgacctaggatttagcctcttaattaagtctatcccttttacagttacttaggctttgtaccgattggaggataaaagagatgtttagccattcttatttcttctacctcccatattggacctattacaaatctagtcccagtgggccttgtctctgtgttagggatttagttctaatcaagatgggatgagag

SEQ ID NO: 24 - protein, 579 aa, mus musculus; identical to SEQ ID NO: 21

SEQ ID NO: 25 - DNA, 1734 bp, homo sapiens
atgcccaaaacgatcagtgtgcgtgtgaccaccatggatgcagagctggagtttgccatccagcccaacaccaccgggaagcagctatttgaccaggtggtgaaaactattggcttgagggaagtttggttctttggtctgcagtaccaggacactaaaggtttctccacctggctgaaactcaataagaaggtgactgcccaggatgtgcggaaggaaagccccctgctctttaagttccgtgccaagttctaccctgaggatgtgtccgaggaattgattcaggacatcactcagcgcctgttctttctgcaagtgaaagagggcattctcaatgatgatatttactgcccgcctgagaccgctgtgctgctggcctcgtatgctgtccagtctaagtatggcgacttcaataaggaagtgcataagtctggctacctggccggagacaagttgctcccgcagagagtcctggaacagcacaaactcaacaaggaccagtgggaggagcggatccaggtgtggcatgaggaacaccgtggcatgctcagggaggatgctgtcctggaatatctgaagattgctcaagatctggagatgtatggtgtgaactacttcagcatcaagaacaagaaaggctcagagctgtggctgggggtggatgccctgggtctcaacatctatgagcagaatgacagactaactcccaagataggcttcccctggagtgaaatcaggaacatctctttcaatgataagaaatttgtcatcaagcccattgacaaaaaagccccggacttcgtcttctatgctccccggctgcggattaacaagcggatcttggccttgtgcatggggaaccatgaactatacatgcgccgtcgcaagcctgataccattgaggtgcagcagatgaaggcacaggcccgggaggagaagcaccagaagcagatggagcgtgctatgctggaaaatgagaagaagaagcgtgaaatggcagagaaggagaaagagaagattgaacgggagaaggaggagctgatggagaggctgaagcagatcgaggaacagactaagaaggctcagcaagaactggaagaacagacccgtagggctctggaacttgagcaggaacggaagcgtgcccagagcgaggctgaaaagctggccaaggagcgtcaagaagctgaagaggccaaggaggccttgctgcaggcctcccgggaccagaaaaagactcaggaacagctggccttggaaatggcagagctgacagctcgaatctcccagctggagatggcccgacagaagaaggagagtgaggctgtggagtggcagcagaaggcccagatggtacaggaagacttggagaagacccgtgctgagctgaagactgccatgagtacacctcatgtggcagagcctgctgagaatgagcaggatgagcaggatgagaatggggcagaggctagtgctgacctacgggctgatgctatggccaaggaccgcagtgaggaggaacgtaccactgaggcagagaagaatgagcgtgtgcagaagcacctgaaggccctcacttcggagctggccaatgccagagatgagtccaagaagactgccaatgacatgatccatgctgagaacatgcgactgggccgagacaaatacaagaccctgcgccagatccggcagggcaacaccaagcagcgcattgacgaatttgagtctatgtaa

SEQ ID NO: 26 - DNA, 3879 bp, homo sapiens
ggcacgaggccagccgaatccaagccgtgtgtactgcgtgctcagcactgcccgacagtcctagctaaacttcgccaactccgctgcctttgccgccaccatgcccaaaacgatcagtgtgcgtgtgaccaccatggatgcagagctggagtttgccatccagcccaacaccaccgggaagcagctatttgaccaggtggtgaaaactattggcttgagggaagtttggttctttggtctgcagtaccaggacactaaaggtttctccacctggctgaaactcaataagaaggtgactgcccaggatgtgcggaaggaaagccccctgctctttaagttccgtgccaagttctaccctgaggatgtgtccgaggaattgattcaggacatcactcagcgcctgttctttctgcaagtgaaagagggcattctcaatgatgatatttactgcccgcctgagaccgctgtgctgctggcctcgtatgctgtccagtctaagtatggcgacttcaataaggaagtgcataagtctggctacctggccggagacaagttgctcccgcagagagtcctggaacagcacaaactcaacaaggaccagtgggaggagcggatccaggtgtggcatgaggaacaccgtggcatgctcagggaggatgctgtcctggaatatctgaagattgctcaagatctggagatgtatggtgtgaactacttcagcatcaagaacaagaaaggctcagagctgtggctgggggtggatgccctgggtctcaacatctatgagcagaatgacagactaactcccaagataggcttcccctggagtgaaatcaggaacatctctttcaatgataagaaatttgtcatcaagcccattgacaaaaaagccccggacttcgtcttctatgctccccggctgcggattaacaagcggatcttggccttgtgcatggggaaccatgaactatacatgcgccgtcgcaagcctgataccattgaggtgcagcagatgaaggcacaggcccgggaggagaagcaccagaagcagatggagcgtgctatgctggaaaatgagaagaagaagcgtgaaatggcagagaaggagaaagagaagattgaacgggagaaggaggagctgatggagaggctgaagcagatcgaggaacagactaagaaggctcagcaagaactggaagaacagacccgtagggctctggaacttgagcaggaacggaagcgtgcccagagcgaggctgaaaagctggccaaggagcgtcaagaagctgaagaggccaaggaggccttgctgcaggcctcccgggaccagaaaaagactcaggaacagctggccttggaaatggcagagctgacagctcgaatctcccagctggagatggcccgacagaagaaggagagtgaggctgtggagtggcagcagaaggcccagatggtacaggaagacttggagaagacccgtgctgagctgaagactgccatgagtacacctcatgtggcagagcctgctgagaatgagcaggatgagcaggatgagaatggggcagaggctagtgctgacctacgggctgatgctatggccaaggaccgcagtgaggaggaacgtaccactgaggcagagaagaatgagcgtgtgcagaagcacctgaaggccctcacttcggagctggccaatgccagagatgagtccaagaagactgccaatgacatgatccatgctgagaacatgcgactgggccgagacaaatacaagaccctgcgccagatccggcagggcaacaccaagcagcgcattgacgaatttgagtctatgtaatgggcacccagcctctagggacccctcctccctttttccttgtccccacactcctacacctaactcacctaactcatactgtgctggagccactaactagagcagccctggagtcatgccaagcatttaatgtagccatgggaccaaacctagccccttagcccccacccacttccctgggcaaatgaatggctcactatggtgccaatggaacctcctttctcttctctgttccattgaatctgtatggctagaatatcctacttctccagcctagaggtactttccacttgattttgcaaatgcccttacacttactgttgtcctatgggagtcaagtgtggagtaggttggaagctagctcccctcctctcccctccactgtcttcttcaggtcctgagattacacggtggagtgtatgcggtctaggaatgagacaggacctagatatcttctccagggatgtcaactgacctaaaatttgccctcccatcccgtttagagttatttaggctttgtaacgattgggggaataaaaagatgttcagtcatttttgtttctacctcccagatcggatctgttgcaaactcagcctcaataagccttgtcgttgactttagggactcaatttctccccagggtggatgggggaaatggtgccttcaagaccttcaccaaacatactagaagggcattggccattctattgtggcaaggctgagtagaagatcctaccccaattccttgtaggagtataggccggtctaaagtgagctctatgggcagatctaccccttacttattattccagatctgcagtcacttcgtgggatctgcccctccctgcttcaatacccaaatcctctccagctataacagtagggatgagtacccaaaagctcagccagccccatcaggactcttgtgaaaagagaggatatgttcacacctagcgtcagtattttccctgctaggggttttaggtctcttcccctctcagagctacttgggccatagctcctgctccacagccatcccagccttggcatctagagcttgatgccagtaggctcaactagggagtgagtgcaaaaagctgagtatggtgagagaagcctgtgccctgatccaagtttactcaaccctctcaggtgaccaaaatccccttctcatcactcccctcaaagaggtgactgggccctgcctctgtttgacaaacctctaacccaggtcttgacaccagctgttctgtcccttggagctgtaaaccagagagctgctgggggattctggcctagtcccttccacacccccaccccttgctctcaacccaggagcatccacctccttctctgtctcatgtgtgctcttcttctttctacagtattatgtactctactgatatctaaatattgatttctgccttccttgctaatgcaccattagaagatattagtcttggggcaggatgattttggcctcattactttaccacccccacacctggaaagcatatactatattacaaaatgacattttgccaaaattattaatataagaagctttcagtattagtgatgtcatctgtcactataggtcatacaatccattcttaaagtacttgttatttgtttttattattactgtttgtcttctccccagggttcagtccctcaaggggccatcctgtcccaccatgcagtgccccctagcttagagcctccctcaattccccctggccaccaccccccactctgtgcctgaccttgaggagtcttgtgtgcattgctgtgaattagctcacttggtgatatgtcctatattggctaaattgaaacctggaattgtggggcaatctattaatagctgccttaaagtcagtaacttacccttagggaggctgggggaaaaggttagattttgtattcaggggttttttgtgtactttttgggtttttaaaaaattgtttttggaggggtttatgctcaatccatgttctatttcagtgccaataaaatttaggtgacttcaaaaaaaaaaaa

SEQ ID NO: 27 - protein, 577 aa, homo sapiens
MPKTISVRVTTMDAELEFAIQPNTTGKQLFDQVVKTIGLREVWFFGLQYQDTKGFSTWLKLNKKVTAQDVRKESPLLFKFRAKFYPEDVSEELIQDITQRLFFLQVKEGILNDDIYCPPETAVLLASYAVQSKYGDFNKEVHKSGYLAGDKLLPQRVLEQHKLNKDQWEERIQVWHEEHRGMLREDAVLEYLKIAQDLEMYGVNYFSIKNKKGSELWLGVDALGLNIYEQNDRLTPKIGFPWSEIRNISFNDKKFVIKPIDKKAPDFVFYAPRLRINKRILALCMGNHELYMRRRKPDTIEVQQMKAQAREEKHQKQMERAMLENEKKKREMAEKEKEKIEREKEELMERLKQIEEQTKKAQQELEEQTRRALELEQERKRAQSEAEKLAKERQEAEEAKEALLQASRDQKKTQEQLALEMAELTARISQLEMARQKKESEAVEWQQKAQMVQEDLEKTRAELKTAMSTPHVAEPAENEQDEQDENGAEASADLRADAMAKDRSEEERTTEAEKNERVQKHLKALTSELANARDESKKTANDMIHAENMRLGRDKYKTLRQIRQGNTKQRIDEFESM

SEQ ID NO: 28 - DNA, 1335 bp, danio rerio
atgagggtctttagcaaatcgctattgctgattgctgttggcattttaatgtcccagcagacactggctcactcccaccatcatcatggacatggtgatggtggatgccatggtcactcacatggtggagcgaagatgcatcacggggcaagcaaatggagtgctgaagctaatctgcctcatgctgaagaggagcatcacgtacacgatcacgggcacacacataatcatgcacacgatcacgggcatgcacacagtcatggagacattcatgatcatggacatgcacacaagcatggacacgcacacgatcatggagctgagaaatcgaagaaagttgtggaagcaggcaaacggaacatggtggagctctggatgcaggccattggagccaccctgctcattagtgctgcaccatttctgatactcttcctcattccagtacagtcaaacactgaccagcaccagaacctgcttaaagtgctcttgagttttgcatctggtggtttgcttggagatgcattccttcatctcattcctcatgcactggaacctcattctcatcacagtcagccacacagcgaggagtcccatggccagtcgcatggtgaggagtcacatggtcactctcatggagctgcccatggtcacatgatgtcagttggtctgtgggtgctaggtggcattgttgcatttctagtggttgagaaatttgttcgtcttttgaaaggaggacattcacactcgcattcccactctccctctgctccaaagtcaaaggatagtgatgaagaagatgacaagaaaggacaaaagaagggagagaaagacaaagttgtctctcaacagaaacctacaaagaaaacagtagagacaagttctgatattaaagtgtctggttatctgaacctggctgctgatttcactcacaatttcacggatggtcttgcaattggagcatctttcctggttggtccagctgttggtgctgttaccaccatcaccatcctcttacatgaagtaccacatgaaattggagactttgcaattcttgtccaatcaggctgcaccaagaggaaggctatgtgtcttcagcttctcactgctgttggtgcattagctggaactgcctgctccttattggctgaaggagtgggtgacgcggccacagcgtggatcctcccctttactgccggaggctttgtttacattgcgactgtcacagttctcccagagctgctggctggacactctagtttctggcagtcactcctggagatcctggctcttctgtttggagtgggaatgatggtgctgattgcagagtatgagtga

SEQ ID NO: 29 - DNA, 1994 bp, danio rerio
aaatgtgttgactgaatgccacttcgctttgagtgtgaccagcatcttaactgctttcactctttaccagaataagctaatttatattagttttcgcagcttataccgttgctagacgtggatgcggttagcagattgctaacttattcagtgcacggtactggcgcgtgctttggctttttcggcatcactagcaggaacaatgagggtctttagcaaatcgctattgctgattgctgttggcattttaatgtcccagcagacactggctcactcccaccatcatcatggacatggtgatggtggatgccatggtcactcacatggtggagcgaagatgcatcacggggcaagcaaatggagtgctgaagctaatctgcctcatgctgaagaggagcatcacgtacacgatcacgggcacacacataatcatgcacacgatcacgggcatgcacacagtcatggagacattcatgatcatggacatgcacacaagcatggacacgcacacgatcatggagctgagaaatcgaagaaagttgtggaagcaggcaaacggaacatggtggagctctggatgcaggccattggagccaccctgctcattagtgctgcaccatttctgatactcttcctcattccagtacagtcaaacactgaccagcaccagaacctgcttaaagtgctcttgagttttgcatctggtggtttgcttggagatgcattccttcatctcattcctcatgcactggaacctcattctcatcacagtcagccacacagcgaggagtcccatggccagtcgcatggtgaggagtcacatggtcactctcatggagctgcccatggtcacatgatgtcagttggtctgtgggtgctaggtggcattgttgcatttctagtggttgagaaatttgttcgtcttttgaaaggaggacattcacactcgcattcccactctccctctgctccaaagtcaaaggatagtgatgaagaagatgacaagaaaggacaaaagaagggagagaaagacaaagttgtctctcaacagaaacctacaaagaaaacagtagagacaagttctgatattaaagtgtctggttatctgaacctggctgctgatttcactcacaatttcacggatggtcttgcaattggagcatctttcctggttggtccagctgttggtgctgttaccaccatcaccatcctcttacatgaagtaccacatgaaattggagactttgcaattcttgtccaatcaggctgcaccaagaggaaggctatgtgtcttcagcttctcactgctgttggtgcattagctggaactgcctgctccttattggctgaaggagtgggtgacgcggccacagcgtggatcctcccctttactgccggaggctttgtttacattgcgactgtcacagttctcccagagctgctggctggacactctagtttctggcagtcactcctggagatcctggctcttctgtttggagtgggaatgatggtgctgattgcagagtatgagtgagcgaggaagccatgtttaaaagaagaacttggaatatgaggcgcagtgaccaatgtatggtcaccggaagcgcagagaaactttgaaatgtatgttagcattgaccacaggaggaaaacctgcgtaagaaataaaaaaagtgcttttatgagaaaactactcatttatgcaaagacctgcatgaatggatcacatgaaacaacctcaccagggaccaaaatttagatctaatacatgcatacaaatcataggtatacactaaagagactgatagaatcagaatgtgcggaccattacgttccaaagttactatgtctccagtcactaaaacatgttttgctaatcaatttatttgcttatcaagaaattgcagtaatgttttgatttgaaaaatcatttgactgcacttcacgcaataaagaagtatgtgattctttctaaaaaaaaaaaaaaaaaa

SEQ ID NO: 30 - protein, 444 aa, danio rerio
MRVFSKSLLLIAVGILMSQQTLAHSHHHHGHGDGGCHGHSHGGAKMHHGASKWSAEANLPHAEEEHHVHDHGHTHNHAHDHGHAHSHGDIHDHGHAHKHGHAHDHGAEKSKKVVEAGKRNMVELWMQAIGATLLISAAPFLILFLIPVQSNTDQHQNLLKVLLSFASGGLLGDAFLHLIPHALEPHSHHSQPHSEESHGQSHGEESHGHSHGAAHGHMMSVGLWVLGGIVAFLVVEKFVRLLKGGHSHSHSHSPSAPKSKDSDEEDDKKGQKKGEKDKVVSQQKPTKKTVETSSDIKVSGYLNLAADFTHNFTDGLAIGASFLVGPAVGAVTTITILLHEVPHEIGDFAILVQSGCTKRKAMCLQLLTAVGALAGTACSLLAEGVGDAATAWILPFTAGGFVYIATVTVLPELLAGHSSFWQSLLEILALLFGVGMMVLIAEYE

SEQ ID NO: 31 - DNA, 1431 bp, mus musculus
atgaccatgggcctgcgggccccccactgggtggctgtgggactgctgacctgggcggctttggggctgctggtggccggacacgagggtcatggtgacctgcacaaagatgtggaagaggacttccatggccacagtcacgggcactcacatgaagatttccaccatggccacagccacgggcactcgcatgaagatttccaccatggccacggccacactcatgaaagcatctggcatgggcatgcccacagccacgaccatggacactcacgtgaggaattacaccatggccatagccatggccactcccacgatagcctccaccacggaggacatggacatgcccatcgtgaacatagccatgggacttctagggaggctggggctccaggcatcaaacaccacctggacactgtcaccctctgggcctacgcactgggggccacagtgctgatctccgcagctccgttcttcgtgctgttcctcatcccagtagaatctaactctcccaggcaccgctctctgctccagatcctgctcagttttgcttccggggggctcctgggtgatgcgttcctccacctcatcccgcatgccttggaacctcattctcaccacgctccagagcagcctggacatggacactcccacagtggccagggccccattctctctgtggggctgtgggttctcagtgggattgtcgccttcctcgtggtggagaaatttgtgagacacgtgaaaggaggacatggacacagtcacggacacggagacaggcatgcgcatggagacagtcacacccatggagatagacatgagtgttcttcaaaggaaaagcccagcacagaggaagagaaggaagtgggcgggttgcggaaaaggagaggaggaaacactgggcccagagatggcccggtgaaacctcagagccctgaagaagaaaaagcaggctcagacctgcgtgtgtctgggtacctgaatctggctgctgacttggcacacaacttcacagacggtctggccattggtgcttcctttcgtgggggccgagggctagggatcctgaccacaatgacagtcctgctgcacgaagtgcctcatgaggttggggattttgccatcctggtccagtctggctgcagcaagaagcaggcgatgcgtctgcaactcgtgactgcaattggagcattggcaggcactgcctgtgcccttctcaccgagggaggggcagtggacagtgacgtggcaggtggtgcaggtcctggctgggtcctgccattcactgcaggcggatttatctacgtagcaacagtgtctgtgctgcctgagctattgagagaggcatctccactgcagtcactgttggaggtgctggggctgctggggggtgttgccatgatggtactgattgcccatcttgagtga

SEQ ID NO: 32 - DNA, 2254 bp, mus musculus
tcccggagccggtgagaggtccctgctgctcccttacggcgctttccaggcctttaccccatccagtgggccatagaggcgcgggcccagagagaccgtaaagttgctgatcaaaggctagagcggtgtcgggggtggggggctgcatccaggaagggtgttggggatgaggtggaccggccttggggacaatgtaagagcggagcaagtagtatagaggaagggcttcaagggacgcggatcccgaataggtagattgagagtcaagtcgagtcgtctcttgttcctccggtcagcgtgatgaccatgggcctgcgggccccccactgggtggctgtgggactgctgacctgggcggctttggggctgctggtggccggacacgagggtcatggtgacctgcacaaagatgtggaagaggacttccatggccacagtcacgggcactcacatgaagatttccaccatggccacagccacgggcactcgcatgaagatttccaccatggccacggccacactcatgaaagcatctggcatgggcatgcccacagccacgaccatggacactcacgtgaggaattacaccatggccatagccatggccactcccacgatagcctccaccacggaggacatggacatgcccatcgtgaacatagccatgggacttctagggaggctggggctccaggcatcaaacaccacctggacactgtcaccctctgggcctacgcactgggggccacagtgctgatctccgcagctccgttcttcgtgctgttcctcatcccagtagaatctaactctcccaggcaccgctctctgctccagatcctgctcagttttgcttccggggggctcctgggtgatgcgttcctccacctcatcccgcatgccttggaacctcattctcaccacgctccagagcagcctggacatggacactcccacagtggccagggccccattctctctgtggggctgtgggttctcagtgggattgtcgccttcctcgtggtggagaaatttgtgagacacgtgaaaggaggacatggacacagtcacggacacggagacaggcatgcgcatggagacagtcacacccatggagatagacatgagtgttcttcaaaggaaaagcccagcacagaggaagagaaggaagtgggcgggttgcggaaaaggagaggaggaaacactgggcccagagatggcccggtgaaacctcagagccctgaagaagaaaaagcaggctcagacctgcgtgtgtctgggtacctgaatctggctgctgacttggcacacaacttcacagacggtctggccattggtgcttcctttcgtgggggccgagggctagggatcctgaccacaatgacagtcctgctgcacgaagtgcctcatgaggttggggattttgccatcctggtccagtctggctgcagcaagaagcaggcgatgcgtctgcaactcgtgactgcaattggagcattggcaggcactgcctgtgcccttctcaccgagggaggggcagtggacagtgacgtggcaggtggtgcaggtcctggctgggtcctgccattcactgcaggcggatttatctacgtagcaacagtgtctgtgctgcctgagctattgagagaggcatctccactgcagtcactgttggaggtgctggggctgctggggggtgttgccatgatggtactgattgcccatcttgagtgaggggtgaggtgacctgtccttcccctaactcctaataaaggcagttggtagtcctggccagtgctaatgccagaaggagtgtcagccttggagattagcagagcctctgtttgaggccttagaggtatgaggatcatacggagcatgagaggccagaaggaccacggagtgggagactgcccagcagcgttgttgcttttggaaaaatcaatgggaccatgaagaagactgggaagacggtagcctacccgatggtccctaatctacgatttctcagcgccagcgtgccgcaggggtccctccaaggcccatctccctctgaactagttagtgatggcttcagggaagacctggcagaaggactggggtagacatcaatcgtgtgtcctgatttgaagggggaggggggctccttgggaagatgtctcagcctgattttttgtctctactcattttataccactgtttgaatgggtgcggaggaacggtgaccaggaataaaagaccttggatcttcggcccc

SEQ ID NO: 33 - protein, 476 aa, mus musculs
MTMGLRAPHWVAVGLLTWAALGLLVAGHEGHGDLHKDVEEDFHGHSHGHSHEDFHHGHSHGHSHEDFHHGHGHTHESIWHGHAHSHDHGHSREELHHGHSHGHSHDSLHHGGHGHAHREHSHGTSREAGAPGIKHHLDTVTLWAYALGATVLISAAPFFVLFLIPVESNSPRHRSLLQILLSFASGGLLGDAFLHLIPHALEPHSHHAPEQPGHGHSHSGQGPILSVGLWVLSGIVAFLVVEKFVRHVKGGHGHSHGHGDRHAHGDSHTHGDRHECSSKEKPSTEEEKEVGGLRKRRGGNTGPRDGPVKPQSPEEEKAGSDLRVSGYLNLAADLAHNFTDGLAIGASFRGGRGLGILTTMTVLLHEVPHEVGDFAILVQSGCSKKQAMRLQLVTAIGALAGTACALLTEGGAVDSDVAGGAGPGWVLPFTAGGFIYVATVSVLPELLREASPLQSLLEVLGLLGGVAMMVLIAHLE

SEQ ID NO: 34 - DNA, 1410 bp, homo sapiens
atggccagaggcctgggggccccccactgggtggccgtgggactgctgacctgggcgaccttggggcttctggtggctggactcgggggtcatgacgacctgcacgacgatctgcaagaggacttccatggccacagccacaggcactcacatgaagatttccaccatggccacagccatgcccatggccatggccacactcacgagagcatctggcatggacatacccacgatcacgaccatggacattcacatgaggatttacaccatggccatagccatggctactcccatgagagcctctaccacagaggacatggacatgaccatgagcatagccatggaggctatggggagtctggggctccaggcatcaagcaggacctggatgctgtcactctctgggcttatgcactgggggccacagtgctgatctcagcagctccattttttgtcctcttccttatccccgtggagtcgaactctccccggcatcgctctctacttcagatcttgctcagttttgcttccggtgggctcctgggagatgctttcctgcacctcattcctcatgctcttgaacctcattctcaccacactctggagcaacccggacatggacactcccacagtggccagggccccattctgtctgtgggactgtgggttctcagtggaattgttgcctttcttgtcgtggagaaatttgtgagacatgtgaaaggaggacatggtcacagtcatggacatggacacgctcacagtcatacacgtggaagtcatggacatggaagacaagagcgttctaccaaggagaagcagagctcagaggaagaagaaaaggaaacaagaggggttcagaagaggcgaggagggagcacagtacccaaagatgggccagtgagacctcagaacgctgaagaagaaaaaagaggcttagacctgcgtgtgtcggggtacctgaatctggctgctgacttggcacacaacttcactgatggtctggccattggggcttcctttcgagggggccggggactagggatcctgaccacaatgactgtcctgctacatgaagtgccccacgaggtcggagactttgccatcttggtccagtctggctgcagcaaaaagcaggcgatgcgtctgcaactactgacagcagtaggggcactggcaggcacagcctgtgcccttctcactgaaggaggagcagtgggcagtgaaattgcaggtggtgcaggtcctggctgggtcctgccatttactgcaggtggctttatctacgtagcaacagtgtctgtgttgcccgagctgctgagggaggcatcaccattgcaatcacttctggaggtgctggggctgctggggggagttatcatgatggtgctgattgcccaccttgagtga

SEQ ID NO: 35 - DNA, 2332 bp, homo sapiens
tctctgttttttctctaccatcctttccaggccttttcctcacctaatgagtcgtagagacgagggcccagagagtctgtaaagtggctggtgaaagattagtgtcccagggccctacatccgggaggtggttcgggataaagagaactagtcttgggaacaatgtaggtgggaacttaagggaatgggagagcggcccatagaggtggacggagggcgcgattggagtaaagcggaccctgtgtaggtatagagttgagtcaagtggagtcactgcctctgtccctctggtcagcgtgatggccagaggcctgggggccccccactgggtggccgtgggactgctgacctgggcgaccttggggcttctggtggctggactcgggggtcatgacgacctgcacgacgatctgcaagaggacttccatggccacagccacaggcactcacatgaagatttccaccatggccacagccatgcccatggccatggccacactcacgagagcatctggcatggacatacccacgatcacgaccatggacattcacatgaggatttacaccatggccatagccatggctactcccatgagagcctctaccacagaggacatggacatgaccatgagcatagccatggaggctatggggagtctggggctccaggcatcaagcaggacctggatgctgtcactctctgggcttatgcactgggggccacagtgctgatctcagcagctccattttttgtcctcttccttatccccgtggagtcgaactctccccggcatcgctctctacttcagatcttgctcagttttgcttccggtgggctcctgggagatgctttcctgcacctcattcctcatgctcttgaacctcattctcaccacactctggagcaacccggacatggacactcccacagtggccagggccccattctgtctgtgggactgtgggttctcagtggaattgttgcctttcttgtcgtggagaaatttgtgagacatgtgaaaggaggacatggtcacagtcatggacatggacacgctcacagtcatacacgtggaagtcatggacatggaagacaagagcgttctaccaaggagaagcagagctcagaggaagaagaaaaggaaacaagaggggttcagaagaggcgaggagggagcacagtacccaaagatgggccagtgagacctcagaacgctgaagaagaaaaaagaggcttagacctgcgtgtgtcggggtacctgaatctggctgctgacttggcacacaacttcactgatggtctggccattggggcttcctttcgagggggccggggactagggatcctgaccacaatgactgtcctgctacatgaagtgccccacgaggtcggagactttgccatcttggtccagtctggctgcagcaaaaagcaggcgatgcgtctgcaactactgacagcagtaggggcactggcaggcacagcctgtgcccttctcactgaaggaggagcagtgggcagtgaaattgcaggtggtgcaggtcctggctgggtcctgccatttactgcaggtggctttatctacgtagcaacagtgtctgtgttgcccgagctgctgagggaggcatcaccattgcaatcacttctggaggtgctggggctgctggggggagttatcatgatggtgctgattgcccaccttgagtgaggggtggataaactacccctgccccaaacctctacccctaactccaggtcaggggtgcgtagaggttgggggccctggccagggacatctgccaaaggaaggaactgtagcctgggagaatggttactttggcattagggccttcaagggctggcagtcttacagaggctggagcggtgagaatgagaggccagagggaccatagtgttgggcactgtctgaccatgttgcatttggaaggctaaatggggccatgaagaaggctggaagggacagggggtgatggcagcctacctggtgtcccctaccccacctgttctcggagaaccaagttgctacacaggaagttctccaaggtccagtttcctttctcccaccagttggtggaggcttcagggaagaccagagtcctggacagagagggtaacaggaggagtcggggataaacatcaaacatcaatcgtgtgtcctgatttgggagtgattggggggatggggtgggagagggttagttggtattctcatggcctgattttttttgtttctattccttttatatcactgtgtttgaatcgagggggaggggtggtaaccggaaataaagacctccgatcttccgcccc

SEQ ID NO: 36 - protein, 469 aa, homo sapiens
MARGLGAPHWVAVGLLTWATLGLLVAGLGGHDDLHDDLQEDFHGHSHRHSHEDFHHGHSHAHGHGHTHESIWHGHTHDHDHGHSHEDLHHGHSHGYSHESLYHRGHGHDHEHSHGGYGESGAPGIKQDLDAVTLWAYALGATVLISAAPFFVLFLIPVESNSPRHRSLLQILLSFASGGLLGDAFLHLIPHALEPHSHHTLEQPGHGHSHSGQGPILSVGLWVLSGIVAFLVVEKFVRHVKGGHGHSHGHGHAHSHTRGSHGHGRQERSTKEKQSSEEEEKETRGVQKRRGGSTVPKDGPVRPQNAEEEKRGLDLRVSGYLNLAADLAHNFTDGLAIGASFRGGRGLGILTTMTVLLHEVPHEVGDFAILVQSGCSKKQAMRLQLLTAVGALAGTACALLTEGGAVGSEIAGGAGPGWVLPFTAGGFIYVATVSVLPELLREASPLQSLLEVLGLLGGVIMMVLIAHLE

SEQ ID NO: 37 - DNA, 47 bp, Artificial Sequence
  PCR Primer
accacttcctacaacaaagctgggttttttttttttttttttttttv

SEQ ID NO: 38 - DNA, 25 bp, Artificial Sequence
  PCR Primer
cattcatggaaccaaaagtgggtgg

SEQ ID NO: 39 - DNA, 48 bp, Artificial Sequence
  PCR Primer
cgacagaacacgctccagcattgaccacttcctacaacaaagctgggt

SEQ ID NO: 40 - DNA, 22 bp, Artificial Sequence
  PCR Primer
cgacagaacacgctccagcatg

SEQ ID NO: 41 - DNA, 26 bp, Artificial Sequence
  PCR Primer
tgcagccctactcagtttcacattgg

SEQ ID NO: 42 - DNA, 25 bp, Artificial Sequence
  PCR Primer
cgctccagcattgaccacttcctac

SEQ ID NO: 43 - DNA, 18 bp, Artificial Sequence
  PCR Primer
gtaaaacgacggccagtg

SEQ ID NO: 44 - DNA, 19 bp, Artificial Sequence
  PCR Primer
ggaaacagctatgaccatg

SEQ ID NO: 45 - DNA, 25 bp, Artificial Sequence
  Antisense
ggttcctcataattcctcagtcttc

SEQ ID NO: 46 - DNA, 22 bp, Artificial Sequence
  Antisense
gctcgtgaaagcggaaaatcgc

SEQ ID NO: 47 - DNA, 25 bp, Artificial Sequence
  Antisense to VEGF
gtatcaaataaacaaccaagttcat

SEQ ID NO: 48 - DNA, 25 bp, Artificial Sequence
  PCR Primer
caccatggctgagcccgagctcctc

SEQ ID NO: 49 - DNA, 28 bp, Artificial Sequence
  PCR Primer
ccaacacctctcagtagtgaacaggcac

SEQ ID NO: 50 - DNA, 26 bp, Artificial Sequence
  PCR Primer
tgcagatcagtctcgcattatgcagg

SEQ ID NO: 51 - DNA, 26 bp, Artificial Sequence
  PCR Primer
gccaccatgccacctagctcaactaa

SEQ ID NO: 52 - DNA, 18 bp, Artificial Sequence
  PCR Primer
gtaaaacgacggccagtg

SEQ ID NO: 53 - DNA, 19 bp, Artificial Sequence
  PCR Primer
ggaaacagctatgaccatg

SEQ ID NO: 54 - DNA, 25 bp, Artificial Sequence
  Antisense
ccctgctctcgtattcaaatgacgg

SEQ ID NO: 55 - DNA, 25 bp, Artificial Sequence
  Antisense
accgataacgactcgaatcaggatg

SEQ ID NO: 56 - DNA, 25 bp, Artificial Sequence
  Antisense to VEGF
gtatcaaataaacaaccaagttcat

SEQ ID NO: 57 - DNA, 28 bp, Artificial Sequence
  PCR Primer
caccatgccgaaaacgatcagtgttcgt

SEQ ID NO: 58 - DNA, 25 bp, Artificial Sequence
  PCR Primer
cctggttcttcatctggctctccga

SEQ ID NO: 59 - DNA, 25 bp, Artificial Sequence
  PCR Primer
tctactgccctcctgagactgcggt

SEQ ID NO: 60 - DNA, 25 bp, Artificial Sequence
  PCR Primer
cctggttcttcatctggctctccga

SEQ ID NO: 61 - DNA, 18 bp, Artificial Sequence
  PCR Primer
gtaaaacgacggccagtg

SEQ ID NO: 62 - DNA, 19 bp, Artificial Sequence
  PCR Primer
ggaaacagctatgaccatg

SEQ ID NO: 63 - DNA, 25 bp, Artificial Sequence
  Antisense
cggcattttgtcggtatctggtctc

SEQ ID NO: 64 - DNA, 25 bp, Artificial Sequence
  Antisense
acgaatgtgtcacaaactgaagctg

SEQ ID NO: 65 - DNA, 30 bp, Artificial Sequence
  PCR Primer
caccatgagggtctttagcaaatcgctatt

SEQ ID NO: 66 - DNA, 25 bp, Artificial Sequence
  PCR Primer
tcatactctgcaatcagcaccatca

SEQ ID NO: 67 - DNA, 26 bp, Artificial Sequence
  PCR Primer
attcttgtccaatcaggctgcaccaa

SEQ ID NO: 68 - DNA, 26 bp, Artificial Sequence
  PCR Primer
ggtcactgcgcctcatattccaagtt

SEQ ID NO: 69 - DNA, 18 bp, Artificial Sequence
  PCR Primer
gtaaaacgacggccagtg

SEQ ID NO: 70 - DNA, 19 bp, Artificial Sequence
  PCR Primer
ggaaacagctatgaccatg

SEQ ID NO: 71 - DNA, 25 bp, Artificial Sequence
  Antisense
agcgatttgctaaagaccctcattg

SEQ ID NO: 72 - DNA, 25 bp, Artificial Sequence
  Antisense
gcaatctgctaaccgcatccacgtc

(19) United States
(12) Patent Application Publication
(10) Pub. No.: US 2003/0220249 A1
(43) Pub. Date: Nov. 27, 2003

(54) Factors for angiogenesis, vasculogenesis, cartilage formation, bone formation, and methods of use thereof

(76) Inventors: Perry B. Hackett, Shoreview, MN (US); Aidas Nasevicius, Roseville, MN (US); Shannon Wadman, Minneapolis, MN (US); Jeffrey Essner, Minneapolis, MN (US); Jon Larson, Minneapolis, MN (US); Karl J. Clark, Ramsey, MN (US); Sharon Roberg-Perez, Minneapolis, MN (US); Stephen C. Ekker, St. Paul, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/360,849

(22) Filed: Feb. 7, 2003

Related U.S. Application Data
(60) Provisional application No. 60/354,978, filed on Feb. 7, 2002.

(51) Int. Cl. A61K 48/00; A61K 38/18; C12Q 1/68; A01K 67/027; C07H 21/04; C12P 21/02; C12N 5/06; C07K 14/475

(52) U.S. Cl. 514/12; 514/44; 514/81; 514/90; 435/6; 435/69.1; 435/320.1; 435/325; 530/350; 536/23.2; 544/81; 544/82; 800/18; 800/20

(57) ABSTRACT

The application is related to the field of nucleic acids with identified utility, and more particularly, to genes, related nucleic acids, their complements, polypeptides, and methods of using the same for blood vessel, cartilage, and bone formation, as well as inhibition thereof. The application describes discoveries made using the zebrafish embryo technique, as well as other techniques that are described herein. The discoveries include genes, related nucleic acids, and their complements, as well as sequences, polypeptides, other molecules, and methods for using them, e.g., TDE1, PTV, MOESIN, and HKE4. Also described are polypeptide products, inhibition of expression, administration of materials and products, screening procedures, and techniques for making drugs. Moreover, uses of these discoveries in appropriate contexts are set forth.

### RELATED APPLICATIONS

This application claims priority to U.S. Patent No. 60/364,978 entitled “Technology For Identification Of Genes For Angiogenesis And Genes Therefor”, filed, Feb. 07, 2002, hereby incorporated by reference herein.

### FIELD OF USE

This application is related to the field of nucleic acids with identified utility, and more particularly, to genes, related nucleic acids, their complements, polypeptides, and methods of using the same for blood vessel, cartilage, and bone formation, as well as inhibition thereof.

### BACKGROUND

There is a continuing need for new genes and gene products in biotechnical applications, including fields such as medicine, diagnostics, and tools for genomic and proteomic data collection. Although the genome of certain species, most notably mankind, has been sequenced, the raw sequencing data does not identify the genes. More information is required to gainfully use the raw sequencing data.

A recently developed method for discovering new genes and their function involves the use of zebrafish. Zebrafish embryos are injected with antisense molecules that interfere with the function of genes in the embryo. Observations of the embryo are used to discover information about the genes so that they may be used in biotechnical applications. This method allows for many genes to be analyzed and yields insights that are difficult to obtain using other methods. Details of these methods are provided below.

### SUMMARY OF THE INVENTION

This application describes discoveries made using the zebrafish embryo technique, as well as other techniques that are described herein. The discoveries include genes, related nucleic acids, and their complements, as well as sequences, polypeptides, other molecules, and methods for using them. Also described are polypeptide products, inhibition of expression, administration of materials and products, screening procedures, and techniques for making drugs. Moreover, uses of these discoveries in appropriate contexts are set forth.

TABLE 1
INDEX TO CERTAIN GENES SET FORTH HEREIN

| T | | | |
|---|---|---|---|
| SEQ | | | |
| ID NO | Gene | Species | Sequence type |
| 1 | tde1 | zebrafish | Coding Sequence |
| 2 | tde1 | zebrafish | Full Length |
| 3 | TDE1 | zebrafish | Amino Acid |
| 4 | tde1 | Mouse | Coding Sequence |
| 5 | tde1 | Mouse | Full Length |
| 6 | TDE1 | Mouse | Amino Acid |
| 7 | tde1 | Human | Coding Sequence |
| 8 | tde1 | Human | Full Length |
| 9 | TDE1 | Human | Amino Acid |
| 10 | ptv | zebrafish | Coding Sequence |
| 11 | ptv | zebrafish | Full Length |
| 12 | PTV | zebrafish | Amino Acid |
| 13 | ptv | Mouse | Coding Sequence |
| 14 | ptv | Mouse | Full Length |
| 15 | PTV | Mouse | Amino Acid |
| 16 | ptv | Human | Coding Sequence |
| 17 | ptv | Human | Full Length |
| 18 | PTV | Human | Amino Acid |
| 19 | moesin | zebrafish | Coding Sequence |
| 20 | moesin | zebrafish | Full Length |
| 21 | Moesin | zebrafish | Amino Acid |
| 22 | moesin | Mouse | Coding Sequence |
| 23 | moesin | Mouse | Full Length |
| 24 | Moesin | Mouse | Amino Acid |
| 25 | moesin | Human | Coding Sequence |
| 26 | moesin | Human | Full Length |
| 27 | Moesin | Human | Amino Acid |
| 28 | hke4 | zebrafish | Coding Sequence |
| 29 | hke4 | zebrafish | Full Length |
| 30 | HKE4 | zebrafish | Amino Acid |
| 31 | hke4 | Mouse | Coding Sequence |
| 32 | hke4 | Mouse | Full Length |
| 33 | HKE4 | Mouse | Amino Acid |
| 34 | hke4 | Human | Coding Sequence |
| 35 | hke4 | Human | Full Length |
| 36 | HKE4 | Human | Amino Acid |

This application provides nucleic acid and polypeptide sequences that include those shown in Table 1. Protein names are generally set forth herein in capital letters while nucleic acid sequence names are set forth in lower case; however, this usage is for the sake of convenience, and embodiments that apply to proteins are intended to apply to nucleic acid sequences, and vice versa, as is appropriate in that context. The nucleic acid sequences set forth herein are intended to represent both DNA and RNA sequences, according to the conventional practice of allowing the abbreviation “T” stand for “T” or for “U”, as the case may be, for DNA or RNA. Note that full-length sequences contain untranslated portions while coding sequences start at the start codon and end at the stop codon.

One embodiment is an isolated nucleic acid comprising a sequence that hybridizes under stringent conditions to a hybridization probe, wherein the probe is a member of the group consisting of a coding sequence for TDE1, PTV, MOESIN, or HKE4; or wherein the probe is a complement thereof. The isolated sequence may have, for example, phosphorothioate or morpholino phosphorodiamidate components. The isolated sequence may have, for example, sequences that are at least partially identical, e.g., 80% or 90% identical to the TDE1, PTV, MOESIN, or HKE4 sequence.

Another embodiment is a composition having an isolated polypeptide comprising an amino acid sequence that is at least 8 residues in length and is at least 80% or 90% identical to at least a portion of a polypeptide sequence for TDE1, PTV, MOESIN, or HKE4. Another embodiment is an antisense polynucleic acid having a sequence, wherein the antisense polynucleic acid suppresses the expression of a polypeptide at least 80% or 90% identical to at least a portion of a polypeptide sequence for TDE1, PTV, MOESIN, or HKE4.

Another embodiment is a vector, the vector having a first nucleic acid sequence that hybridizes under stringent conditions to a second nucleic acid sequence, wherein the second sequence is a coding or full length nucleic acid sequence for TDE1, PTV, MOESIN, or HKE4. Another embodiment is a vertebrate nonhuman animal comprising such a vector. Examples of animals include humans, zebrafish, mouse, rat, sheep, pigs, horses, bonobos, simians, monkeys, and goats.

Another embodiment is a method of using a composition, the method including administering a composition to an animal, the composition comprising a polypeptide having an amino acid sequence that is at least 80% or 90% identical to at least a portion of a polypeptide sequence for TDE1, PTV, MOESIN, or HKE4.

Another embodiment is a method that includes administering a vector to an animal, the vector including a first nucleic acid sequence that hybridizes under stringent conditions to a second nucleic acid sequence, wherein the second sequence is a member of the group consisting of sequences that are at least 70%, 80%, or 90% identical to a TDE1, PTV, MOESIN, or HKE4 sequence for a polypeptide or nucleic acid.

Another embodiment is a screening that includes providing a polypeptide having an amino acid sequence that is at least 70%, 80%, or 90% identical to TDE 1, PTV, MOESIN, or HKE4; exposing the polypeptide to a factor; and determining that the factor has a specific binding affinity for the polypeptide. The factor may be, for example, provided in isolated form by using a separations process that separates the bound factor from the polypeptide. Another embodiment is a method of administering a compound, the method comprising preparing a composition of a factor, wherein the factor is isolated as described herein.

Another embodiment is composition, the composition comprising a combination of a pharmaceutically acceptable carrier, VEGF, and TDE1, PTV, or MOESIN. Another embodiment is a composition having a factor that is a derivative, mimic, imitator, agonist, or antagonist, of VEGF in combination with VEGF, TDE 1, PTV, or MOESIN.

Another embodiment is a method of using a composition, the method comprising administering the composition to an animal, the composition comprising a polypeptide having an amino acid sequence that is at least 70%, 80%, or 90% identical to TDE1, PTV, MOESIN, or HKE4. Such compositions may be administered, for example, into or near a tumor, into or near a heart, into or near a necrotic tissue, and into or near diabetic or venous ulcer. Alternatively, the compositions may be administered locally or systemically.

### BRIEF DESCRIPTION OF THE FIGURES

FIG. 1 is an alignment of TDE1 and TDE2 (also called TMS2) proteins for Danio rerio, Homo Sapiens, and Mus Musculus;

FIG. 2 shows the percent similarity between zebrafish TDE1 (zfTDE1), human TDE1 (hTMS1), mouse TDE1 (mTMS1), human TDE2 (hTMS2), and mouse TDE2 (TMS2).

FIG. 3 is a bar graph that shows microangiography results fro zebrafish embryos treated with antisense against TDE1;

FIG. 4 is a bar graph that shows microangiography results fro zebrafish embryos treated with antisense against TDE1 and/or VEGF;

FIG. 5 is a bar graph that shows results for angiogenesis markers in zebrafish treated with TDE1 antisense;

FIG. 6 is an alignment of PTV proteins for Danio rerio, Homo Sapiens, Mus Musculus;

FIG. 7 shows the percent similarity for PTV between species: Homo sapiens, Mus musculus, Rattus norvegicus, Danio rerio, Drosophila melanogaster, Caenorhabditis elegans, Schizosaccharomyces pombe, and Arabidopsis thaliana;

FIG. 8 is a bar graph that shows microangiography results fro zebrafish embryos treated with antisense against PTV;

FIG. 9 is a bar graph that shows microangiography results fro zebrafish embryos treated with antisense against PTV and/or VEGF;

FIG. 10 is a bar graph that shows results for angiogenesis markers in zebrafish treated with PTV antisense;

FIG. 11 is an alignment of MSN proteins for Danio rerio, Homo Sapiens, and Mus Musculus;

FIG. 12 is a percent similarity table that shows similarity results for MSN and ERM family members in human, mouse, and zebrafish;

FIG. 13 is a bar graph that shows microangiography results fro zebrafish embryos treated with antisense against MSN;

FIG. 14 is a bar graph that shows results for angiogenesis markers in zebrafish treated with MSN antisense;

FIG. 15 is an alignment of HKE4 proteins for Danio rerio, Homo Sapiens, and Mus Musculus;

FIG. 16 is a percent similarity table that shows the percent similarity for HKE4 between species: Homo sapiens, Mus musculus, Danio rerio, Drosophila melanogaster, and Arabidopsis thaliana;

FIG. 17 is a bar graph showing results for Alcian blue staining of hke4-antisense injected zebrafish embryos;

FIG. 18 is a bar graph showing results for calcein staining of hke4-antisense injected zebrafish embryos.

### DETAILED DESCRIPTION

This disclosure describes certain molecules such as nucleic acid and protein sequences and provides data for understanding their biological functions. Also set forth are compositions that include these molecules, as well as methods for using them. Genes and associated molecules are described, with the orthologs and homologues in the human, mouse, and zebrafish being set forth. In general, the human, mouse, and zebrafish polypeptide orthologs and homologues are expected to have very similar structures and functions, based on their expression patterns, sequence homology, and good correlation observed between the three models. The names for the genes herein are: testicular tumor differentially expressed (tde1, also referred to as tms1); Patchy Vessels (ptv); moesin, and HLA class II region expressed gene 4 (hke4). One step described herein for the discovery of the sequences and their function involved use of morpholino antisense molecules in zebrafish embryos. In brief, zebrafish embryos were injected at an early embryonic stage with morpholino antisense molecules. Observations of the resultant phenotype and localization of the antisense molecules with the target mRNA provided data as to the function of the nucleic acids that interacted with the antisense molecules. These and certain other data were used to identify the structure of messenger ribonucleic acid (mRNA) sequences, their deoxynucleic acid sequences (DNA), their amino acid sequences, and their function.

The TDE Family of Membrane Polypeptides

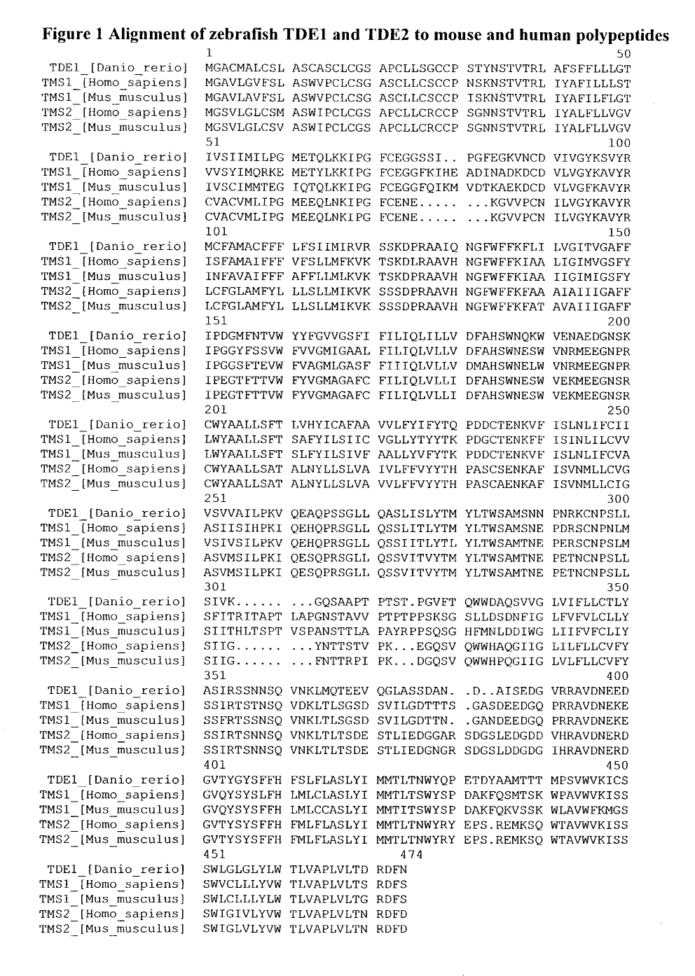

The TDE family (alternately called TMS, with TMS and TDE being used interchangeably herein) of proteins are a group of similar transmembrane proteins of previously unknown function that appear conserved among eukaryotes (Hill, K. K. 1995; Krueger, W. H. H. 1997; Nelissen, B. 1997). The proteins in this family are predicted to have 11 transmembrane domains and a conserved myc-type ‘helix-loop-helix’ dimerization domain signature (Grossman, T. R. 2000; Suzuki, M. 1998). They also share a series of 4-6 conserved cysteines within the amino-terminal 30 amino acids (see alignment FIG. 1).

In general, determinations of family membership or classification of a polypeptide can involve comparison of structural features and sequence identities. Thus, an aspect for identification of a newly identified polypeptide as belonging to the TDE family of polypeptides is to determine if the newly identified polypeptide has characteristic conserved domains described above. Additionally, another aspect of identification of a new polypeptide as belonging to the TDE family of polypeptides is by amino acid or nucleic acid sequence comparison with known TDE polypeptides. For example, a newly identified polypeptide can be classified as belonging to the TDE family of polypeptides if the newly identified polypeptide is more similar to any member of the TDE family of polypeptides than the two least similar members within the TDE family (see FIG. 2). Examples of polypeptides belonging to the TDE family include two human and two mouse TDE homologues (human accession number AAB48858 (Bossolasco, M. 1999); mouse accession number AAD54420 (Lebel, M. 1994)) and TMS2 (Grossman, T. R. 2000) (human accession number AAH33029; mouse accession number AAA74236).

A person of ordinary skill in the art will be able to determine if a new polypeptide is a member of the TDE family based on an aspect discussed above, or a combination of them. Since the classification or function of a biomolecule is related to its sequence, it is expected that a given polypeptide can be considered to be like TDE1 when the given polypeptide is determined to be a member of the TDE family and has a percent identity that is closer to TDE1 than to other TDE family members. Example 2 sets forth the procedure used to identify the sequences.

Studies of TDE1 expression (see Example 3) showed that expression of TDE1 in zebrafish embryo are similar to patterns of TDE1 expression in adult mouse that are published elsewhere (Grossman, T. R. 2000). Since these expression patterns are similar, it can be expected that functional aspects of TDE1 in zebrafish embryos will correlate to the functional aspects of TDE1 in the mouse and human. This is because gene function in zebrafish mirrors that in mammals, including humans (Clark, M. D. 2001, “An oligonucleotide fingerprint normalized and expressed sequence tag characterized zebrafish cDNA library.” Genome Res 11: 1594-602; Dodd A, Curtis P M, Williams L C, Love D R. (2000); and Sumanas, S. and Larson, J 2002).

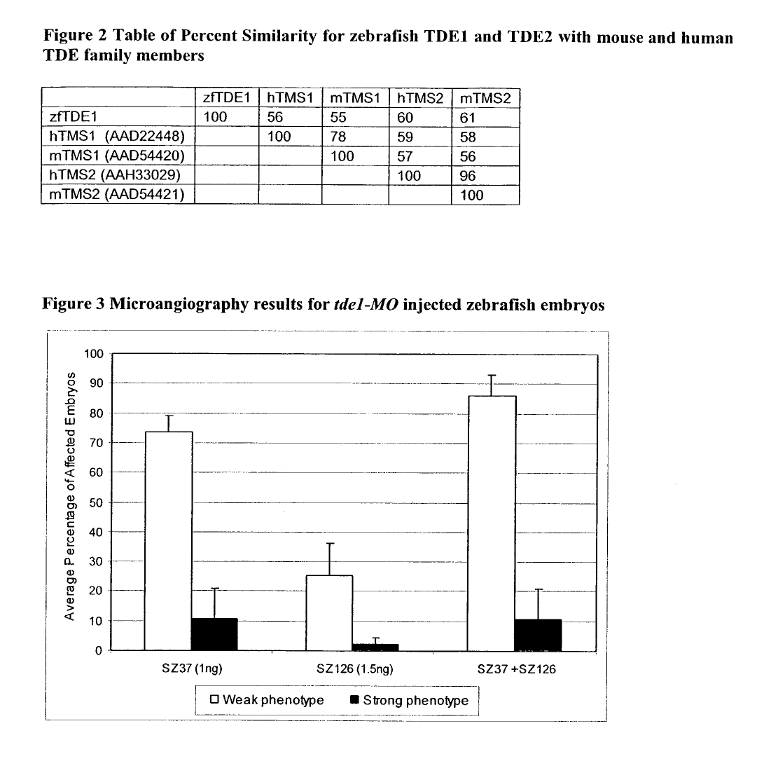

Observations of zebrafish embryos after injection with specific morpholino antisense molecule(s) indicated that TDE1 had a significant function and in situ hybridization experiments show expression in at least some specific tissues (Examples 3 and 4). Microangiography showed that inhibition of TDE1 blocks blood vessel formation: more than half of the tested animals exhibited decreased or no blood vessel formation subsequent to antisense inhibition of TDE1 using certain antisense sequence(s), as shown in FIG. 3 (Example 6). Antisense molecules were shown to predictably induce a phenotypic response, either alone or in combination.

Example 7 describes results of in situ hybridization analysis of VE-cadherin (cdh5) which showed that TDE1 preferentially inhibited angiogenesis as compared to vasculogenesis. Moreover, FIG. 5 (Example 9) shows that inhibition of tde1 also disrupts the expression of known vascular endothelial cell markers in the intersomitic vessels. This also confirms that tde1 was involved in blood vessel formation and its inhibition preferentially inhibited angiogenesis as compared to vasculogenesis.

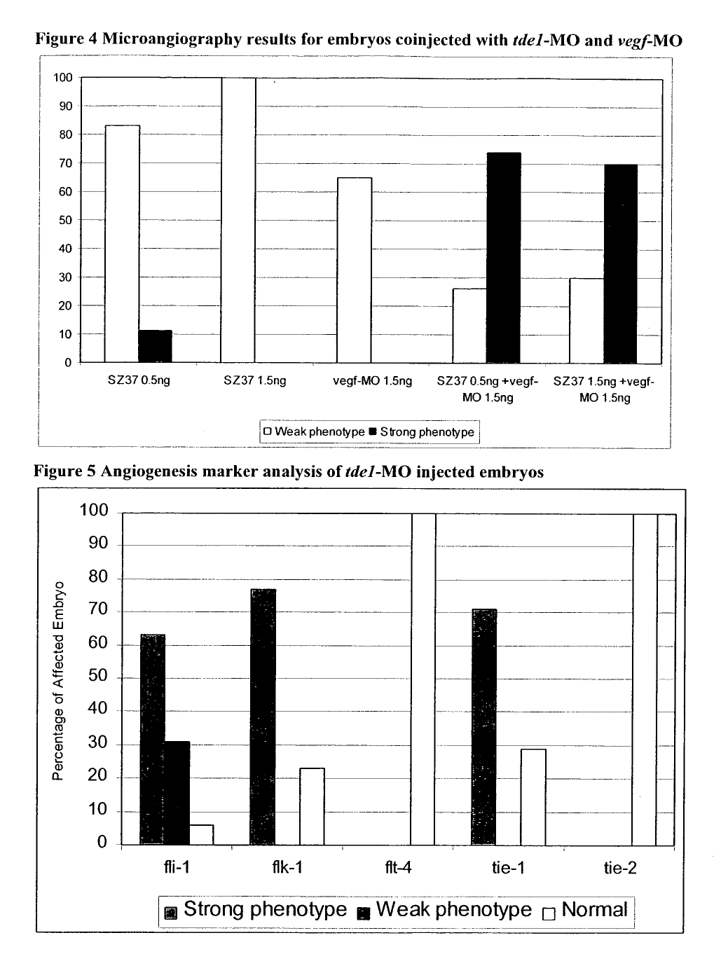

Example 8 shows that TDE1 imitates the function and synergizes in function with vascular endothelial growth factor (VEGF). Inhibition of both tde1 and vegf produced a synergistic effect for the inhibition of blood vessel formation (FIG. 4). VEGF is known to be active in blood vessel formation in humans. The evidence that TDE1 cooperates with VEGF indicates that TDE1 is like VEGF in that it is also active in blood vessel formation.

Further, the zebrafish embryonic model is predictive for human function because the zebrafish model predicted that TDE1 would be involved in blood vessel formation and, in fact, TDE1 was determined to be synergistic with VEGF. VEGF is well established to affect pathways involved in blood vessel formation in humans. Since VEGF synergizes with TDE1, VEGF and TDE1 can affect some of the same pathways. Since TDE1 and VEGF affect the same pathways, and the pathways are implicated in blood vessel formation in humans, it is expected that TDE1 will affect blood vessel formation in humans.

Further, since VEGF is synergistic with TDE1, it is reasonably expected that a use of VEGF, an agent that promotes VEGF activity, or an agent that inhibits VEGF activity will benefit from the alternative use of, or the combination of use with TDE1, an agent that promotes TDE1 activity, or an agent that inhibits TDE1 activity. Uses for VEGF, derivatives of VEGF, inhibitors thereof, and mimics and imitators thereof include those set forth in U.S. Pat. Nos. 6,262,337; 6,451,342; and 6,284,751, and in U.S. patent application Pub. Nos. 20030008824; 20020192634; 20020165174; 20020132978; 20020051965; 20020065218; and 20020037544. In this general context, activity means the action that the molecule or agent produces: for example, VEGF has an action of stimulating human blood vessel formation, so blood vessel formation is an activity of VEGF. Promotion of VEGF activity means materials and methods that mimic or imitate VEGF activity: for example, a portion of VEGF that promotes a blood vessel formation activity of VEGF could be used to promote VEGF activity in, for example, a wound, a tissue, or a cell culture. Inhibiting an activity means a material or method that reduces or essentially or completely blocks an activity: for example, a ligand that binds VEGF and competitively inhibits the binding of VEGF to a cell receptor so that the second messengers normally triggered by the cell receptor binding of VEGF are not triggered. A mimic is a compound that has essentially the same effect as another compound. An imitator is a compound that has a similar function as another compound but not an identical function, so that there may be differences in potency, additional functions, or additional molecular structure.

Moreover, TDE1 is expected to be useful in wound healing applications because promotion of its function will stimulate blood vessel formation. Blood vessel formation is generally an important aspect of wound healing. Wounds that are recalcitrant to blood vessel formation or healing are expected to particularly benefit from TDE1.

Previous studies have shown TDE1 expression in mouse liver tumor (Grossman, T. R. 2000) and in mouse testicular tumors (Lebel, M. 1994) but these studies do not link TDE1 to angiogenesis. The published studies referenced herein that show TDE1 in tumors do not suggest a function or purpose for TDE1 because the studies do not show a link between polynucleic acid or polypeptide expression and any of the many potential aspects of tumor activity, e.g., as related to oncogenes, immune system function, control of differentiation, or transcription regulation.

Patchy Vessels (PTV): a 30 kDa Protein.

PTV is a 30 kDa protein with homologues found in humans, mouse, and zebrafish. Its structure indicates that it is a transmembrane protein and it appears to be widely biologically conserved. The data indicates that it is a member of a group of proteins having 3 transmembrane domains. Examples include human and mouse homologues found in public databases of sequencing data: LOC55831 (human accession number AAF67487) 0610039A15Rik (mouse accession number AAH04641). Example 10 sets forth procedures used for isolating ptv. Expression patterns for ptv are set forth in Example 11.

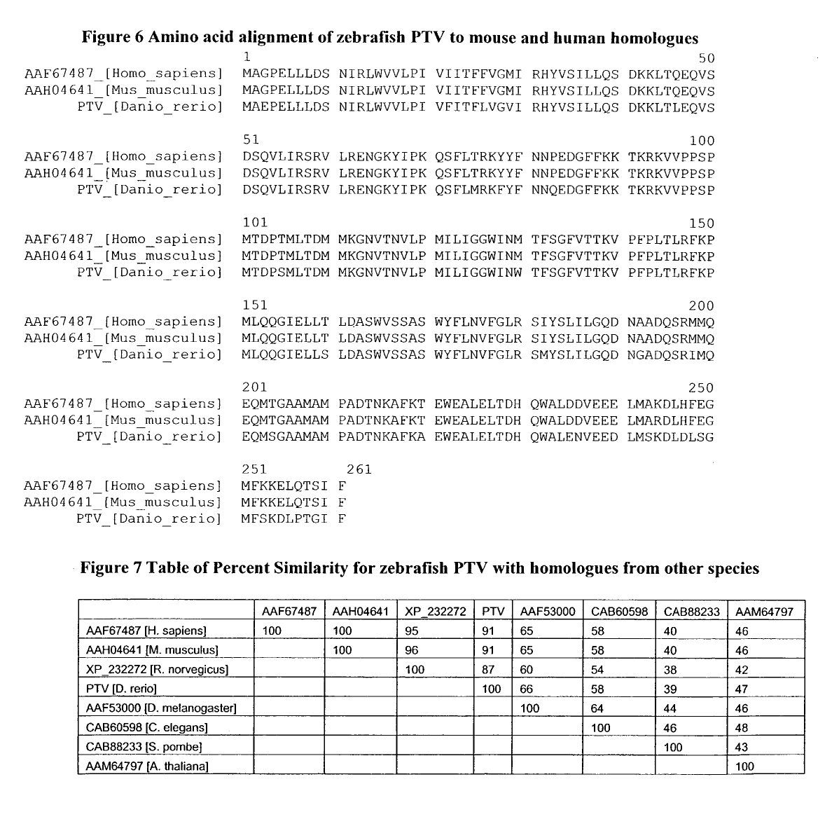

FIG. 6 (see Example 10) sets forth an alignment of PTV showing a comparison of zebrafish, mouse, and human homologues, whereby new homologues of PTV may be identified. FIG. 7 shows the percent similarity between zebrafish PTV and homologues from other species. PTV is significantly conserved between mammals and other vertebrates, as shown in FIG. 7 wherein human (Homo sapiens) PTV has essentially 100% similarity with mouse (Mus musculus), 95% similarity with rat (Rattus norvegicus), and 91% similarity with zebrafish (Danio rerio).

Persons of ordinary skill in these arts will be able to determine if a given polypeptide or polynucleic acid is a new homologue of PTV. In general, such determinations typically involve comparison of structural features and sequence identities. Any new homologue of PTV would be expected to not be identical to the PTV members disclosed herein and would therefore have a certain percent identity with the PTV sequences that is less than 100%. Therefore, it would be possible to determine the percent identity of a given sequence with PTV as disclosed herein and any new homologues of PTV. In general, a newly identified polypeptide can be classified as a homologue of PTV if the newly identified polypeptide is more similar to a member of the known PTV polypeptides than the two least similar members within the group of PTV homologues (see FIG. 7). Moreover, structural aspects of the newly identified polypeptide are typically useful in such a determination. Since the classification or function of a polymeric biomolecule is related to its sequence, it is expected that a given polypeptide can be considered to be like a certain PTV when the given polypeptide is determined to be a homologue of PTV and has a percent identity that is closer to the PTV member disclosed herein than to other unrelated sequences.

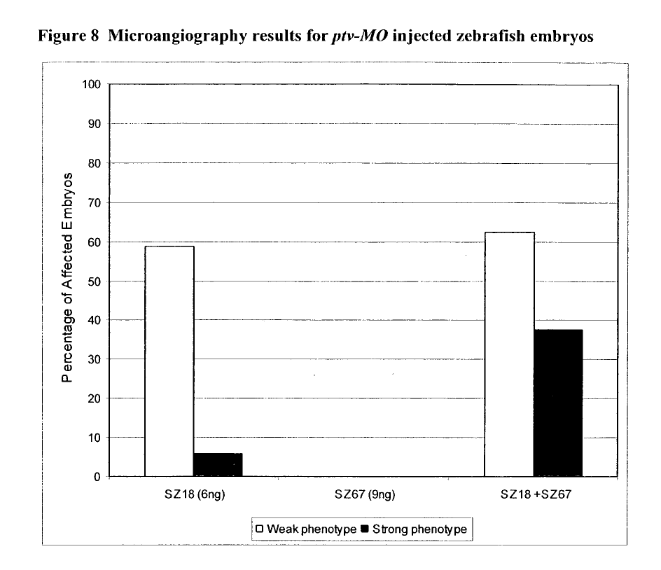

Morpholino injections (Example 12) were used in certain studies of PTV. Antisense molecules against ptv were injected into zebrafish embryos with the result that essentially normal phenotypes were observed using microscopy (Example 13). Microangiography analysis of the vasculature, however, showed that ptv was involved in blood vessel formation and was preferential for angiogenesis compared to vasculogenesis (FIG. 8, Example 14). Expression of cdh5 confirmed that ptv was involved in blood vessel formation and its inhibition preferentially inhibited angiogenesis as compared to vasculogenesis (Example 15).

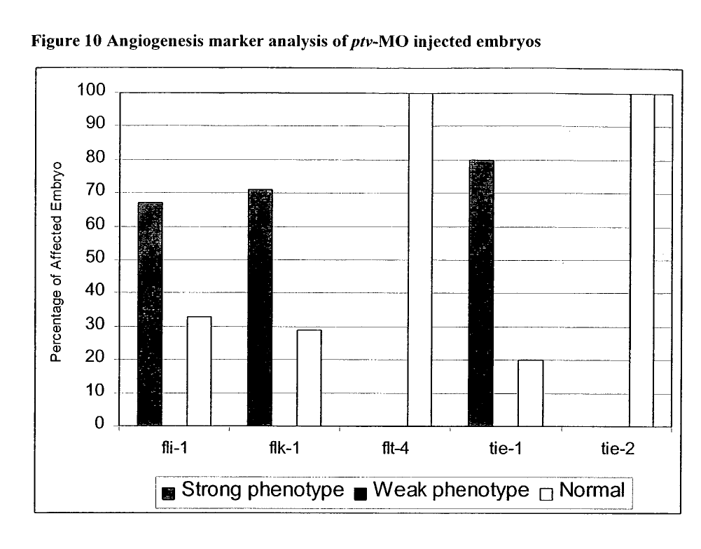

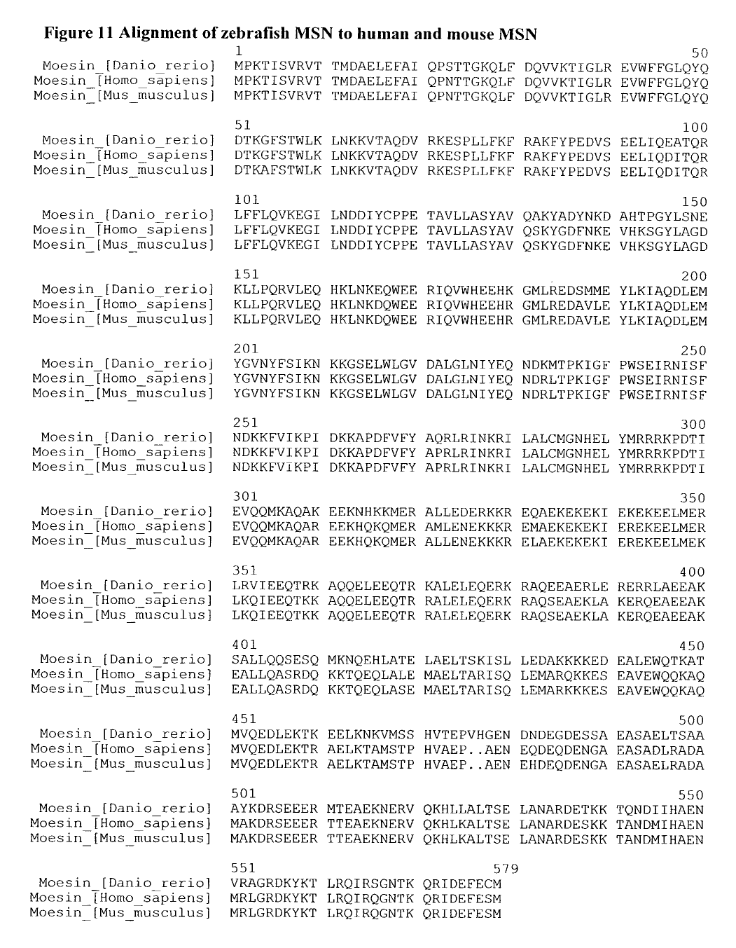

Moreover, FIG. 10 (Example 17) shows that inhibition of ptv also disrupts the expression of known vascular endothelial cell markers in the intersomitic vessels. This also confirms that ptv was involved in blood vessel formation and its inhibition preferentially inhibited angiogenesis as compared to vasculogenesis.

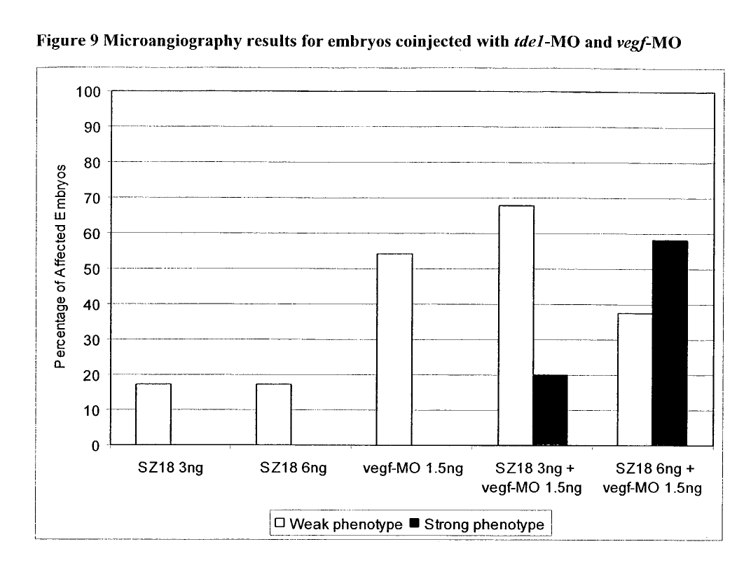

Example 16 shows how PTV imitates and synergizes with vascular endothelial growth factor (VEGF). Inhibition of both ptv and vegf produced a synergistic effect for the inhibition of blood vessel formation (FIG. 9). This evidence that PTV cooperates with VEGF indicates that PTV is like VEGF in that it is also active in blood vessel formation. Further, it is expected that promoting or reducing activities of both ptv and vegf would have a more powerful effect than promoting or reducing activities of vegf alone. Thus an agent that promotes vegf activity would be expected to be more effective when used in conjunction with an agent that also promotes ptv activity. Many agents and techniques for the promotion of reduction of activities of vegf are known to artisans of ordinary skill; similarly, many agents and techniques for promoting or reducing activities of ptv will be understood by artisans of ordinary skill after reading this disclosure.

Further, the zebrafish embryonic model is predictive for human function because the zebrafish model predicted that PTV would be involved in blood vessel formation and, in fact, PTV was determined to be synergistic with VEGF. VEGF is well established to be involved to affect pathways involved in blood vessel formation in humans. Since VEGF synergizes with PTV, VEGF and PTV are expected to affect some of the same pathways in zebrafish, humans, mammals, and vertebrates. Since PTV and VEGF affect the same pathways, and the pathways are implicated in blood vessel formation in humans, it is expected that PTV will affect blood vessel formation in humans.

Further, since VEGF is synergistic with PTV, it is reasonably expected that a use of vegf, an agent that promotes vegf activity, or an agent that inhibits vegf activity will benefit from the alternative use of, or the combination of, use of with ptv, an agent that promotes ptv activity, or an agent that inhibits ptv activity. Moreover, PTV is expected to be useful in wound healing and restoration of other necrotic tissues, such as venous and diabetic ulcers as well as ischemic heart tissue, applications because promotion of its function will stimulate blood vessel formation. Blood vessel formation is generally an important aspect of wound healing. Wounds that are recalcitrant to blood vessel formation or healing are expected to particularly benefit from PTV

The ERM Family of Polypeptides

ERM refers to polypeptides belonging to the Ezrin/radixin/moesin family of proteins. These membrane-associated proteins form a linkage between filamentous actin in the cell cortex and membrane proteins on the cell surface (Sato, N. 1992). Polypeptides classified as belonging to the ERM family of polypeptides have the following characteristic conserved motifs in their amino acid sequences: FERM domain Band 4.1 superfamily (Pfam reference PF00323) and ERM family domain (Pfam reference PF00769 1996-2002). Examples of polypeptides belonging to the ERM family include four human and four mouse ERM homologues:

Ezrin (human accession number AAH13903 (Gould, K. L. 1989; Turunen O. 1989); mouse accession number CAA43086 (Funayama, N. 1991))

Radixin (human accession number AAA36541 (Wilgenbus, K. K. 1993); mouse accession number CAA43087 (Funayama, N. 1991))

Moesin (human accession number AAA36322 (Lankes, W. T. 1991); mouse accession number P26041 (Sato, N. 1992)

Merlin (human accession number AAA36212 (Rouleau, G. A. 1993; Trofatter, J. A. 1993); mouse accession number AAA63648 (Haase, V. H. 1994; Huynh, D. P. 1994))

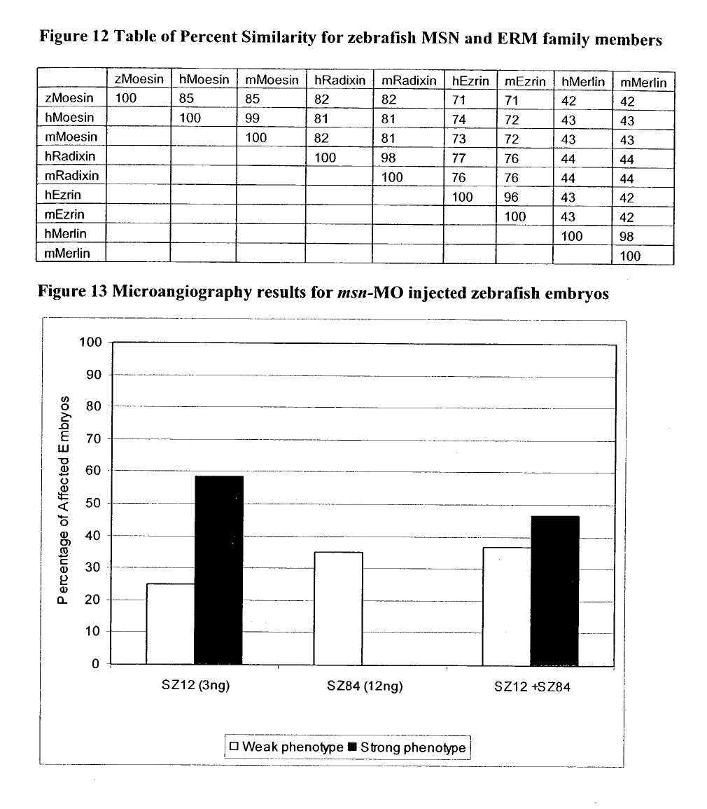

Both Ezrin and Radixin contain a characteristic poly-proline stretch in the carboxy-terminal portion of the amino acid sequence that is lacking in both Moesin and Merlin (see alignment FIG. 11). Merlin is the most divergent member of the ERM family identified so far and contains a longer amino-terminal sequence than the other three proteins (see FIG. 12).

A new polypeptide can be identified as belonging to the ERM family of polypeptides by amino acid or nucleic acid sequence comparison with known ERM polypeptides. In general, determinations of family membership or classification of a polypeptide typically involve comparison of structural features and sequence identities. Thus, an aspect for identification of a newly identified polypeptide as belonging to the ERM family of polypeptides is to examine if the newly identified polypeptide has most of the characteristic features described above, e.g., the characteristic conserved motifs, the poly-proline stretch in the carboxy-terminal portion, or a longer amino-terminal sequence like Merlin. Additionally, another aspect of identification of a new polypeptide as belonging to the ERM family of polypeptides is by amino acid sequence comparison with known ERM polypeptides. For example, a newly identified polypeptide can be classified as belonging to the ERM family of polypeptides if the newly identified polypeptide is more similar to any member of the ERM family of polypeptides than the two least similar members within the ERM family (see FIG. 12).

A person of ordinary skill in the art will therefore be able to determine if a polypeptide is a member of the ERM family based an aspect discussed above, or a combination of them. Since the classification or function of a biomolecule is related to its sequence, it is expected that a given polypeptide can be considered to be like a certain MOESIN when the given polypeptide is determined to be a member of the ERM family and has a percent identity that is closer to MOESIN than to other ERM family members.

Spatial expression patterns of MOESIN in zebrafish embryos were shown to be consistent with expression patterns for mouse embryos and these patterns are consistent with expression of MOESIN in adult mouse (Berryman, M. 1993) and adult human (Johnson, M. W. 2002) tissues as previously published (Example 19). These facts indicate that the zebrafish embryo model is predictive for function in other animals, including human. A phenotype indicative of a MOESIN role in vascular formation was identified using morpholino antisense molecules in zebrafish embryos (Example 21). Microangiography (FIG. 13, Example 22) and studies of cdh5 expression (Example 23) supported this indication for a role in vascular formation. The microangiography and cdh5 expression studies showed that the vascular activity in MOESIN exhibited specificity for angiogenesis as compared to vasculogenesis.

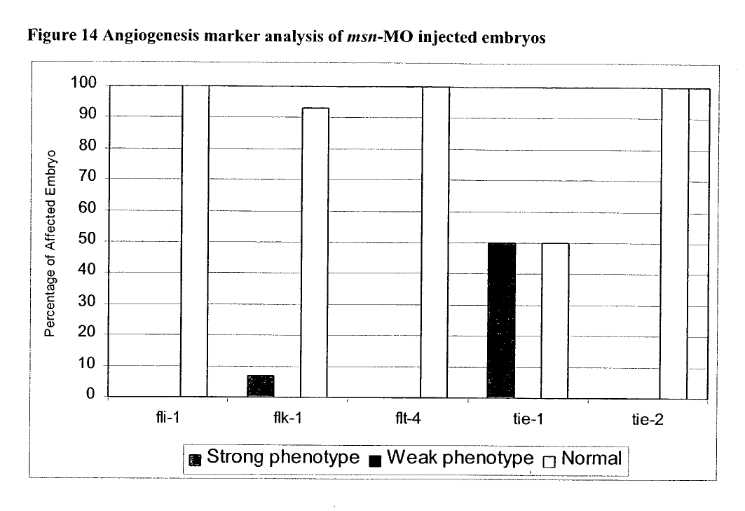

Example 24 (FIG. 14) shows the results of studies of early and late vascular markers. These studies also indicated a role for MOESIN in angiogenesis. Therefore, MOESIN is expected to have applications to disease states related to angiogenesis including but not limited to cancer, ischemia and wound healing.

A potential role for moesin in vascular formation was not established from the knockout of moesin in mice (Yoshinori, D. 1999) in that mice developed normally. This result is consistent with antisense studies of mouse tissue culture cells where functional redundancy was observed among the ezrin, radixin and moesin proteins (Takeuchi, K. 1994). We believe that similar tests have not been carried out in humans. The studies in zebrafish outlined herein clearly show a role for moesin in angiogenesis. This phenotype may have been masked by functional redundancy in normal mouse development. However, the relevance of this functional redundancy to the role that angiogenesis plays in disease states is not described. Therefore, since MOESIN affected vascular activities in the studies reported herein, it is expected that MOESIN activity will have effects on the vascular system of humans. It is reasonably expected that a use of MOESIN, an agent that promotes moesin-like activity, or an agent that inhibits moesin-like activity will be effective in such applications.

The HKE4 Proteins

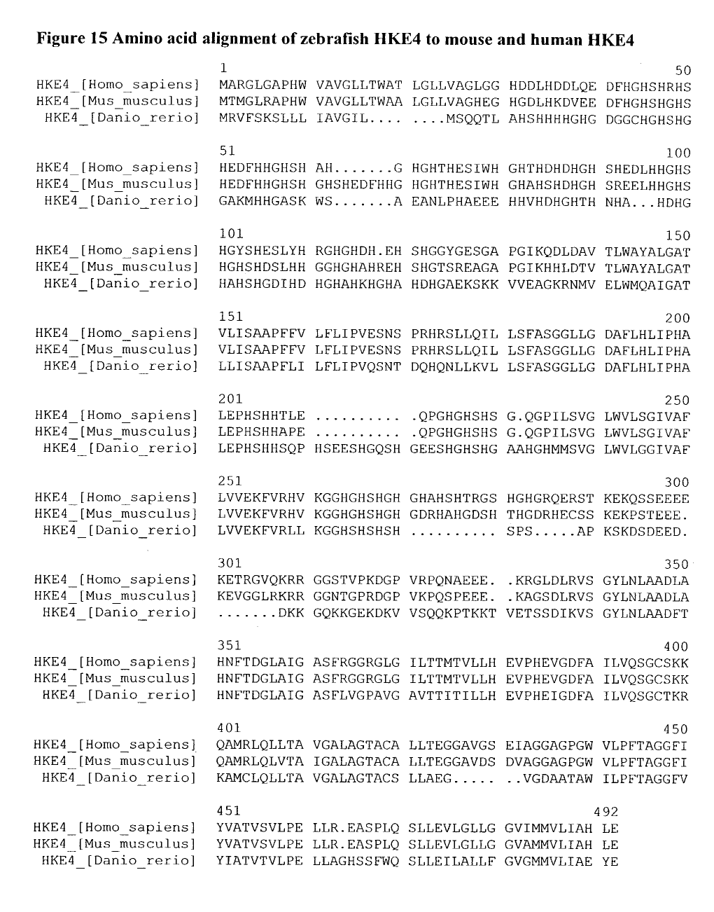

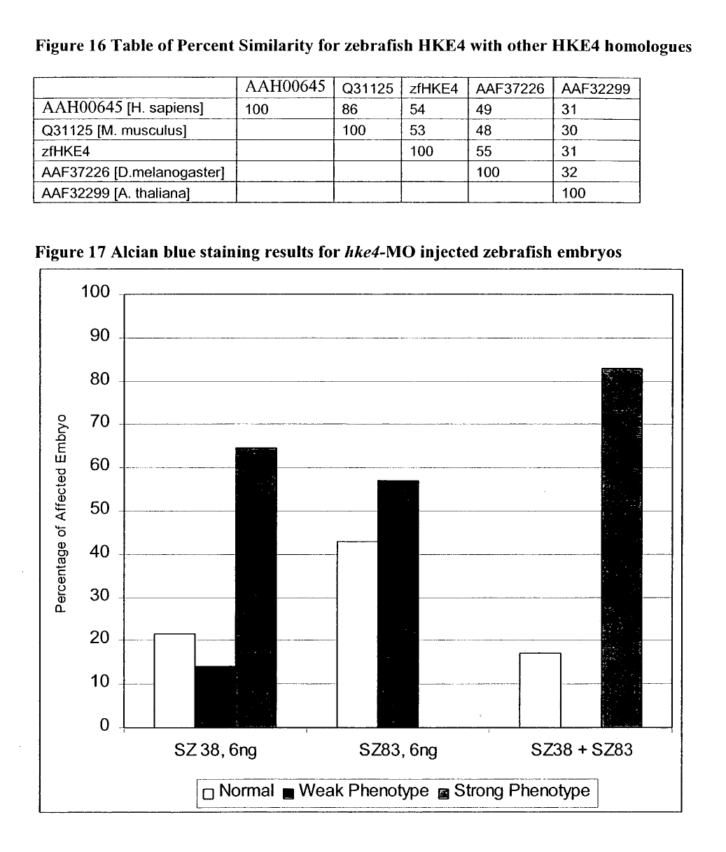

The HKE4 protein is a protein of previously unknown function that appears widely biologically conserved based on comparisons to other family members and is apparently a transmembrane protein (Suzuki, A. 2002; Lasswell, J. 2000). FIG. 15 (Example 25) shows a comparison of HKE4 to other members of the HKE4 family. FIG. 16 shows a percent amino acid sequence identity for HKE4 and other family members. This group of proteins is predicted to have a signal sequence and 7 transmembrane domains. The proteins also contain histidine rich tracts and a ZIP zinc transporter domain (Pfam reference PF02535 1996-2002). Examples include human and mouse HKE4 homologues: HKE4 (human accession number AAH00645 (Janatipour, M. 1992; Ando, A. 1996); mouse accession number Q31125 (Abe, K. 1988; St.-Jaques, B.1990)); CATSUP (accession number AAF37226 [D. melanogaster] (Stathakis, D. G. 1999)); and IAR1 (accession number AAF32299 [A. thaliana](Laswell, J. 2000))

In general, determinations of family membership or classification of a polypeptide typically involve comparison of structural features and sequence identities. Thus, an aspect for identification of a newly identified polypeptide as belonging to the HKE4 family of polypeptides is to determine if the newly identified polypeptide has characteristic conserved domains described above. Additionally, another aspect of identification of a new polypeptide as belonging to the HKE4 family of polypeptides is by amino acid or nucleic acid sequence comparison with known HKE4 polypeptides. For example, a newly identified polypeptide can be classified as belonging to the HKE4 family of polypeptides if the newly identified polypeptide is more similar to any member of the HKE4 family of polypeptides than the two least similar members within the HKE4 family (see FIG. 16). A new polypeptide can be identified as a HKE4 homologue by amino acid or nucleic acid sequence comparison with known HKE4 polypeptides.

A person of ordinary skill in the art will be able to determine if a polypeptide is a member of the HKE4 family based on an aspect discussed above, or a combination of them. Since the classification or function of a biomolecule is related to its sequence, it is expected that a given polypeptide can be considered to be like a certain HKE4 polypeptide or polynucleic acid set forth herein when the given polypeptide is determined to be a member of the HKE4 family and has a percent identity that is closer to HKE4 than to other HKE4 family members.

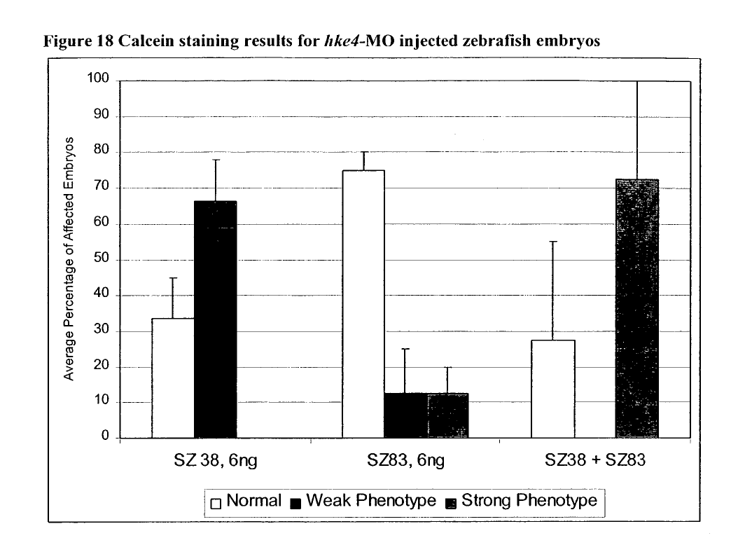

Example 25 sets forth the procedure used to identify the HKE4 sequences. Studies of HKE4 expression (see Examples 26 and 27) showed that expression of HKE4 was preferentially expressed in certain tissues. Morpholino antisense injection (Example 28) caused defects in cartilage formation (Example 29). Alcian blue staining for visualization of cartilage showed further aspects of cartilage defects caused by HKE4 inhibition, and certain antisense sequences were identified as being synergistic (FIG. 17, Example 29). HKE4 was determined to have relatively little effect on neural crest formation (Example 30). Calcein staining showed bone defects caused by HKE4 inhibition (FIG. 18, Example 31).

These results showed that HKE4 has activity for cartilage and bone formation. These two activities are often linked because cartilage formation is involved in many aspects of bone formation. Previous studies by others have implicated HKE4 in ionic transport (Laswell, J. 2000; Suzuki, A. 2002). However, identification as a potential ionic transporter does not suggest a role in bone or cartilage formation because ionic transporters are found throughout the body and have many roles.

Since HKE4 affected bone and cartilage activities in the studies reported herein, it is expected to be have effects on the bone and cartilage system of humans, for example, remodeling, growth, stimulation, organizing, structuring. It is reasonably expected that a use of HKE4, an agent that promotes hke4 activity, or an agent that inhibits hke4 activity will be effective in such applications. Indeed, HKE4 is well conserved and, besides being functional in zebrafish has disclosed herein, is functional in Drosophila, mouse, and Arabadopsis thaliana (Laswell, J. 2000; Stathakis, D. G. 1999). For example, the mouse homologue of HKE4 (AAA37767) has been shown to functionally substitute in vivo for a more distantly related plant homologue of HKE4 in Arabadopsis thaliana (accession number AAF32299) (Laswell, J. 2000). Therefore it is reasonably expected to be functional in humans. It has not been previously understood, however, that HKE4 is involved in cartilage and bone functions, e.g. remodeling, growth, stimulation, organizing, structuring.

Zebrafish Embryo Assays

A suitable system that is useful for determining function or phenotype associated with a selected nucleic acid of known sequence is the morpholino-modified polynucleotide analogue/zebrafish system. The system involves delivery of morpholinos to zebrafish, e.g., by microinjection or merely exposing the model organism to the polynucleotide analogue. This approach makes morpholino targeting highly predictable for polynucleotide design and significantly reduces non-specific effects. In contrast, more traditional antisense polynucleotide approaches have used RNase-H-based degradation of mRNA as a mechanism of action. Detailed aspects of such systems and examples of their employment are set forth, forth example, in U.S. patent applications Ser. No. 09/918242, filed Jul. 30, 2001 entitled “Inhibition Of Gene Expression Using Polynucleotide Analogues”; Ser. No. ______ filed Jan. 17, 2003 entitled “Syndecans and Angiogenesis; and PCT WO03004610, entitled “HSST and Angiogenesis”, filed Jul. 3, 2002. And also in: Sumanas, S. and J. Larson (2002), “Morpholino phosphorodiamidate oligonucleotides in zebrafish: a recipe for functional genomics?” Briefings in Functional Genomics and Proteomics 1: 239-256; Dodd, A., P. M. Curtis, L. C. Williams and D. R. Love (2002) “Zebrafish: bridging the gap between development and disease” Hum. Mol. Genet. 9: 2443-2449; Shin, J. T. and M. C, Fishman (2002) “From zebrafish to human: modular medical models” Ann. Rev. Genomics Hum. Genet. 3: 311-340; Sehnert, A. J. and D. Y. R. Stainier (2002) “A window to the heart: can zebrafish mutants help us understand heart disease in humans?” Trends Genet 18: 491-494. Morpholinos (morpholino-modified polynucleotide analogues) are not subjected to any known endogenous enzymatic degradation activity. Morpholinos have been shown to bind to and block translation of mRNA both in vitro and in tissue culture.

In general, morpholinos in zebrafish have been shown to be sequence specific and extremely potent in all cells for at least the first 50 hours of development in F0 zebrafish embryos as targeted gene ‘knockdown’ agents. This period in the zebrafish embryonic development includes the fundamental vertebrate processes of segmentation and organogenesis. Thus, this tool offers the opportunity to pursue sequence-specific gene targeting studies without the necessity of laborious, time consuming, and expensive F3 vertebrate genetic testing. Morpholinos, in general, have offered a high-throughput F0 vertebrate assay system for vertebrate functional genomics applications that provides information that is otherwise difficult to get and has, in the past, required more extensive and detailed animal data.

Vascular Activity, Angiogenesis, and Vasculogenesis

Under normal physiological conditions, blood vessel formation occurs under particular conditions such as in wound healing, during tissue and organ regeneration, during embryonic vasculature development, as well as in the formation of the corpus luteum, endometrium, and placenta. Excessive, insufficient, or pathological blood vessel formation, however, has been associated with a number of disease conditions. Examples of diseases associated with excessive or insufficient blood vessel formation include rheumatoid arthritis, atherosclerosis, diabetes mellitus, retinopathies, psoriasis, and retrolental fibroplasia. In addition, blood vessel formation has been identified as a critical requirement for solid tumor growth and cancer metastasis. Examples of tumor types associated with blood vessel formation include rhabdomyosarcomas, retinoblastoma, Ewing's sarcoma, neuroblastoma, osteosarcoma, hemangioma, leukemias, and neoplastic diseases of the bone marrow involving excessive proliferation of white blood cells.

Vasculogenesis is typically associated with the establishment of blood vessels whereby endothelial cells are born from progenitor cell types. Vasculogenesis refers to generation or formation of new blood vessels. The endothelial cells are important cells in blood vessel formation and ultimately line the lumen of the vessels. In contrast, angiogenesis is a process wherein new capillaries sprout from existing vessels. Thus, angiogenesis typically associated in the process for the establishment and development of tumor tissue, as well as the control of certain inflammatory conditions.

Blood vessel formation is a broad term that encompasses the various aspects of changing or creating blood vessels, e.g., one or more of angiogenesis, vasculogenesis, blood vessel size increases or decreases, endothelial cell migration, artery formation, vein formation, capillary formation. In the zebrafish model, angiogenesis and vasculogenesis are expected to be predictive for blood vessel formation in general; however, agents that have a greater specificity for some particular aspect of blood vessel formation are expected to show a greater specificity for that aspect.

Blood vessel formation is known to play an integral role in some systems of wound healing by allowing tissue generation and remodeling. The control or inhibition of blood vessel formation can be a useful tool for the control of wound healing, inflammation and solid tumor growth. Due to the association between blood vessel formation and various disease conditions, substances that have the ability to modulate blood vessel formation, e.g., angiogenesis and/or vasculogenesis, would be potentially useful treatments for these disease conditions. Thus, since TDE1, PTV, and MOESIN are factors that are involved in blood vessel formation, it is expected that factors that mimic or imitate their activity will be useful in treatments of such conditions. Moreover, TDE1 and PTV are synergistic with VEGF so TDE1, PTV, and MOESIN are expected to synergize with other agents in humans that are useful in wound healing, e.g., VEGF, the FGF family of growth factors, other growth factors, neurotrophins, and cytokines.

For example, tumors may cause a local increase in the ratio of blood vessel formation stimulators to inhibitors, which induce the formation of new blood vessels that carry oxygen and nutrients to the growing tumor. Factors previously implicated in these processes include vascular permeability factor, vascular endothelial cell growth factor, basic and acidic fibroblast growth factors, interleukin-1, hepatocyte growth/scatter factor (HGF) and others. See, e.g., O'Reilly (1997) Regulation of Angiogenesis, Goldberg & Rosen, Eds., Birkhauser Verlag, Basel, pp. 273-294. Interfering with a stimulator of blood vessel formation, therefore, can reduce the rate of tumor growth or metastasis, or possibly interfere with blood supply to the tumor and cause it to regress. It is believed that TDE1, PTV, and MOESIN are factors that are involved in blood vessel formation so that agonists, antagonists, mimic, imitators, or inhibitors of these factors may affect tumor activity. Moreover, excessive blood vessel formation also can occur during healing at the site of a surgical incision or other tissue trauma, and can result in scarring. Agents with the ability to modulate blood vessel formation therefore also would be potentially useful in treatments to prevent scarring.

Bone and Cartilage

Natural mechanisms of growth, repair and healing are similar for bone and cartilage. While these mechanisms require a series of events that are not completely understood, it is known that specific factors are required to stimulate osteoblasts and chondrocytes and odontoblasts in bone and cartilage to stimulate matrix formation and remodeling of the wounded area. Bone tissue is a living tissue that is continuously being remodeled by the processes of resorption and deposition of bone matrix and minerals. This process typically involves osteoclasts and osteoblasts. Remodeling is initiated when osteoclasts or osteoblasts are recruited from, e.g., bone marrow or the circulation, to the bone. New bone formation is classified according to three basic processes: osteogenesis, osteoconduction and osteoinduction.

Cartilage is a specialized dense connective tissue consisting of cells in a matrix. There are several kinds of cartilage, including translucent cartilage, articular cartilage, costal cartilage, fibrous cartilage, and yellow cartilage. Cartilage is a tissue made of an extracellular matrix primarily comprised of the organic compounds collagen, hyaluronic acid, and chondrocyte cells, which are responsible for cartilage production. Collagen, hyaluronic acid and water entrapped within these organic matrix elements yield the unique elastic properties and strength of cartilage. In cartilage, collagen synthesis is typically required for growth and repair, as well as for the successful bonding of grafts and prosthetic devices. Collagen is the major structural protein responsible for the architectural integrity of cartilage.

Factors that stimulate or destimulate these mechanisms of angiogenesis, bone, or cartilage growth are expected to be effective for treatments that involve such mechanisms. Certain embodiments herein are directed towards using the factors set forth herein alone or in combination with other factors to treat conditions involving angiogenesis, bone, or cartilage growth or repair.

Nucleic Acids, Polypeptides, Identity, Hybridization, and Stringency

As used herein, the term nucleic acid refers to both RNA and DNA, including cDNA, genomic DNA, synthetic (e.g., chemically synthesized) DNA, as well as naturally-occurring and chemically modified nucleic acids, e.g., synthetic bases or alternative backbones. A nucleic acid molecule can be double-stranded or single-stranded (i.e., a sense or an antisense single strand).

An isolated nucleic acid refers to a nucleic acid that is separated from other nucleic acid bases that are present in a genome, including nucleic acids that normally flank one or both sides of a nucleic acid sequence in a vertebrate genome (e.g., nucleic acids that flank a gene). The term isolated as used herein with respect to nucleic acids also includes non-naturally-occurring nucleic acid sequences, since such non-naturally-occurring sequences are not found in nature and do not have immediately contiguous sequences in a naturally-occurring genome.

An isolated nucleic acid can be, for example, a DNA molecule, provided at least one of the nucleic acid sequences normally found flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid because such sources do not indicate a role for the nucleic acid or its uses. Indeed, there is often no knowledge of the sequences present in such sources until their presence is hypothesized as a result of using hindsight in light of a new sequence.

Isolated nucleic acid molecules can be produced by standard techniques, including, without limitation, common molecular cloning and chemical nucleic acid synthesis techniques, e.g., polymerase chain reaction (PCR), chemical synthesis either as a single nucleic acid molecule (e.g., using automated DNA synthesis in the 3′ to 5′ direction using phosphoramidite technology) or as a series of polynucleotides. For example, one or more pairs of long polynucleotides (e.g., >100 nucleotides) can be synthesized that contain the desired sequence, with each pair containing a short segment of complementarity (e.g., about 15 nucleotides) such that a duplex is formed when the polynucleotide pair is annealed. DNA polymerase may be used to extend the polynucleotides, resulting in a single, double-stranded nucleic acid molecule per polynucleotide pair.

“Polynucleotides” are nucleic acid molecules of at least three nucleotide subunits. A nucleotide has three components: an organic base (e.g., adenine, cytosine, guanine, thymine,, or uracil, herein referred to as A, C, G, T, and U, respectively), a phosphate group, and a five-carbon sugar that links the phosphate group and the organic base. In a polynucleotide, the organic bases of the nucleotide subunits determine the sequence of the polynucleotide and allow for interaction with a second polynucleotide. The nucleotide subunits of a polynucleotide are linked by phosphodiester bonds such that the five-carbon sugar of one nucleotide forms an ester bond with the phosphate of an adjacent nucleotide, and the resulting sugar-phosphates form the backbone of the polynucleotide.

Polynucleotide analogues or polynucleic acids are chemically modified polynucleotides or polynucleic acids. In some embodiments, polynucleotide analogues can be generated by replacing portions of the sugar-phosphate backbone of a polynucleotide with alternative functional groups. Morpholino-modified polynucleotides, referred to herein as “morpholinos,” are polynucleotide analogues in which the bases are linked by a morpholino-phosphorodiamidate backbone (See, Summerton and Weller (1997) Antisense Nuc. Acid Drug Devel. 7:187-195; and U.S. Pat. Nos. 5,142,047 and 5,185,444).

In addition to morpholinos, other examples of polynucleotide analogues include analogues in which the bases are linked by a polyvinyl backbone (Pitha et al. (1970) Biochim. Biophys. Acta 204:39-48; Pitha et al. (1970) Biopolymers 9:965-977), peptide nucleic acids (PNAs) in which the bases are linked by amide bonds formed by pseudopeptide 2-aminoethyl-glycine groups (Nielsen et al. (1991) Science 254:1497-1500), analogues in which the nucleoside subunits are linked by methylphosphonate groups (Miller et al. (1979) Biochem. 18:5134-5143; Miller et al. (1980) J. Biol. Chem. 255:9659-9665), analogues in which the phosphate residues linking nucleoside subunits are replaced by phosphoroamidate groups (Froehler et al. (1988) Nucleic Acids Res. 156:4831-4839), and phosphorothioated DNAs, analogues containing sugar moieties that have 2′ O-methyl groups (Cook (1998) Antisense Medicinal Chemistry, Springer, New York, pp. 51-101).

Polynucleotides of the invention can be produced through the well-known and routinely used technique of solid phase synthesis. Equipment for such synthesis is commercially available from several vendors including, for example, Applied Biosystems (Foster City, Calif.). Alternatively, other suitable methods for such synthesis can be used (e.g., common molecular cloning and chemical nucleic acid synthesis techniques). Similar techniques also can be used to prepare polynucleotide analogues such as morpholinos or phosphorothioate derivatives. In addition, polynucleotides and polynucleotide analogues can be obtained commercially from, for example, Gene Tools, L. L. C. (Philomath, Oreg.) or Oligos Etc. (Wilsonville, Oreg.).

Typically, polynucleotide analogues such as morpholinos are single stranded. Polynucleotide analogues can be of various lengths (e.g., from 8 bases in length to more than 112 bases in length, typically from 12 to 72 bases in length). Morpholinos can be, for example, 15 to 45 bases in length (e.g., 18 to 30 bases in length). Polynucleotide analogues can be designed to contain certain percentages of each base type (e.g., 40-60% A/T content and 40-60% G/C content, or 50% A/T content and 50% G/C content). In addition, it is sometimes useful to avoid sequences containing four or more consecutive G residues, as well as secondary structures such as hairpins.

Polynucleotides and polynucleotide analogues (e.g., morpholinos) can be designed to hybridize to a target nucleic acid molecule. The term hybridization, as used herein, means hydrogen bonding, which can be Watson-Crick, Hoogsteen, or reversed Hoogsteen hydrogen bonding, between complementary nucleoside or nucleotide bases. For example, A and T, and G and C, respectively, are complementary bases that pair through the formation of hydrogen bonds. Complementary, as used herein, refers to the capacity for precise pairing between two nucleotides. A nonspecific adsorption or interaction is not considered to be hybridization. For example, if a nucleotide at a certain position of a polynucleotide analogue is capable of hydrogen bonding with a nucleotide at the same position of a target nucleic acid molecule, then the polynucleotide analogue and the target nucleic acid molecule are considered to be complementary to each other at that position. A polynucleotide or polynucleotide analogue and a target nucleic acid molecule are complementary to each other when a sufficient number of corresponding positions in each molecule are occupied by nucleotides that can hydrogen bond with each other. It is understood in the art that the sequence of the polynucleotide or polynucleotide analogue need not be 100% complementary to that of the target nucleic acid molecule to hybridize.

Hybridization conditions in vitro of nucleic acids and nucleic acid analogs are dependent upon temperature, nucleic acid probe length, salt concentration, solvent concentration, and the G+C content of the probe (Sambrook, J., 1989). Typically, conditions of high to moderate stringency are used for specific hybridization in vitro, such that hybridization occurs between substantially similar nucleic acid, but not between dissimilar nucleic acids. High stringency hybridization is carried out under the following conditions for DNA probes (100 to 1000 base pairs) that hybridize to DNA or RNA: 50% formamide, 5×SSC (0.75 M Sodium Chloride/0.075 Sodium Citrate), 0.1% Sodium Dodecyl Sulfate (SDS) at 42° C. for 12 hours. This is followed by washing 4 times in 0.2×SSC/0.1% SDS for 30 minutes each at 42° C.

The melting temperature (Tm) of the hybrid between a probe and its target can be calculated by the following equation (Bolton, E. T. 1962): Tm=81.5° C.+16.6(log₁₀[Na+])+0.41(fraction G+C)−0.63(%formamide)−(600/l) where l is the length of the hybrid in base pairs. In general high stringency hybridizations are carried out between 20 to 25° C. below the Tm, and washing conditions are carried out between 12 to 20° C. below the Tm.

For probes smaller than 100 bp, the following equation can be used (Bolton, E. T. 1962): Tm=81.5° C.+16.6(log₁₀[Na+])+0.41(fraction G+C)−(600/N) where N is the chain length. For the smaller probes, high stringency hybridizations are carried out between 5 to 10° C. below the Tm, and washing conditions are quickly carried out between 5 to 10° C. below the Tm.

In vivo hybridization conditions consist of intracellular conditions (e.g., physiological pH and intracellular ionic conditions) that govern the hybridization of polynucleotides and polynucleotide analogues with target nucleic acid molecules. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. Alternatively, a wash solution containing 4×SSC, 0.1% SDS can be used at 37° C., with a final wash in 1×SSC at 45° C. In order for a polynucleotide or polynucleotide analogue to specifically decrease expression from a target nucleic acid molecule, the polynucleotide or polynucleotide analogue hybridizes to the target nucleic acid molecule under physiological conditions.

Polynucleic acids and polynucleic acid analogue embodiments can be useful for research and diagnostics, and for therapeutic use. For example, assays based on hybridization of polynucleotide analogues to nucleic acids encoding PTV, or PTV fragments, can be used to evaluate levels of the polypeptide in a tissue sample. Hybridization of a polynucleotide analogue of the invention with a target nucleic acid molecule can be detected by a number of methods. Some of these methods are well known in the art, and including detection by conjugating an enzyme to the polynucleotide analogues or by radiolabeling of the polynucleotide analogues. Any other suitable means of detection also can be used. Additionally, polynucleotides and polynucleotide analogues can be employed as therapeutic moieties in the treatment of disease states in animals, including humans.

Certain embodiments provide various polypeptide sequences and/or purified polypeptides. A polypeptide refers to a chain of amino acid residues, regardless of post-translational modification (e.g., phosphorylation or glycosylation) and/or complexation with additional polypeptides, synthesis into multisubunit complexes, with nucleic acids and/or carbohydrates, or other molecules. Proteoglycans therefore also are referred to herein as polypeptides. As used herein, a “functional polypeptide” is a polypeptide that is capable of promoting the indicated function. Polypeptides can be produced by a number of methods, many of which are well known in the art. By way of example and not limitation, polypeptides can be obtained by extraction from a natural source (e.g., from isolated cells, tissues or bodily fluids), by expression of a recombinant nucleic acid encoding the polypeptide, or by chemical synthesis. Polypeptides can be produced by, for example, recombinant technology, and expression vectors encoding the polypeptide introduced into host cells (e.g., by transformation or transfection) for expression of the encoded polypeptide.

Expression systems that can be used for small or large scale production of polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

The term purified as used herein with reference to a polypeptide refers to a polypeptide that either has no naturally occurring counterpart (e.g., a peptidomimetic), or has been chemically synthesized and is thus substantially uncontaminated by other polypeptides, or has been separated or purified from other most cellular components by which it is naturally accompanied (e.g., other cellular proteins, polynucleotides, or cellular components). An example of a purified polypeptide is one that is at least 70%, by dry weight, free from the proteins and naturally occurring organic molecules with which it naturally associates. A preparation of the a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag, or a Flag® tag) that facilitates the polypeptide to be purified or marked (e.g., captured onto an affinity matrix, visualized under a microscope).

The identity of a protein or nucleic acid sequence is frequently established based on a sequence alignment of the DNA, RNA, or amino acids. Multiple alignments of such sequences are important tools in studying biomolecules. The basic information they provide is identification of conserved sequence regions. This is very useful in designing experiments to test and modify the function of specific proteins, in predicting the function and structure of proteins, and in identifying new members of protein families. Sequences can be aligned across their entire length (global alignment) or only in certain regions (local alignment). This is true for pairwise and multiple alignments. Global alignments with respect to polynucleic acids or polypeptides usually need to use gaps (representing insertions/deletions) while local alignments can usually avoid them by aligning regions between gaps. In a sequence alignment, letters arranged over one another are called matched. If two matched letters are equal, the match is called an identity otherwise the match is called a substitution or mismatch. An insertion or deletion (indel) is one or more letters aligned against a gap (−) and is considered the same as a mismatch for percent identity purposes (Waterman, M. S. 1995).

In some cases a determination of the percent identity of a peptide to a sequence set forth herein may be required. In such cases, the percent identity is measured in terms of the number of residues of the peptide, or a portion of the peptide. Thus a peptide of 10 residues would be 90% identical to SEQ ID NO 18 if nine of the residues of the peptide were determined to be matched to SEQ ID NO 18. A peptide or polypeptide of, e.g., 90% identity, may also be a portion of a larger peptide; for example, a peptide of 100 residues that has a portion that is 10 residues in length that is matched to 9 residues of SEQ ID NO 36 would have 90% identity with SEQ ID NO 36.

The amino acid residues described herein employ either the single letter amino acid designator or the three-letter abbreviation. Abbreviations used herein are in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

Although particular amino acid sequences have been described herein, there are a variety of conservative changes that can be made to an amino acid sequence without altering activity. These changes are termed conservative mutations, that is, an amino acid belonging to a grouping of amino acids having a particular size or characteristic can be substituted for another amino acid. Substitutes for an amino acid sequence may be selected from other members of the class to which the amino acid belongs. For example, the nonpolar (hydrophobic) amino acids include alanine, leucine, isoleucine, valine, proline, phenylalanine, tryptophan, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such alterations are not expected to substantially affect apparent molecular weight as determined by polyacrylamide gel electrophoresis or isoelectric point. Particularly preferred conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free NH₂. Moreover, point mutations, deletions, and insertions of the polypeptide sequences or corresponding nucleic acid sequences may in some cases be made without a loss of function of the polypeptide or nucleic acid fragment.

There are other DNA or RNA sequences encoding TDE1, PTV, MOESIN, and HKE4 proteins that have the same amino acid sequence as an TDE1, PTV, MOESIN, and HKE4 protein, but which take advantage of the degeneracy of the three letter codons used to specify a particular amino acid.

Antisense Molecules

A polynucleic acid or polynucleic acid analogue can be complementary to a sense or an antisense target nucleic acid molecule. When complementary to a sense nucleic acid molecule, the polynucleic acid is said to be antisense. When complementary to an antisense nucleic acid molecule, the polynucleotide analogue is said to be sense. For example, a polynucleotide analogue can be antisense to an mRNA molecule or sense to the DNA molecule from which an mRNA is transcribed. As used herein, the term “coding region” refers to the portion of a nucleic acid molecule encoding an RNA molecule that is translated into protein. A polynucleotide or polynucleotide analogue can be complementary to the coding region of an mRNA molecule or the region corresponding to the coding region on the antisense DNA strand. Alternatively, a polynucleotide or polynucleotide analogue can be complementary to the non-coding region of a nucleic acid molecule. A non-coding region can be, for example, upstream of a transcriptional start site or downstream of a transcriptional end-point in a DNA molecule. A non-coding region also can be upstream of the translational start codon or downstream of the stop codon in an mRNA molecule. Furthermore, a polynucleotide or polynucleotide analogue can be complementary to both coding and non-coding regions of a target nucleic acid molecule. For example, a polynucleotide analogue can be complementary to a region that includes a portion of the 5′ untranslated region (5′-UTR) leading up to the start codon, the start codon, and coding sequences immediately following the start codon of a target nucleic acid molecule.

Various antisense molecules are set forth herein. In some embodiments, the antisense molecules can be preferably targeted to hybridize to the start codon of a mRNA and to codons on either side of the start codon, e.g., within 1-20 bases of the start codon. Other codons, however, may be targeted with success, e.g., any set of codons in a sequence. The procedure for identifying additional antisense molecules will be apparent to an artisan of ordinary skill after reading this disclosure. One procedure would be to test antisense molecules of about 20 nucleic acids in a high-throughput screening assay such as zebrafish embryos or cultured cell line. Each proposed antisense molecule would be tested to determine its effectiveness, and the most promising candidates would form the basis for optimization.

Hybridization of antisense oligonucleotides with mRNA interferes with one or more of the normal functions of mRNA, e.g., translocation of the RNA to a site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Polynucleic acids, such as the sequences set forth herein and fragments thereof, can be used in diagnostics, therapeutics, prophylaxis, and as research reagents and in kits. Provision of means for detecting hybridization of oligonucleotide with a gene, mRNA, or polypeptide can routinely be accomplished. Such provision may include enzyme conjugation, radiolabelling or any other suitable detection systems. Research purposes are also available, e.g., specific hybridization exhibited by the polynucleotides or polynucleic acids may be used for assays, purifications, cellular product preparations and in other methodologies which may be appreciated by persons of ordinary skill in the art.

Modified nucleic acids are known and may be used with embodiments described herein, for example as described in Antisense Research and Application (Springer-Verlag, Berlin, 1998), and especially as described in the chapter by S. T. Crooke: Chapter 1: Basic Principles of Antisense Therapeutics pp. 1-50; and in Chapter 2 by P. D. Cook: Antisense Medicinal Chemistry pp. 51-101. Some modified backbones for nucleic acid molecules are, for example, morpholinos, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Another modification of the oligonucleotides set forth herein involves chemically linking to the oligonucleotide one or more moieties or conjugates which enhance the activity, cellular distribution or cellular uptake of the oligonucleotide. Other embodiments include chimeric oligonucleotides. “Chimeric” oligonucleotides or “chimeras,” in the context of this invention, are oligonucleotides that contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. Examples of chimeric oligonucleotides include but are not limited to “gapmers,” in which three distinct regions are present, normally with a central region flanked by two regions that are chemically equivalent to each other but distinct from the gap. Other chimeras include “wingmers,” also known in the art as “hemimers,” that is, oligonucleotides with two distinct regions.

For oligonucleotides, examples of pharmaceutically acceptable salts include but are not limited to (a) salts formed with cations such as sodium, potassium, ammonium, etc.; (b) acid addition salts formed with inorganic acids, for example, hydrochloric acid, hydrobromic acid (c) salts formed with organic acids e.g., for example, acetic acid, oxalic acid, tartaric acid; and (d) salts formed from elemental anions e.g., chlorine, bromine, and iodine.

In general, for any substance, a pharmaceutically acceptable carrier is a material that is combined with the substance for delivery to an animal. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable carriers are used, in general, with a compound so as to make the compound useful for a therapy or as a product.

Vectors

Nucleic acids can be incorporated into vectors. As used herein, a vector is a replicon, such as a plasmid, phage, or cosmid, into which another nucleic acid segment may be inserted so as to bring about replication of the inserted segment. Vectors of the invention typically are expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, “operably linked” means that the expression control sequence and the inserted nucleic acid sequence of interest are positioned such that the inserted sequence is transcribed (e.g., when the vector is introduced into a host cell).. For example, a DNA sequence is operably linked to an expression-control sequence, such as a promoter when the expression control sequence controls and regulates the transcription and translation of that DNA sequence. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. Examples of vectors include: plasmids, adenovirus, Adeno-Associated Virus (AAV), Lentivirus (FIV), Retrovirus (MoMLV), and transposons.

There are a variety of promoters that could be used including, but not limited to, constitutive promoters, tissue-specific promoters, inducible promoters, and the like. Promoters are regulatory signals that bind RNA polymerase in a cell to initiate transcription of a downstream (3′ direction) coding sequence.

A particularly useful vector is a tranposase/transposon system for introducing nucleic acid sequences into the DNA of a cell, as set forth in U.S. Pat. No. 6,489,458 and U.S. patent Ser. Nos. 09/191,572 entitled “Nucleic Acid Transfer Vector For The Introduction Of Nucleic Acid Into The DNA Of A Cell”; Ser. No. 09/569,257 entitled “Vector-Mediated Delivery Of Integrating Transposon Sequences”; Ser. No. 10/128,998 entitled “Transposon System For Gene Delivery In Vertebrates”; and Ser. No. 10/128,998 “Composition For Delivery Of Compounds To Cells”. A transposase is an enzyme that is capable of binding to DNA at regions of DNA termed inverted repeats. Transposons typically contain at least one, and preferably two, inverted repeats that flank an intervening nucleic acid sequence. The transposase binds to recognition sites in the inverted repeats and catalyzes the incorporation of the transposon into DNA. Transposons can be mobile, in that they can move from one position on DNA to a second position on DNA in the presence of a transposase. There are typically two components of a mobile cut-and-paste type transposon system, a source of an active transposase, and the DNA sequences that are recognized and mobilized by the transposase. Mobilization of the DNA sequences permits the intervening nucleic acid between the recognized DNA sequences to also be mobilized.

Examples of Uses for tde1, ptv, Moesin, and hke4

Examples of uses for tde1, ptv, moesin, and hke4 include the generation of protein products, inhibition of expression of the proteins or mRNAs, administration of materials and products, screening procedures, and techniques for making drugs, as well as therapeutics, vectors, probes, and as a source of epitopes for creating antibodies. The capitalized names of polypeptides are sometimes used for convenience but the embodiments are meant to also include RNA or DNA as an alternative embodiment.

Fragments of TDE1, PTV, MOESIN, and HKE4 RNA or DNA are particularly useful for making probes for their family members so that further aspects of their role may be documented. For example, tde, ptv, or moesin fragments may be used to make hybridization probes to show levels of tde, ptv, or moesin expression in cell cultures and thereby deduce blood vessel formation activity. For example, hke4 fragments may be used to make hybridization probes to show levels of hke4 expression in cell cultures and thereby deduce bone or cartilage formation activity. It is to be understood that blood vessel formation activity encompasses both the formation of blood vessels and processes that interfere with blood vessel formation, and also changes to blood vessel morphology, function, structure, or activity. Further, fragments of the polypeptides TDE1, PTV, MOESIN, and HKE4 are useful as probes for nucleic acid aptamers, and as probes to elucidate the active domains of the polypeptide so that the function of TDE1, PTV, MOESIN, and HKE4 may be modulated.

Similarly, fragments of complements of nucleic acid sequences for TDE1, PTV, MOESIN, and HKE4 are useful for making antisense molecules to inhibit the expression of TDE1, PTV, MOESIN, or HKE4 expression. Embodiments include: DNA, RNA, or polypeptide fragments of TDE1, PTV, MOESIN, and HKE4 of at least 3, 5, 6, 8, 10, 12, or 14 residues, and includes fragments ranging from 3 to 1000 residues, as well as any and all ranges in between 3 and 1000. Some convenient fragment sizes are in the range of 6 to 25 residues, from 8 to 16 residues, and from 10 or 12 residues to 20 or 25 residues. A choice of fragment sizes depends on, e.g., the degree of specificity that is desirable, cost of manufacturing, desired binding constant, and the particular application.

In general, TDE1, PTV, and MOESIN are useful for conditions wherein blood vessel formation is to be promoted, for example in wound healing, or inhibited, for example, as with cancer tumor treatments. For example, a TDE 1, PTV, or MOESIN derived compound, e.g., a polypeptide, nucleic acid, fragment thereof, a mimic, imitator, agonist, or an antagonist may be applied. Embodiments include dominant negative fragments of the polypeptides. Application may be made, for example, locally, systemically, or at or near the desired site of effect. At or near means within less than about 3 cm, while locally means within less than about 10 cm, and systemically means throughout all or most of the body.

Examples of wound healing include application to a site of necrotic tissue, an ischemic tissue, or an injured tissue. For example, an ulcer having some necrotic tissue may receive a dose of TDE 1, PTV, and/or MOESIN that is effective to help promote blood vessel formation. Necrotic tissue is a pathology that may appear in essentially any tissue, including the limbs, the heart, epidermis, dermis, and muscle. Of particular concern are diabetic ulcers and compression ulcers, as well as burned tissues. For example, a TDE1, PTV, or MOESIN derived compound may be injected into a wound site or into tissue near it. In the case of a diabetic ulcer, for example, the compound may be introduced topically by, for example, applying it in a carrier to the wound and the area around the wound. Controlled release carriers or matrices may also be used, e.g., hydrogels, pastes, gels, crosslinked polymers, and control release capsules. An example of an ischemic tissue is a heart that receives a suboptimal amount of blood because of injury or disease. A TDE1, PTV, or MOESIN derived compound may be introduced into the heart or near the heart, for example, in or near the coronary arteries. Introduction may be, e.g., by injection or percutaneous minimally invasive surgical procedure. Examples of injured tissues include burns, puncture, and debridement.

Examples of medical aspects of inhibiting blood vessel formation include, for example, treating tumors to reduce blood flow, control of unwanted capillary invasion, and undesired blood vessel formation associated with a medical device. Some pathologies of the visual system entail unwanted blood vessel formation; such formation may be inhibited using a TDE1, PTV, or MOESIN derived compound, e.g., by introducing it into or near the eye. Restenosis is a condition associated with blood vessel narrowing in the vicinity of a medical stent. One aspect of blood vessel formation is the control of endothelial cells and associated cells. A TDE1, PTV, and/or MOESIN derived compound may be introduced in conjunction with a stent to prevent unwanted vessel formation and/or to control endothelial cell activity.

Certain embodiments also provide antibodies having specific binding activity for a polypeptide (e.g., polypeptides of TDE1, PTV, MOESIN, HKE4, or complexes or fragments thereof). Such antibodies can be useful for detecting levels of the polypeptide in cells treated with morpholinos, for example. Antibodies also can be useful as polypeptide-modulating agents, e.g., to affect their activity and hereby increase or decrease it. A polypeptide as described herein can act as an immunogen to elicit an antibody response that is specific to the polypeptide or larger protein, for example, and does not cross-react with a different polypeptide. A specific antibody directed to a fragment of a TDE1, PTV, MOESIN, or HKE4 polypeptide therefore will specifically recognize that polypeptide, without substantial binding or hybridizing to other polypeptides that may be present in the same biological sample.

The term antibody or antibodies includes intact molecules as well as fragments thereof that are capable of binding to an epitope of a polypeptide, e.g., TDE1, PTV, MOESIN, or HKE4. The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. Other sizes of polypeptides may be used, however, to generate larger number of antibodies and to affect antigenicity, including peptides of about 8, about 10, about 12, about 15, or within the range of from about 15 to about 30 residues. The terms antibody and antibodies include polyclonal antibodies, monoclonal antibodies, humanized or chimeric antibodies, single chain Fv antibody fragments, Fab fragments, and F(ab)₂ fragments.

In general, can be polypeptide is produced as described above, e.g., recombinantly, by chemical synthesis, or by purification of the native protein, and then used to immunize animals. Various host animals including, for example, horses, pigs, sheep, goats, rabbits, chickens, mice, guinea pigs, and rats, can be immunized by injection of the protein of interest. Depending on the host species, adjuvants can be used to increase the immunological response. These include Freund's adjuvant (complete and/or incomplete), mineral gels such as aluminum hydroxide, surface-active substances such as lysolecithin, pluronic polyols, polyanions, peptides, oil emulsions, keyhole limpet hemocyanin, and dinitrophenol. Polyclonal antibodies are contained in the sera of the immunized animals. Monoclonal antibodies can be prepared using standard hybridoma technology. In particular, monoclonal antibodies can be obtained by any technique that provides for the production of antibody molecules by, for example, continuous cell lines in culture as described by Kohler et al. [(1975) Nature 256:495-497]; the human B-cell hybridoma technique of Kosbor et al. [(1983) Immunology Today 4:72] and Cote et al. [(1983) Proc. Natl. Acad. Sci. USA 80:2026-2030]; and the EBV-hybridoma technique of Cole et al. [Monoclonal Antibodies and Cancer Therapy, Alan R. Liss, Inc. pp. 77-96 (1983)]. Such antibodies can be of any immunoglobulin class, including IgM, IgG, IgE, IgA, IgD, and any subclass thereof. A hybridoma producing the monoclonal antibodies of the invention can be cultivated in vitro or in vivo. A chimeric antibody can be a molecule in which different portions are derived from different animal species, such as those having a variable region derived from a mouse monoclonal antibody and a human immunoglobulin constant region. Chimeric antibodies can be produced through standard techniques.

A monoclonal antibody also can be obtained by using commercially available kits that aid in preparing and screening antibody phage display libraries. An antibody phage display library is a library of recombinant combinatorial immunoglobulin molecules. Examples of kits that can be used to prepare and screen antibody phage display libraries include the Recombinant Phage Antibody System (Pharmacia, Peapack, N.J.) and SurfZAP Phage Display Kit (Stratagene, La Jolla, Calif.). Once produced, antibodies or fragments thereof can be tested for recognition of a polypeptide by standard immunoassay methods including, for example, enzyme-linked immunosorbent assay (ELISA) or radioimmuno assay (RIA). See, Short Protocols in Molecular Biology, eds. Ausubel et al., Green Publishing Associates and John Wiley & Sons (1992). Antibodies that have equal binding affinities for recombinant and native proteins are particularly useful.

The present invention is also suitable for diagnosing abnormal proliferative states in tissue or other samples from patients suspected of having a hyperproliferative disease such as cancer. The ability of the oligonucleotides of the present invention to inhibit cell proliferation may be employed to diagnose such states. A number of assays may be formulated employing the present invention, which assays will commonly comprise contacting a tissue sample with an oligonucleotide of the invention under conditions selected to permit detection and, usually, quantitation of such inhibition. In the context of this invention, to “contact” tissues or cells with an oligonucleotide or oligonucleotides means to add the oligonucleotide(s), usually in a liquid carrier, to a cell suspension or tissue sample, either in vitro or ex vivo, or to administer the oligonucleotide(s) to cells or tissues within an animal. Thus TDE1, PTV, and MOESIN, or fragments thereof, can be used to diagnose or visualize cancer.

HKE4 is useful as a probe for bone and cartilage formation. Thus HKE4, hke4, or fragments may be use to monitor changes in bone or cartilage formation in response to other treatments. Moreover, HKE4 or its inhibitors may be used in conditions wherein bone or cartilage is deficient or grows pathologically. Examples of such conditions include, for example, osteoporosis, myositis ossificans, arachnoiditis ossificans, fibrodysplasia ossificans, and tracheobronchopathia osteochondroplastica. Moreover, HKE4 may be used to augment maxillofacial or reconstructive cartilage and/or bone procedures. For example, it may be added to a delivery vehicle and left in the patient. Examples of delivery vehicles include, for example, saline, pastes, tissue glues, fibrin glues, and synthetic matrices.

Screening and Production Assays

Certain embodiments involve using TDE1, PTV, MOESIN, or HKE4 proteins or nucleic acids, fragments thereof, mutants thereof, or other derivatives thereof in screening assays. Such assays are useful for identifying compounds that interact with TDE1, PTV, MOESIN, or HKE4. Such compounds may be used, for example, as markers for TDE1, PTV, MOESIN, or HKE4 or as modulators of their activity. The markers may be used to study, for example, angiogenesis, vasculogenesis, blood vessel formation, or bone and cartilage formation, as would be clear from the disclosures herein relating the various aspects of TDE1, PTV, MOESIN, or HKE4.

An embodiment involves the use, for example, of TDE1, PTV, MOESIN, or HKE4 to isolate a factor that is interactive thereto. In general, a suitable technique may involve: (a) generating a set of factors (sometimes referred to as a library) based on intelligent design, random generation, or a combination thereof and (b) screening the set of factors to determine which factors become associated with the target. A preferred association is by specific binding, which is a binding to the target with a much greater affinity than to non-target molecules. Some embodiments involve use of TDE1, PTV, MOESIN, or HKE4 proteins or nucleic acids, fragments thereof, mutants thereof, or other derivatives thereof as targets. For example, a polypeptide can be bound to a solid-phase surface and a solution comprising one or more factors is exposed to the bound polypeptide. The surface is subsequently separated from the solution to isolate the factors that are bound to the polypeptide. Then, the factors that bind to the polypeptide can be identified. Isolation can involve other techniques besides solid phase binding, including, for example, flocculation, aggregation, precipitation, use of magnetic markers, and fluorescent markers.

Some embodiments for screening involve techniques that are sometimes referred to as combinatorial chemistries. There are various techniques for performing combinatorial library production that are applicable to the production of factors see, e.g., U.S. Pat. Nos. 5,424,186; 5,449,754; 5,503,805; 5,650,489; 5,962,736; 6,042,789; 6,051,439; 6,083,682; 6,117,397; 6,168,913; 6,168,914; and 6,355,490. Such techniques may include use of biological libraries, spatially addressable parallel or solid phase solution libraries, synthetic library methods requiring deconvolution, a one-bead, one-compound method, and synthetic library methods using affinity chromatography selection. The library is then screened to determine which factors become associated with a target. A library is a set of molecules that are tested. Additional combinatorial approaches to screening include, for example, those set forth in U.S. Pat. Nos. 6,429,025; 6,432,651; and 5,783,397.

Combinatorial production methods for the libraries have various embodiments. Spatially addressable parallel solid phase or solution-phase libraries include, for example, multi-pin technology, SPOTs-membrane, light-directed peptide synthesis on chips, and diversomer technology. Synthetic libraries requiring deconvolution include an iterative approach, positional scanning, recursive deconvolution, and orthogonal partition approaches. Split-pool and iterative deconvolution combinatorial synthesis approaches are also available; however, other techniques may also be applied, including positional scanning, array synthesis, non-linear, double, and orthogonal strategies.

A multiplicity of screening strategies is available. One approach is the use of a solid phase assay. The factors are attached to a solid support, e.g., a chip, pin, bead, plastic sheet, glass, filamentous phage. The target is added to the support and the factors are examined for biological activity. Such activity may include for example, binding, or a functional assay such as proteolysis or phosphorylation. Binding can be conveniently measured directly, (e.g., by visualization of a dye on the target) or indirectly (e.g., by a reporter groups such as an enzyme).

Another screening method involves solution-phase assays. The factors are in a solution that is exposed to the target. The interaction between the factors and the target is detected and the factor is isolated. Examples of such techniques include competitive receptor binding assays with a known radiolabeled target or factor, competitive ELISA assay using plate-coated antigens, enzymatic assays such as proteolytic assay using a fluorgenic substrate, anti-bacterial assays, and cell-based signal transduction assays.

To identify TDE1, PTV, MOESIN, or HKE4-modulating agents, a cell that produces TDE1, PTV, MOESIN, or HKE4 polypeptides can be contacted with a candidate agent (e.g., a morpholino designed to hybridize to a target nucleic acid molecule encoding TDE1, PTV, MOESIN, or HKE4), and the amount of the tde1, ptv, moesin, or hke4 polypeptide or mRNA encoding the TDE1, PTV, MOESIN, or HKE4 polypeptide can be determined.

Examples of TDE 1, PTV, MOESIN, or HKE4-modulating agents that decrease levels of TDE1, PTV, MOESIN, or HKE4 polypeptides include morpholinos, antisense molecules, and antibodies against TDE1, PTV, MOESIN, or HKE4. Examples of TDE1, PTV, MOESIN, or HKE4-modulating agents that increase levels of TDE1, PTV, MOESIN, or HKE4 polypeptides include TDE1, PTV, MOESIN, or HKE4 polypeptides and nucleic acids encoding TDE1, PTV, MOESIN, or HKE4 polypeptides.

Other screening embodiments include use of cells to show how factors interact with biomolecules in vitro and to predict their action in vivo. For example, a factor that binds to a TDE1, PTV, MOESIN, or HKE4 nucleic acid sequence may be introduced into a cell using standard techniques. The cell may then to be tested to determine how the expression of TDE1, PTV, MOESIN, or HKE4 is affected by the factor. One method of testing the cell is to measure the TDE1, PTV, MOESIN, or HKE4 polypeptide levels for cells that have been treated with a factor that associates with TDE1, PTV, MOESIN, or HKE4 and to compare them to control cells that have not received the factor. If the treated cells have reduced TDE1, PTV, MOESTN, or HKE4 polypeptide expression, then the factor is an inhibitor of TDE1, PTV, MOESIN, or HKE4 inhibition. Many cell lines are known, including human cells and cells from vertebrates, invertebrates, and bacteria, and the choice of a suitable model usually depends on the factor and its target. Further, a compound for administration to an animal may be made by screening being TDE1, PTV, MOESIN, or HKE4 proteins or nucleic acids, fragments thereof, mutants thereof, or other derivatives thereof for a specifically binding factor, and making that factor using standard synthesis techniques.

Methods of making a drug are also set forth herein. These methods involve using standardized techniques for making factors that bind to a target, with the target being TDE1, PTV, MOESIN, or HKE4 proteins or nucleic acids, fragments thereof, mutants thereof, or other derivatives thereof. The factors that bind the target may be made to inhibit the action of the target. Inhibition can be, for example, by steric hindrance, antisense binding, and other methods known to those skilled in these arts. The factors may then be combined with a pharmaceutically acceptable carrier and administered to a cell, e.g., in an animal, to inhibit the target in the animal.

A compound for administration to an animal may be made by screening TDE1, PTV, MOESIN, or HKE4 proteins or nucleic acids, fragments thereof, mutants thereof, or other derivatives thereof for a specifically binding factor and making that factor using standard manufacturing techniques.

Therapeutics and Pharmacologics

Set forth herein are methods for identifying substances that specifically increase or decrease the amount of a TDE1, PTV, MOESIN, or HKE4 polypeptide in a cell, tissue, organ, or organism of interest. A substance that specifically increases or decreases the amount of a TDE1, PTV, MOESIN, or HKE4 polypeptide may be herein referred to as a “modulating agent.” The amount of a TDE1, PTV, MOESIN, or HKE4 polypeptide in a cell can be assessed by, for example, conventional antibody-based assays. Alternatively, the amount of a TDE1, PTV, MOESIN, or HKE4 polypeptide can be estimated by detecting RNA using conventional nucleic acid-based assays [e.g., northern blotting or reverse transcription-polymerase chain reaction (RT-PCR)]. The amount of a TDE1, PTV, MOESIN, or HKE4 polypeptide in a cell can be modulated by increasing or decreasing the production of TDE1, PTV, MOESIN, or HKE4 mRNA and/or the amount of functional TDE1, PTV, MOESIN, or HKE4 polypeptide.

Polynucleotide analogues of the invention can be used to alter expression from a target tde1, ptv, moesin, or hke4 nucleic acid and thus can be modulating agents. As used herein, the term expression with respect to a nucleic acid molecule refers to production of an mRNA molecule from a DNA molecule and/or production of a polypeptide from an mRNA molecule. Expression from a nucleic acid molecule can be decreased, for example, by interfering with (1) any process necessary for mRNA transcription (e.g., binding of RNA polymerase, binding of transcription factors, or transcriptional elongation of the mRNA); (2) mRNA processing (e.g., capping or splicing); (3) mRNA transport across the nuclear membrane; or (4) any process necessary for mRNA translation (e.g., ribosome binding or translational initiation, elongation, or termination). Expression also can be decreased by inducing a cellular nuclease system that degrades cognate mRNAs. In an RNaseH dependent mechanism, for example, a double stranded target mRNA/DNA or RNA/polynucleotide analogue is degraded by RNaseH. In addition to polynucleotide analogues, conventional polynucleotides, such as antisense sequences, can be used to alter expression from target nucleic acid molecules to which they are complementary.

As used herein, a decrease with respect to expression from a target nucleic acid molecule refers to a decrease that can be detected by assessing changes in mRNA or protein levels. For example, a decrease can refer to a 5%, 10%, 25%, 50%, 75%, or more than a 75% decrease in expression. A decrease in expression also includes complete inhibition of expression, whereby a 100% decrease in expression from a nucleic acid molecule is achieved. Changes in mRNA and protein levels can be detected and/or measured by any of a number of methods known in the art, including but not limited to northern blotting or RT-PCR for mRNA assessment, and western blotting or enzyme-linked immunosorbent assays (ELISA) for protein assessment. Other suitable methods also can be used to assess mRNA and protein levels.

A decrease in expression from a target tde1, ptv, moesin, or hke4 nucleic acid molecule can be achieved using one polynucleotide analogue. A decrease in expression from a target nucleic acid molecule also can be achieved using two polynucleotide analogues having different sequences and therefore being complementary to different portions of the same target nucleic acid molecule. Similarly, decreases can be obtained with three or more polynucleotide analogues, or one or more conventional polynucleotides. A single polynucleotide analogue can be used to simultaneously decrease expression from two or more nucleic acid molecules that are closely related. In addition, multiple polynucleotide analogues having sequences complementary to more than one target nucleic acid molecule can be used to decrease expression from multiple target nucleic acid molecules at the same time.

Polynucleotide analogues such as morpholinos can be delivered to a living cell, tissue, organ, or organism of interest by methods used to deliver single stranded mRNA such as the methods described previously (Hyatt, T. M. 1999, Hackett and Alvarez, 2000). Non-limiting examples of delivery methods include (1) microinjection and (2) simply exposing the cell, tissue, organ, or organism of interest to the polynucleotide analogue. A cell can be, for example, a fertilized or unfertilized egg, or a cell in culture. A tissue can be any tissue regardless of its state of differentiation, and can include, for example, tumor tissue or normal tissue from an organism such as a mammal or a fish. An organ can be, for example, thymus, cartilage, bone marrow, pancreas, heart, or the blood vessels of the vasculature. Non-limiting examples of organisms include vertebrate embryos such as teleost embryos, juvenile animals, or adult animals. Examples of teleost embryos include zebrafish embryos, pufferfish embryos, medaka embryos, and stickleback embryos.

Polynucleic acid analogues can be delivered in a suitable buffer. A suitable buffer is one in which the polynucleotide analogue can be dissolved, and which is non-toxic to the cell, tissue, organ, or organism to which the polynucleotide analogue is to be delivered. A non-toxic buffer can be one that is isotonic to the organism or cell of interest. For example, morpholinos can be dissolved in Danieau buffer (see Example 4, below) for injection into zebrafish eggs or embryos.

Alternatively, a polynucleotide designed to hybridize to a target tde1, ptv, moesin, or hke4 nucleic acid molecule can be inserted into an expression vector that is then introduced into the cell, tissue, or organism of interest. For example, a polynucleotide in an expression vector can be operably linked to an expression control sequence, which will direct the production of a polynucleotide transcript that is capable of hybridizing to a target nucleic acid molecule. Methods for introducing a vector into a cell or an organism are known in the art (e.g., transformation, transfection, and microinjection).

Administration

The nucleic acids, polypeptides, antibodies, binding agents, and other compositions described herein relating to TDE1, PTV, MOESIN, HKE4, and VEGF may collectively be referred to as therapeutic agents. And such compositions and agents as set forth herein may be delivered by suitable means adapted to the application. Examples of delivery include via injection, including intravenously, intramuscularly, or subcutaneously, and in a pharmaceutically acceptable carriers, e.g., in solution and sterile vehicles, such as physiological buffers (e.g., saline solution or glucose serum). The embodiments may also be administered orally or rectally, when they are combined with pharmaceutically acceptable solid or liquid excipients. Embodiments can also be administered externally, for example, in the form of an aerosol with a suitable vehicle suitable for this mode of administration, for example, nasally. Further, delivery through a catheter or other surgical tubing is possible. Alternative routes include tablets, capsules, and the like, nebulizers for liquid formulations, and inhalers for lyophilized or acrosolized agents.

Presently known methods for delivering molecules in vivo and in vitro, especially small molecules, nucleic acids or polypeptides, may be used for the embodiments. Such methods include microspheres, liposomes, other microparticle vehicles or controlled release formulations placed in certain tissues, including blood. Examples of controlled release carriers include semipermeable polymer matrices in the form of shaped articles, e.g., suppositories, or microcapsules and U.S. Pat. Nos. 5,626,877; 5,891,108; 5,972,027; 6,041,252; 6,071,305, 6,074,673; 6,083,996; 6,086,582; 6,086,912; 6,110,498; 6,126,919; 6,132,765; 6,136,295; 6,142,939; 6,235,312; 6,235,313; 6,245,349; 6,251,079; 6,283,947; 6,283,949; 6,287,792; 6,296,621; 6,309,370; 6,309,375; 6,309,380; 6,309,410; 6,317,629; 6,346,272; 6,350,780; 6,379,382; 6,387,124; 6,387,397 and 6,296,832. Moreover, formulations for administration can include transdermal patches, ointments, lotions, creams, gels, drops, suppositories, sprays, liquids, and powders.

### EXAMPLES

### Example 1

Zebrafish Care and Egg Collection

Standard zebrafish care protocols are described previously (Westerfield, M. 2000). Zebrafish were kept in 3.2 gallon (12 L) and 20 gallon (76 L) polycarbonate tanks at 28° C. The 3.2-gallon tanks housed 25 fish, while the 20-gallon tanks housed 70-100 fish. Tank water was continually cleaned by passing through physical, chemical, biological filtration, and ultraviolet light (UV) irradiation before returning to the tanks at a flow rate of 4-6 gallons/Hr. Remineralized, pH adjusted, and UV sterilized deionized (D.I.) water (system water) was used as source water for the tanks. A saturated solution of Instant Ocean (Aquarium Systems), was injected into a 60 gallon reservoir of D.I. water as needed to remineralize and maintain a conductivity level of 500 μS (microSiemens). A second saturated solution of Sodium Bicarbonate (Sigma-Aldrich S-5761) was injected as needed into the D.I. water to maintain a pH level of 7.2. The system water was then heated to 28° C. and recirculated through a UV sterilizer and added on demand to the zebrafish tanks. A 12-hour dark and 12-hour light day cycle was maintained in the zebrafish facility.

Fish were fed brine shrimp twice a day. The shrimp were filtered through a fine net, rinsed with system water, suspended in system water, and fed to fish. Alternatively, fish could also be fed flake food (Tetra) as a temporary substitute for brine shrimp.

Zebrafish spawning was induced every morning shortly after the start of the light cycle. To collect the eggs, a ‘false bottom container’ system was used (Westerfield, M. 2000). The system consisted of two stackable containers of approximately 2 L, one nested inside the other. The bottom of the inner container was removed and replaced with a stainless steel mesh having holes larger than the eggs and smaller than the fish. The inner container was placed inside the outer container and the setup was filled with system water. Up to 8 zebrafish were placed in the inner container the evening before spawning The following morning when the fish spawned, the eggs fell through the mesh and into the one-inch space between the inner and outer containers and thus could not be reached by the fish and eaten. Fifteen minutes were allowed for spawning, after which time the inner container with the fish was transferred to another outer container. Eggs were collected by filtering the remaining contents of the first outer container through a mesh having holes smaller than the diameter of the eggs. Each group of fish were used for spawning once every two weeks for optimal embryo production.

### Example 2

Identification of a Zebrafish Gene, tde1, Encoding a Membrane Protein

A sequence with strong similarity to the TDE family of predicted membrane proteins, TC62732, was selected from the TIGR Zebrafish Gene Index (ZGI) database. (The Institute for Genomic Research, 2001) This sequence represented the overlap of two zebrafish EST sequences (accession numbers BG302465 and AW133920) and included about 32 nucleotides of the 5′ untranslated region and about 600 nucleotides of the coding sequence. A GenBank search identified a mate-pair (sequence from the opposite end of the same clone) for the AW133920 sequence (accession number AW116276). Using BLAST analysis an additional five EST sequences were identified that match the above sequences (accession numbers BI877046, BI877690, BI709122, BI890769, BI878591). All of the EST sequences were aligned to produce a consensus sequence. The TC62732 partial coding sequence corresponding to the zebrafish tde1 gene was referred to as AN1.

To obtain the full-length zebrafish coding sequence, 3′ Rapid Amplification of mRNA Ends by PCR (RACE) was performed using SuperScript II reverse transcriptase and the suggested manufacturers protocol (Invitrogen). The following primer was used for reverse transcription in the 3′ RACE protocol (V=A, G, OR C):

| | |
|---|---|
| 5′ ACCACTTCCTACAACAAAGCTGGGTTTTTTTTTTTTTTTTTTTTTTV 3′ | (SEQ ID NO.37) |

The following primers were used in the 3′ RACE protocol to determine the full length cDNA sequence for tde1:

| | |
|---|---|
| Primary PCR primer for tde1: | |
| 5′ CATTCATGGAACCAAAAGTGGGTGG 3′ | (SEQ ID NO.38) |
| 5′ CGACAGAACACGCTCCAGCATTGACCACTTCCTACAACAAAGCTGGGT 3′ | (SEQ ID NO.39) |
| 5′ CGACAGAACACGCTCCAGCATTG 3′ | (SEQ ID NO.40) |
| Secondary PCR primer for tde1: | |
| 5′ TGCAGCCCTACTCAGTTTCACATTGG 3′ | (SEQ ID NO.41) |
| 5′ CGCTCCAGCATTGACCACTTCCTAC 3′ | (SEQ ID NO.42) |

The 3′ RACE products were gel isolated and cloned into pCR4/TOPO vector (Invitrogen). Automatic sequencing reactions were performed using primers designed from the cloning vector as follows:

| | |
|---|---|
| M13 Forward(−20): | |
| 5′ GTAAAACGACGGCCAGTG 3′ | (SEQ ID NO.43) |
| M13 Reverse(−27): | |
| 5′ GGAAACAGCTATGACCATG 3′ | (SEQ ID NO.44) |

The amino acid sequence of TDE1 was determined based on a conceptual translation of the coding sequence identified in the full length cDNA and showed 56% sequence identity with both mouse and human TMS1. Percent identity is determined based on a multiple sequence alignment created using the ClustalW algorithm (Thompson, J. D. 1994). The alignment of zebrafish TDE1 with mouse and human TDE family members is shown in FIG. 1. FIG. 2 shows the similarity between zebrafish TDE1 and TDE family members mouse and human. Together these data indicate that zebrafish TDE1 shows structural conservation to human and mouse TDE1 (TMS1) and based on searches of available sequence from human, mouse and zebrafish suggest that TDE1 in zebrafish is the homologue of mammalian TDE1.

### Example 3

Spatial Expression Pattern of Zebrafish tde1 in Early Zebrafish Embryos

To visualize the spatial expression pattern of the tde1 gene throughout zebrafish embryogenesis, whole mount in situ hybridizations were conducted as previously described (Jowett, J. 1999). The zebrafish tde1 gene was labeled with digoxigenin and used as a probe.

The spatial expression pattern of zebrafish tde1 was visualized at different embryonic stages. At 18 hours post-fertilization (hpf), tde1 was expressed in the notochord along the anterior/posterior axis. At 24 hpf, tde1 was expressed in the pronephric ducts, the very posterior notochord, and in a bilateral patch of cells ventrolateral to the notochord. At 48 hpf, tde1 was expressed in the ventral-most portion of the caudal vein posterior to the urogenital opening, the pronephric ducts, right and left pectoral fins, and the epithelium surrounding the olfactory pits. The expression in the pronephic ducts and the bilateral patches of cell ventral to the notochord indicates that tde1 is competent to participate in vessel and capillary formation. Furthermore, expression of tms1 in the mouse brain, testes, kidney and liver tumor has been previously described (Grossman, T. R. 2000). While the testes expression cannot be determined in the zebrafish embryo at the time points analyzed the expression in the pronephic ducts corresponds to the mouse kidney expression and indicates that TDE1 will have a similar role in mammals as in zebrafish.

### Example 4

Morpholino Inactivation of Zebrafish tde1

To determine the function of tde1 in early zebrafish development, morpholino phosphorodiamidate oligonucleotides (morpholinos or MOs) that target the 5′ untranslated region of zebrafish tde1 were generated and used to decrease tde1 gene expression. The zebrafish tde1-MOs had the following sequences:

| | |
|---|---|
| SZ37: | |
| 5′-GGTTCCTCATAATTCCTCAGTCTTC-3′ | (SEQ ID NO:45) |
| SZ126: | |
| 5′-GCTCGTGAAAGCGGAAAATCGC-3′ | (SEQ ID NO:46) |

Morpholinos were obtained from Gene Tools, LLC (Philomath, Oreg.), and were designed to bind to the 5′ untranslated region at or near the initiating methionine. Sequences were selected based on parameters recommended by the manufacturer, such that morpholinos 25 nucleotides in length with approximately 50% G/C and 50% A/T content were generated. Internal hairpins and runs of four consecutive G nucleotides were avoided.

Morpholinos were solubilized in water at a concentration of 8 mM (approximately 65 mg/mL) or 50 mg/mL. The resulting stock solution was diluted to working concentrations of 0.09 to 3 mg/mL in water or 1× Danieau solution. Danieau buffer consisted of 8 mM NaCl, 0.7 mM KCl, 0.4 mM MgSO₄, 0.6 mM Ca(NO₃)₂, and 5.0 mM HEPES (pH 7.6). Zebrafish embryos at the 1 to 4 cell stages were microinjected with 4-9 nL of morpholinos.

The morpholino injection method was very similar to the mRNA injection method described previously (Hyatt, T. M. 1999). The collected eggs were transferred onto agarose plates as described previously (Westerfield, M. 2000). While agarose plates for mRNA injections were kept cold to slow embryo development, the plates for morpholino injections were prewarmed to approximately 20° C., since morpholino injection into cold embryos were found to increase non-specific effects and mortality of the injected embryos.

Needles used for morpholino injections were the same as for mRNA injections (Hyatt, T. M. 1999). The needles were back-filled with a pipette and calibrated by injecting the loaded morpholino solution into a glass capillary tube. The picoinjector volume control was then set up for 1.5 to 15 nL. The injection volume depended on the required dose; 1.5 ng to 18 ng of morpholino usually were injected. Morpholino solutions were injected through the chorion into the yolk of zebrafish embryos. Injected embryos were transferred to petri dishes containing system water and allowed to develop at 28° C.

### Example 5

Morphology of Zebrafish Embryos Injected with tde1-MOs

The phenotypes of zebrafish embryos injected with morpholino-modified polynucleotides were first assessed by visual inspection using dissecting microscopes. Microscopic observation at about 28 hours post-fertilization revealed several abnormalities. Mild cell death in the brain, reduced eye diameter, and hydrocephalic hindbrain, phenotypes associated with general morpholino toxicity, as well as bent body axis were observed in the injected embryos.

At 56 hours, the observed phenotype displayed hydrocephalic hindbrain, reduced eyes, reduced pectoral fin buds, precardial edema, thinner body, and ventrally bent tail. These effects were specific as injection of either SZ37 or SZ126 gave rise to the same phenotype. Coinjection of both morpholinos results in a stronger phenotype. These morpholinos therefore act synergistically in the inhibition of vascular development.

### Example 6

Microangiography Analysis of Zebrafish Embryos Injected with tde1-MOs

To determine whether the vasculature in zebrafish embryos injected with tde1-MOs formed properly, microangiography was performed on both uninjected control embryos and embryos injected with either SZ37 or SZ126, or both. In microangiography, fluorescent FITC-Dextran dye is microinjected into the common cardinal vein of zebrafish embryos as described previously (Nasevicius, A. 2000). Approximately 10 nL of FITC-Dextran fluorescent dye (20 mg/mL) was microinjected into 48-hour embryos incubating in 0.004% Tricain solution. The dye is taken to the heart and then pumped into the systemic circulation, allowing visualization of the entire vasculature using fluorescent microscopy. Results of microangiography showed that embryos injected with either SZ37 or SZ126 exhibited defects in vasculogenesis (initial formation of axial vessels) and angiogenesis (sprouting of new vessels from existing axial vessels). Moreover, coinjection of both morpholinos resulted in more intense loss of vasculature, indicating the specificity of the observed phenotype.

FIG. 3 shows the percentages of embryos exhibiting decreased or no blood vessel formation subsequent to injection with SZ37, SZ126, or both MOs. With regards to FIG. 3, weak phenotype was characterized by some lack of intersomitic blood vessels indicating a defect in angiogenesis as the intersomitic vessels sprout from the axial vasculature. Strong phenotype was characterized by detection of only the heart or heart with head blood vessels but no axial or intersomitic vasculature and indicates defects in vasculogenesis. These results were compiled from the results of two independent experiments. Referring to FIG. 3, for SZ37: the numbers of embryos were n¹=19 and n²=19, and for SZ126: n¹=14 and n²=22; and for (SZ37+SZ126: n¹=14 and n²=19). Taken together, the observations of both weak and strong phenotypes indicate that TDE1 can participate in both vasculogenesis and angiogenesis but with a preferential role in angiogenesis.

### Example 7

Final Differentiation of Endothelial Cells Was Abnormal in tde1-MO Injected Embryos

To determine whether vascular defects observed in embryos injected with tde1-MOs resulted from defects in differentiation of endothelial cells, the expression of vascular endothelial cell marker VE-cadherin (cdh5) was examined using in situ hybridization for zebrafish embryos. This marker is expressed in differentiated vascular endothelial cells throughout vascular development (Breier, G. 1996). The analysis revealed that axial cdh5 expression was normal in tde1-MO injected embryos, indicating that vasculogenesis, which results in formation of axial vasculature, was unperturbed. Intersomitic cdh5 expression, however, was reduced in tde1-MO injected embryos. 66% of embryos exhibited decreased differentiation of vascular endothelial cells subsequent to injection with SZ37, SZ126, or both MOs. 22% of those displayed a weak phenotype characterized by a some lack of intersomitic blood vessel staining. 44% of those displayed a strong phenotype characterized by a complete lack of intersomitic vessel staining. These results were compiled from a single experiment; with SZ37 1 ng, SZ126 1.5 ng, with n=9. This finding indicates that angiogenesis, the remodeling of already-formed vessels and formation of new vessels, was inhibited in tde1-MO injected embryos, resulting in underdevelopment of intersomitic vasculature.

### Example 8

Synergy of tde1-MO and vegf-MO

Signaling by members of the Vascular Endothelial Growth Factor (VEGF) gene family is implicated in the formation of vasculature during embryogenesis, during wound healing, and for the growth of tumor-induced vasculature (Carmeliet, P. 1996; Carmeliet, P. 1997; Ferrara, N. 1999). Since VEGF plays a central role in vasculogenesis and angiogenesis, effects due to decrease in expression from both tde1 and vegf were examined. Zebrafish embryos were injected with two MOs: the tde1-MO (SZ37) and a vegf-MO (5′ GTATCAAATAAACAACCAAGTTCAT 3′, SEQ ID NO. 47). Morpholino injections and zebrafish phenotypic analysis were performed as described in (Nasevicius, A. 2000). FIG. 4 is a bar graph comparing the percentages of embryos exhibiting axial vessel deficiency when injected with tde1-MO alone (TDE1, 0.5 or 1.5 ng), vegf-MO alone (VEGF, 1.5 ng), tde1-MO and vegf-MO (0.5 ng+1.5 ng or 1.5 ng+1.5 ng). These results show that injections of both tde1-MO and vegf-MO had a synergistic effect on the percentages of embryos exhibiting defective axial vessels. Therefore, TDE1 interacts functionally with VEGF, and plays a role in angiogenesis.

With regards to FIG. 4, weak phenotype is characterized by some lack of intersomitic blood vessels indicating a defect in angiogenesis as the intersomitic vessels sprout from the axial vasculature. Strong phenotype was characterized by detection of only the heart or heart with head blood vessels but no axial or intersomitic vasculature and indicates defects in vasculogenesis. These results were compiled from a single experiment. Referring to FIG. 4, tde1-MO 0.5 ng, n=18; tde1-MO 1.5 ng, n=19; vegf-MO 1.5 ng, n=20; tde1-MO 0.5 ng+vegf-MO 1.5 ng, n=19; tde1-MO 1.5 ng+vegf-MO 1.5 ng, n=20. Taken together, the observations of both weak and strong phenotypes indicate that TDE1 and VEGF can participate in both vasculogenesis and angiogenesis but with a preferential role in angiogenesis.

### Example 9

Zebrafish Embryos Injected with tde1-MOs Exhibit Altered Expression of Early and Late Vascular Markers

To determine whether vascular defects observed in embryos injected with tde1-MOs resulted from defects in specification and/or patterning of vascular endothelial cells, the expression of known vascular genes was analyzed by in situ hybridization. The genes for this analysis include flk-1 (Fouquet, B. 1997), fli-1 and flt-4 (Thompson, 1998), which play a role in the early specification of vascular endothelial cells, and tie-1 and tie-2 (Puri, M. C. 1999), which are implicated in the maturation and maintenance of the vasculature. In normal zebrafish embryos flt-4 and tie-2 expression is only observed in the forming axial vessels and not the intersomitic vessels, suggesting a role in vasculogenesis rather than angiogenesis. The expression of these genes was examined in uninjected control embryos and embryos injected with tde1-MOs (FIG. 5). Results showed that expression of flk-1 (77% n=13), fli-1 (94% n=16) and tie-1 (71% n=14) were lost in the forming intersomitic vessels of tde1-MO injected embryos. flt-4 (n=15) and tie-2 (n=12) axial vessel expression was normal in tde1-MO injected embryos. Therefore, the loss of flk-1, fli-1 and tie-1 expression in the forming intersomitic vessels of tde1-MO injected embryos indicates a lack of intersomitic vascular specification and maturation.

With regards to FIG. 5, weak phenotype is characterized by some lack of intersomitic blood vessels and strong phenotype is characterized by a complete lack of intersomitic blood vessels. These results were compiled from a single experiment on embryos co-injected with SZ37 (1 ng) and SZ126 (1.5 ng).

### Example 10

Identification of a Zebrafish Gene ptv Encoding a Novel Membrane Protein

A sequence with strong similarity to a human 30 kDa protein (accession number AAF67487), TC60904, was selected from the TIGR Zebrafish Gene Index (ZGI) database. (The Institute for Genomic Research, 2001) This sequence represented the overlap of two zebrafish EST sequences (accession numbers AW17535 and AW305855) and included about 117 nucleotides of the 5′ untranslated region and about 663 nucleotides of the coding sequence. An additional EST sequence was identified as the mate-pair (sequence from the opposite end of the same clone) for the AW305855 sequence (accession number AW419481). The partial cDNA sequence corresponding to the zebrafish ptv gene was named AN2. For the sake of convenience, the name ptv is used to indicate all species and is used interchangeably with an2.

To obtain the full-length zebrafish ptv coding sequence, RT-PCR reactions were performed using primers designed from the sequences described above. The primer designed to the 5′ end of the coding sequence contains a two-nucleotide mismatch (lowercase) in order to allow directional cloning of the resulting fragment into the pENTR/D-TOPO vector (Invitrogen). The following primers were used to obtain the complete sequence of the ptv cDNA:

| | |
|---|---|
| 5′ cAcCATGGCTGAGCCCGAGCTCCTC 3′ | (SEQ ID NO.48) |
| 5′ CCAACACCTCTCAGTAGTGAACAGGCAC 3′ | (SEQ ID NO.49) |
| 5′ TGCAGATCAGTCTCGCATTATGCAGG 3′: | (SEQ ID NO.50) |
| 5′ GCCACCATGCCACCTAGCTCAACTAA 3′ | (SEQ ID NO.51) |

RT-PCR products were gel isolated and cloned into pCR4/TOPO (Invitrogen) vector. Automatic sequencing reactions were performed using primers designed from the cloning vector as follows:

| | |
|---|---|
| M13 Forward(−20): | |
| 5′ GTAAAACGACGGCCAGTG 3′ | (SEQ ID NO.52) |
| M13 Reverse(−27): | |
| 5′ GGAAACAGCTATGACCATG 3′ | (SEQ ID NO.53) |

The amino acid sequence of zebrafish PTV was determined based on a conceptual translation of the open reading frame identified in the full length cDNA and showed 90% sequence identity with both mouse 0610039A15Rik and human LOC55831. Percent identity is determined based on a multiple sequence alignment created using the ClustalW algorithm (Thompson, J. D. 1994). The alignment of zebrafish PTV with mouse and human sequences is shown in FIG. 6. FIG. 7 shows the similarity between PTV from zebrafish and other species. Together these data indicate that zebrafish PTV shows structural conservation to human and mouse PTV and based on searches of available sequence from human, mouse and zebrafish suggest that PTV in zebrafish is the homologue of mammalian PTV.

### Example 11

Spatial Expression Pattern of Zebrafish ptv in Early Zebrafish Embryos

To visualize the spatial expression pattern of ptv throughout zebrafish embryogenesis, whole mount in situ hybridizations were conducted as described in (Jowett, J. 1999). The zebrafish ptv gene was labeled with digoxigenin and used as a probe.

The spatial expression pattern of zebrafish ptv was visualized at different embryonic stages. At 18 hpf, ptv was expressed ubiquitously. At 24 hpf, ptv was expressed in the brain, bilateral patches of ventrolateral cells near the body, pronephric ducts and cells in the tail ventral to the pronephric ducts. ptv was also expressed in the posterior-most portion of the floor plate. At 48 hpf, ptv was expressed throughout the head. The expression of ptv at 24 hpf is consistent with a role in vasculogenesis and angiogenesis.

### Example 12

Morpholino Inactivation of Zebrafish ptv

To determine the function of ptv in early zebrafish development, morpholinos (MOs) that target the 5′ untranslated region of zebrafish ptv were generated and used to decrease ptv gene expression. Except as otherwise stated, the procedures of Example 4 were followed. The zebrafish ptv-MOs had the following sequences:

| | |
|---|---|
| SZ18: | |
| 5′-CCCTGCTCTCGTATTCAAATGACGG-3′ | (SEQ ID NO:54) |
| S767: | |
| 5′-ACCGATAACGACTCGAATCAGGATG-3′ | (SEQ ID NO:55) |

### Example 13

Morphology of Zebrafish Embryos Injected with ptv-MOs

The phenotypes of zebrafish embryos injected with morpholino-modified polynucleotides were first assessed by visual inspection using dissecting microscopes. Microscopic observations showed that the overall morphology of embryos injected with ptv-MOs was relatively normal at about 28 hours post-fertilization, as well as 56 hours post-fertilization. The same phenotype was observed upon injection of either SZ18 or SZ67, as well as coinjection of both of these morpholinos. These results indicate that the phenotype is specific.

### Example 14

Microangiography Analysis of Zebrafish Embryos Injected with ptv-MOs

To determine whether the vasculature in zebrafish embryos injected with ptv-MOs formed properly, microangiography (see Example 6) was performed on both uninjected control embryos and embryos injected with either SZ18, SZ67, or both. Results of microangiography showed that embryos injected with either SZ18 or SZ67 exhibited defects in angiogenesis (sprouting of new vessels from existing axial vessels), but not vasculogenesis (initial formation of axial vessels). Moreover, coinjection of both morpholinos resulted in more intense loss of vasculature, indicating the specificity of the observed phenotype.

FIG. 8 shows the percentages of embryos exhibiting decreased or no blood vessel formation subsequent to injection with either SZ18, SZ67, or both MOs. In regard to FIG. 8, weak phenotype was characterized by some lack of intersomitic blood vessels indicating a defect in angiogenesis as the intersomitic vessels sprout from the axial vasculature. Strong phenotype was characterized by detection of only the heart or heart with head blood vessels but no axial or intersomitic vasculature and indicates defects in vasculogenesis. These results were compiled from a single experiment. Referring to FIG. 8, SZ18 6 ng, n=17; SZ67 9 ng, n=16; SZ18 6 ng+SZ67 9 ng, n=16. Taken together, the observations of both weak and strong phenotypes indicate that PTV can participate in both vasculogenesis and angiogenesis but with a preferential role in angiogenesis.

### Example 15

Final Differentiation of Endothelial Cells was Abnormal in ptv-MO Injected Embryos

To determine whether vascular defects observed in embryos injected with ptv-MOs resulted from defects in differentiation of endothelial cells, the expression of vascular endothelial cell marker VE-cadherin (cdh5) was examined using in situ hybridization (see Example 7). Analysis revealed that axial cdh5 expression was normal in ptv-MO injected embryos, indicating that vasculogenesis, which results in formation of axial vasculature, was unperturbed. Intersomitic cdh5 expression, however, was reduced in ptv-MO injected embryos. This finding indicates that angiogenesis, the remodeling of already formed vessels and formation of new ones, was inhibited in ptv-MO injected embryos, resulting in underdevelopment of intersomitic vasculature. 73% of embryos exhibited decreased differentiation of vascular endothelial cells subsequent to injection with SZ18, SZ67, or both MOs. 18% of those displayed a weak phenotype characterized by some lack of intersomitic blood vessel staining. 55% of those displayed a strong phenotype characterized by a complete lack of intersomitic vessel staining. These results were compiled from a single experiment. (SZ18 6 ng+SZ67 9 ng: n=11). This finding indicates that angiogenesis, the remodeling of already formed vessels and formation of new ones, was inhibited in ptv-MO injected embryos, resulting in underdevelopment of intersomitic vasculature.

### Example 16

Synergy of ptv-MO and vegf-MO

Signaling by members of the Vascular Endothelial Growth Factor (VEGF) gene family is implicated in the formation of vasculature during embryogenesis, during wound healing, and for the growth of tumor-induced vasculature. Since VEGF plays a central role in vasculogenesis and angiogenesis, effects due to decrease in expression from both ptv and vegf were examined. Zebrafish embryos were injected with two MOs: a ptv-MO (SZ18) and a vegf-MO (5′ GTATCAAATAAACAACCAAGTTCAT 3′, SEQ ID NO. 18). Morpholino injections and zebrafish phenotypic analysis were performed as described previously (Nasevicius, A. 2000).

FIG. 9 is a bar graph comparing the percentages of embryos exhibiting axial vessel deficiency when injected with ptv-MO alone (3 ng or 6 ng), vegf-MO alone (1.5 ng), ptv-MO and vegf-MO (3 ng+1.5 ng or 6 ng+1.5 ng). Referring to FIG. 9, weak phenotype was characterized by some lack of intersomitic blood vessels indicating a defect in angiogenesis as the intersomitic vessels sprout from the axial vasculature. Strong phenotype was characterized by detection of only the heart or heart with head blood vessels but no axial or intersomitic vasculature and indicates defects in vasculogenesis. These results were compiled from a single experiment. Referring to FIG. 9, SZ18 3 ng, n=23; SZ18 6 ng, n=23; vegf-MO 1.5 ng, n=24; SZ18 3 ng+vegf-MO 1.5 ng, n=25; SZ18 6 ng+vegf-MO 1.5 ng, n=24. These results show that injections of both ptv-MO and vegf-MO had a synergistic effect on the percentages of embryos exhibiting defective axial vessels. Therefore, PTV interacts functionally with VEGF, and can play a role in vasculogenesis and angiogenesis.

### Example 17

Expression Analysis: Zebrafish Embryos Injected with ptv-MOs Exhibit Altered Expression of Early and Late Vascular Markers

To determine whether vascular defects observed in embryos injected with ptv-MOs resulted from defects in specification and/or patterning of vascular endothelial cells, the expression of known vascular genes was analyzed by in situ hybridization. The genes for this analysis include flk-1 (Fouquet, B. 1997), fli-1 and flt (Thompson, M. A. 1998), which play a role in the early specification of vascular endothelial cells, and tie-1 and tie-2 (Puri, M. C. 1999), which are implicated in the maturation and maintenance of the vasculature. In normal zebrafish embryos flt-4 and tie-2 expression are only observed in the forming axial vessels and not the intersomitic vessels, suggesting a role in vasculogenesis rather than angiogenesis=. The expression of these genes was examined in uninjected control embryos and embryos injected with ptv-MOs (FIG. 10). Referring to FIG. 10, weak phenotype is characterized by some lack of intersomitic blood vessels and strong phenotype is characterized by a complete lack of intersomitic blood vessels. These results were compiled from a single experiment on embryos co-injected with SZ18 (6 ng) and SZ126 (9 ng). Results showed that expression of flk-1 (71% n=14), fli-1 (67% n=15) and tie-1 (80% n=15) was lost in the forming intersegmental vessels of ptv-MO injected embryos. flt-4 (n=12) and tie-2 (n=15) expression in the axial vessels was normal in ptv-MO injected embryos. Therefore, the loss of flk-1, fli-1 and tie-1 expression in the forming intersomitic vessels of ptv-MO injected embryos indicates a lack of intersomitic vascular specification and maturation.

### Example 18

Identification of a Zebrafish Gene, msn, Encoding a MOESIN Protein

A sequence with strong similarity to human moesin, TC58457, was selected from the TIGR Zebrafish Gene Index (ZGI) database. (The Institute for Genomic Research, 2001). This sequence represented the overlap of two zebrafish EST sequences (accession numbers AI331715 and AI617727) and included about 250 nucleotides of the 5′ untranslated region and about 270 nucleotides of the coding sequence. An additional sequence, TC60664, was identified as containing the mate-pair (sequence from the opposite end of the same clone) for the AI331715 sequence (accession number AI331743). This sequence represented the overlap of AI331743 and eight additional EST sequences (accession numbers AI353701, BF937912, AI497003, AI332263, AI794199, AI331186, AI958444, AI396788). Using BLAST analysis an additional nineteen EST sequences were identified that match the above sequences (accession numbers: BQ092129, BI879428, BQ133095, BI842929, BM777838, AI396788, BM141303, BI890226, AL918485, BQ260755, BI887739, BI890773, AL715615, BM025416, BM155036, BI840137, BI839995, BM777532, BQ261054, AW454351). All of the EST sequences were aligned to produce a consensus sequence. The full-length coding sequence (SEQ ID NO. 2) corresponding to the zebrafish moesin gene was named msn, also referred to herein as moesin or srp1.

To confirm the full-length zebrafish msn coding sequence, RT-PCR reactions were performed using primers designed from the SRP1 consensus sequence described above. The primer designed to the 5′ end of the coding sequence contains a two-nucleotide mismatch (lowercase) in order to allow directional cloning of the resulting fragment into the pENTR/D-TOPO vector (Invitrogen). The following primers were used to confirm the complete sequence of the hke4 open reading frame:

| | |
|---|---|
| 5′ CAccATGCCGAAAACGATCAGTGTTCGT 3′ | (SEQ ID NO.57) |
| 5′ CCTGGTTCTTCATCTGGCTCTCCGA 3′ | (SEQ ID NO.58) |
| 5′ TCTACTGCCCTCCTGAGACTGCGGT 3′: | (SEQ ID NO.59) |
| 5′ CCTGGTTCTTCATCTGGCTCTCCGA 3′ | (SEQ ID NO.60) |

RT-PCR products were gel isolated and cloned into pCR4/TOPO (Invitrogen) vector. Automatic sequencing reactions were performed using primers designed from the cloning vector as follows:

| | |
|---|---|
| M13 Forward(−20): | |
| 5′ GTAAAACGACGGCCAGTG 3′ | (SEQ ID NO. 61) |
| M13 Reverse(−27): | |
| 5′ GGAAACAGCTATGACCATG 3′ | (SEQ ID NO. 62) |

The amino acid sequence of MSN was determined based on a conceptual translation of the open reading frame identified in the full-length cDNA and showed 84% sequence identity with both mouse and human moesin. Percent identity is determined based on a multiple sequence alignment created using the ClustalW algorithm (Thompson, J. D. 1994). The alignment of zebrafish MSN with mouse and human ERM family members is shown in FIG. 11. The percent amino acid comparison to ERM family members is shown in FIG. 12. Together these data indicate that zebrafish MOESIN shows structural conservation to human and mouse MOESIN and based on searches of available sequence from human, mouse and zebrafish suggest that MOESIN in zebrafish is the homologue of mammalian MOESIN.

### Example 19

Spatial Expression Pattern of Zebrafish msn in Early Zebrafish and Mouse Embryos

To visualize the spatial expression pattern of msn throughout zebrafish embryogenesis, whole mount in situ hybridizations were conducted as described in (Jowett, J. 1999). The zebrafish msn gene was labeled with digoxigenin and used as a probe.

The spatial expression pattern of zebrafish msn was visualized at different embryonic stages. At 24 hpf, zebrafish msn was expressed in the axial vessels, intersomitic vessels, vessels in the head, the heart tube, the pharyngeal area and the floor plate. At 48 hpf, zebrafish msn was expressed in the axial vessels, the intersomitic vessels, gut vessels, the heart and pectoral fin buds. At 4 dpf, zebrafish msn was expressed in the head and the heart.

To visualize the spatial expression pattern of msn throughout the early development of the mouse embryo, whole mount in situ hybridizations were as described in (Shen, M. M. 2001). The mouse msn gene was labeled with digoxigenin and used as a probe. The spatial expression pattern of mouse msn was visualized at 8.5 dpf, and was expressed in the axial and intersomitic vessels.

While the expression of moesin in the vascular endothelium of adult mouse (Berryman, M. 1993) and adult human tissues (Johnson, M. W. 2001) has been documented this shows that the vascular expression profile of moesin in zebrafish and mouse embryos is the same and indicates the functional orthology of the zebrafish, mouse, and human moesin.

### Example 20

Morpholino Inactivation of Zebrafish msn

To determine the function of msn in early zebrafish development, morpholinos (MOs) that target the 5′ untranslated region of zebrafish tde1 were generated and used to decrease msn gene expression. This example followed the procedures of Example 4 were performed essentially as described therein unless otherwise stated. The zebrafish msn-MOs had the following sequences:

| | |
|---|---|
| SZ12: | |
| 5′-CGGCATTTTGTCGGTATCTGGTCTC-3′ | (SEQ ID NO:63) |
| SZ84: | |
| 5′-ACGAATGTGTCACAAACTGAAGCTG-3′ | (SEQ ID NO:64) |

### Example 21

Morphology of Zebrafish Embryos Injected with msn-MOs

The phenotypes of zebrafish embryos injected with morpholino-modified polynucleotides were first assessed by visual inspection using dissecting microscopes. Microscopic observations showed that the overall morphology of embryos injected with msn-MO was relatively normal at about 28 hours post-fertilization. At 56 hours post-fertilization an enlarged pericardial sack and reduced blood circulation was observed. The same phenotype was observed upon injection of either SZ12 or SZ84, as well as coinjection of both of these morpholinos, indicating the specificity of the observed phenotype.

### Example 22

Microangiography Analysis of Zebrafish Embryos Injected with msn-MOs

To determine whether the vasculature in zebrafish embryos injected with msn-MOs formed properly, microangiography was performed on both uninjected control embryos and embryos injected with either SZ12, SZ84, or both. In microangiography, fluorescent FITC-Dextran dye is microinjected into the common cardinal vein of zebrafish embryos as described in Nasevicius et al. (2000) Yeast 17:294-301. Approximately 10 nL of FITC-Dextran dye is microinjected into the common cardinal vein of zebrafish embryos as described in previously (Nasevicius, A. 2000). Approximately 10 nL of FITC-Dextran dye is microinjected into the common cardinal vein of zebrafish embryos. The dye is taken to the heart and then pumped into the systemic circulation, allowing visualization of the entire vasculature using fluorescent microscopy. Results of microangiography showed that embryos injected with either SZ12 or SZ84 exhibited defects in both vasculogenesis (initial formation of axial vessels) and angiogenesis (sprouting of new vessels from existing axial vessels). Moreover, coinjection of both morpholinos resulted in more intense loss of vasculature, indicating the specificity of the observed phenotype.

FIG. 13 shows the percentages of embryos exhibiting decreased or no blood vessel formation subsequent to injection with SZ12, SZ84, or both MOs. Weak phenotype is characterized by some lack of intersomitic blood vessels and strong phenotype is characterized by detection of only the heart or heart with head blood vessels but no axial or intersomitic vasculature. These results were compiled from the results of a single experiment, with SZ12, n=24; SZ84, n=20 and SZ12+SZ84, n=30.

### Example 23

Final Differentiation of Endothelial Cells Was Abnormal in msn-MO Injected Embryos

To determine whether vascular defects observed in embryos injected with msn-MOs resulted from defects in differentiation of endothelial cells, the expression of vascular endothelial cell marker VE-cadherin (cdh5) was examined using in situ hybridization. This marker is expressed in differentiated vasculature endothelial cells throughout vascular development. The analysis revealed that axial cdh5 expression was normal in msn-MO injected embryos, indicating that vasculogenesis, which results in formation of axial vasculature, was unperturbed. Intersomitic cdh5 expression, however, was reduced in msn-MO injected embryos. 40% of embryos exhibited decreased differentiation of vascular endothelial cells subsequent to injection with SZ12, SZ84, or both MOs. 27% of those displayed a weak phenotype characterized by some lack of intersomitic blood vessel staining. 13% of those displayed a strong phenotype characterized by a complete lack of intersomitic vessel staining. These results were compiled from a single experiment. (SZ12 3 ng+SZ84 12 ng: n=15). This finding indicates that angiogenesis, the remodeling of already-formed vessels and formation of new ones, was inhibited in tde1-MO injected embryos, resulting in underdevelopment of intersomitic vasculature.

### Example 24

Zebrafish Embryos Injected with msn-MOs Exhibit Altered Expression of Early and Late Vascular Markers

To determine whether vascular defects observed in embryos injected with msn-MO resulted from defects in specification and/or patterning of vascular endothelial cells, the expression of known vascular genes was analyzed by in situ hybridization. The genes for this analysis include flk-1 (Fouquet, B. 1997), fli-1 and flt-4 (Thompson, M. A. 1998), which play a role in the early specification of vascular endothelial cells, and tie-1 and tie-2 (Puri, M. C. 1999), which are implicated in the maturation and maintenance of the vasculature. In normal zebrafish embryos flt-4 and tie-2 expression are only observed in the forming axial vessels and not the intersomitic vessels, suggesting a role in vasculogenesis rather than angiogenesis. The expression of these genes was examined in uninjected control embryos and embryos injected with msn-MOs (FIG. 14). Results showed that flk-1 (n=15) and fli-1 (n=15) expression was normal in msn-MO injected embryos, including the expression within the forming intersomitic vessels. The expression of tie-1 (50% n=14) was lost in the forming intersomitic vessels of msn-MO injected embryos. flt-4 (n=15) and tie-2 (n=10) expression in the axial vessels was normal in msn-MO injected embryos. Therefore, the loss of tie-1 expression in the forming intersomitic vessels of msn-MO injected embryos indicates a lack of intersomitic vascular specification. Referring to FIG. 14, weak phenotype is characterized by some lack of intersomitic blood vessels and strong phenotype is characterized by a complete lack of intersomitic blood vessels. These results were compiled from a single experiment on embryos co-injected with SZ12 (3 ng) and SZ84 (12 ng).

### Example 25

Identification of a hke4 Encoding a Membrane Protein

A sequence with strong similarity to human HKE4, AF196345, was selected from GenBank. This sequence includes about 142 nucleotides of the 5′ untranslated region and about 1057 nucleotides of the coding sequence. Using BLAST analysis twenty EST sequences were identified that match the above sequence (accession numbers BQ480733, BM776127, BM777475, BM095389, BM777787, BM775136, BQ783998, BI984965, BI984358, AI722990, BI886108, BM095736, BI979712, AI437101, AM721488, BI980324, BG985639, AI416347, AW453952, BQ075798). All of the EST sequences were aligned to produce a consensus sequence representing the full-length zebrafish hke4 cDNA sequence. The consensus EST sequence corresponding to the zebrafish hke4 gene was referred to as AN3.

To confirm the full-length zebrafish hke4 coding sequence (SEQ ID NO. 2), RT-PCR reactions were performed using primers designed from the AN3 sequence described above. The primer designed to the 5′ end of the coding sequence contains a two-nucleotide mismatch (lowercase) in order to allow directional cloning of the resulting fragment into the pENTR/D-TOPO vector (Invitrogen). The following primers were used to confirm the complete sequence of the hke4 open reading frame:

| | |
|---|---|
| 5′ cACcATGAGGGTCTTTAGCAAACGCTATT 3′ | (SEQ ID NO.65) |
| 5′ TCATACTCTGCAATCAGCACCATCA 3′ | (SEQ ID NO.66) |
| 5′ ATTCTTGTCCAATCAGGCTGCACCAA 3′: | (SEQ ID NO.67) |
| 5′ GGTCACTGCGCCTCATATTCCAAGTT 3′ | (SEQ ID NO.68) |

RT-PCR products were gel isolated and cloned into pCR4/TOPO (Invitrogen) vector. Automatic sequencing reactions were performed using primers designed from the cloning vector as follows:

| | |
|---|---|
| M13 Forward(−20): | |
| 5′ GTAAAACGACGGCCAGTG 3′ | (SEQ ID NO.69) |
| M13 Reverse(−27): | |
| 5′ GGAAACAGCTATGACCATG 3′ | (SEQ ID NO.70) |

The amino acid sequence of zebrafish HKE4 was determined based on a conceptual translation of the open reading frame identified in the full length cDNA and showed 50% sequence identity with both mouse and human HKE4. Percent identity is determined based on a multiple sequence alignment created using the ClustalW algorithm (Thompson, J. D. 1994). The alignment of zebrafish HKE4 with mouse and human HKE4 sequences is shown in FIG. 15. The percent amino acid comparison to HKE4 homologues is shown in FIG. 16. Together these data indicate that zebrafish HKE4 shows structural conservation to human and mouse HKE4 and based on searches of available sequence from human, mouse and zebrafish suggest that HKE4 in zebrafish is the homologue of mammalian HKE4.

### Example 26

Spatial Expression Pattern of hke4 in Early Zebrafish and Mouse Embryos

To visualize the spatial expression pattern of hke4 throughout zebrafish embryogenesis, the procedures of Example 3 were performed essentially as described therein unless otherwise stated. The zebrafish hke4 gene was labeled with digoxigenin and used as a probe.

The spatial expression pattern of zebrafish hke4 was visualized at different embryonic stages. At 18 hpf, hke4 was expressed in the hypochord and floor plate along the midline and in the posterior portion of the notochord. At 24 hpf, hke4 was expressed along the tail axis just dorsal from the pronephric ducts and in the posterior portion of the notochord. At 48 hpf, hke4 was expressed in the anterior portion of the gut, in the area of the pharynx, pharyngeal endoderm and pectoral fin bud cells. At 4 dpf, hke4 was expressed in the tissues surrounding the pharyngeal arches, the pancreas and liver. Zebrafish hke4 is expressed in tissues that play a role in chondrogenesis and bone formation.

At 9.5 days post-fertilization (dpf) of mouse development hke4 was expressed in lateral mesoderm of the fore- and hind limbs. The cells that express hke4 in the mouse limbs participate in cartilage and bone development. The similar expression of hke4 in regions of cartilage and bone development indicates a similar function for HKE4 in all vertebrate organisms including humans.

### Example 27

Morpholino Inactivation of Zebrafish hke4

To determine the function of hke4 in early zebrafish development, morpholinos (MOs) that target the 5′ untranslated region of zebrafish hke4 were generated and used to decrease hke4 gene expression. The procedures of Example 4 were performed essentially as described therein unless otherwise stated. The zebrafish hke4-MOs had the following sequences:

| | |
|---|---|
| SZ38: | |
| 5′-AGCGATTTGCTAAAGACCCTCATTG-3′ | (SEQ ID NO:71) |
| SZ83: | |
| 5′-GCAATCTGCTAACCGCATCCACGTC-3′ | (SEQ ID NO:72) |

### Example 28

Morphology of Zebrafish Embryos Injected with hke4-MOs

The phenotypes of zebrafish embryos injected with morpholinos were first assessed by visual inspection with a dissecting microscope. At about 24 hours post-fertilization, embryos appeared morphologically normal. However, at 4 days post fertilization (dpf), zebrafish larvae lacked jaws as compared to control embryos. When SZ38 and SZ83 were injected together, slightly more than 40% of the surviving zebrafish larvae lacked jaws. In separate studies, the effect of the hke4 morpholinos was specific, as injection of either SZ38 or SZ83 gave rise to the same phenotype. Furthermore, injection of 39 different MOs did not result in any embryos with jaw defects. These morpholinos therefore act synergistically in the inhibition of jaw formation and are expected to synergistically inhibit cartilage and bone formation.

### Example 29

Alcian Blue Cartilage Staining of Zebrafish Embryos Injected with hke4-MO

To determine whether the cartilages in zebrafish embryos injected with hke4-MOs formed properly, alcian blue staining was performed on both uninjected control embryos and embryos injected with hke4-MOs at 5 dpf as described previously (Tatjana, P. 1996). FIG. 17 shows the results of alcian blue staining. Embryos injected with either SZ38 or SZ83 exhibited defects in cartilage formation. Moreover, coinjection of both morpholinos resulted in more intense loss of cartilage, indicating the specificity of the observed phenotype. Referring to FIG. 17, weak phenotype is characterized by a misshapen pattern of the jaw cartilage and strong phenotype is characterized by a lack jaw cartilage with reduced and/or misshapen cartilage in the rest of the embryo. These results were compiled from the results of a single experiment, with SZ38 n=14; SZ83 n=7; and SZ38+SZ83: n=6.

### Example 30

Expression of Neural Crest Markers after Injection with hke4-MOs

To determine whether cartilage defects observed in embryos injected with hke4-MOs resulted from defects in formation or patterning of neural crest cells, the expression of the neural crest cell markers distal-less homeo box 2 (dlx2) and forkhead 6 (fkd6) were examined using in situ hybridization. The zebrafish dlx2 marker is expressed shortly after gastrulation in the ventral forebrain rudiment and the hindbrain neural crest cells and can be used to specify pattern formation or cell fate determination in the forebrain, in peripheral structures of the head, and in the fins (Akimenko, M. A. 1994). The zebrafish jkd6 gene is a marker for premigratory neural crest cells (Kelsh, R. N. 2000). The analysis revealed that neural crest cell expression of both dlx2 and fkd6 was normal in hke4-MO injected embryos, indicating that the defects in cartilage formation are not a result of neural crest cell defects.

### Example 31

Calcein Staining of Zebrafish Larvae after Injection with hke4-MOs

To determine whether the bone in zebrafish embryos injected with hke4-MOs formed properly, calcein staining was performed on both uninjected control embryos and embryos injected with hke4-MOs at 7 dpf (Du, 2001). FIG. 18 shows the results of calcein staining. Embryos injected with either SZ38 or SZ83 exhibited defects in bone formation. Moreover, coinjection of both morpholinos resulted in more intense loss of bone, indicating the specificity of the observed phenotype. Taken together, these results indicate a role for HKE4 in both cartilage and bone formation. Referring to FIG. 18, weak phenotype is characterized by a lack of staining of the jawbones but normal range of vertebrae staining and strong phenotype is characterized by little or no bone staining including a lack of vertebral staining. These results were compiled from the results of two independent experiments, with SZ38 n1=20 and n2=9; SZ83: n1=20 and n2=10; SZ38+83: n1=11 and n2=11.

References

1. Abe, K., et al. Searching for coding sequences in the mammalian genome: The H-2K region of the mouse MHC is replete with genes expressed in embryos. EMBO J. 7, 3441-3449 (1988).

2. Akimenko, M. A., et al. Combinatorial expression of three zebrafish genes related to distal-less: part of a homeobox gene code for the head. J. Neurosci. 14, 3475-3486 (1994).

3. Anatipour, M., et al. Search for MHC-associated genes in human: Five new genes centromeric to HLA-DP with yet unknown functions. Immunogentics 35, 272-278 (1992).

4. Ando, A., et al. cDNA cloning of the human homologues of the mouse Ke4 and Ke6 genes at the centromeric end of the human MHC region. Genomics 35, 600-602 (1996).

5. Berryman, M., Franck, Z., and Bretscher, A. Ezrin is concentrated in the apical microvilli of a wide variety of epithelial cells whereas moesin is found primarily in endothelial cells. J. Cell Sci. 105, 1025-1043 (1993).

6. Bolton, E. T. et al. A General Method for the Isolation of RNA Complementary to DNA. Proc. Natl. Acad. Sci. 48, 1390 (1962).

7. Bossolasco, M. et al. The human TDE gene homologue: Localization to 20q13.1-13.3 and variable expression in human tumor cell lines and tissue. Mol. Carcinog. 26, 189-200 (1999).

8. Breier, G. G., et al., Molecular cloning and expression of murine vascular endothelial-cadherin in early stage development of the cardiovascular system. Blood 87, 630-641 (1996).

9. Carmeliet, P., et al. Abnormal blood vessel development and lethality in embryos lacking a single VEGF allele. Nature 380, 435-439 (1996).

10. Cook, Antisense Medicinal Chemistry, Springer, New York, pp. 51-101 (1998).

11. Ferrara, N. Molecular and biological properties of vascular endothelial growth factor. J. Mol. Med. 77, 527-543 (1999).

12. Fouquet, B. B., et al., Vessel patterning in the embryo of the zebrafish: guidance by notochord. Dev. Biol. 183, 37-48 (1997).

13. Froehler, B., et al., Phophoramidate analogues of DNA: synthesis and thermal stability of heteroduplexes. Nucleic Acids Res. 16, 4831-4839 (1988).

14. Funayama, N., et al. Radixin is a novel member of the band 4.1 family. J. Cell Biol. 115, 1039-1048 (1991).

15. Gould, K. L., et al. cDNA cloning and sequencing of the protein-tyrosine kinase substrate, ezrin, reveals homology to band 4.1. EMBO J. 8, 4133-4142 (1989).

16. Grossman, T. R., Luque, J. M., and Nelson, N. Identification of a ubiquitous family of membrane proteins and their expression in the mouse brain. J. Exp. Biol. 203, 447-457 (2000).

17. Haase, V. H., et al. The murine NF2 homologue encodes a highly conserved merlin protein with alternative forms. Hum. Mol. Genet. 3, 407-411 (1994).

18. Hill, K. K. Genetic interactions between the Drosophila Abelson (Abl) tyrosine kinase and failed axon connections (fax), a novel protein in axon bundles. Genetics 141, 595-606 (1995

19. Huynh, D. P., et al. Alternative transcripts in the mouse neurofibromatosis type 2 (NF2) gene are conserved and code for schwannomins with distinct C-terminal domains. Hum. Mol. Genet. 3, 1075-1079 (1994).

20. Hyatt, T. M. et al., Vectors and techniques for ectopic gene expression in zebrafish. Methods Cell Biol., 59, 117-126 (1999).

21. Janatipour, M., et al. Search for MHC-associated genes in human: Five new genes centromeric to HLA-DP with yet unknown functions. Immunogentics 35, 272-278 (1992).

22. Johnson, M. W., Miyata, H., and Vinters, H. V. Ezrin and moesin expression within the developing human cerebrum and tuberous sclerosis-associated cortical tubers. Acta Neuropathol. 104, 188-196 (2002).

23. Jowett, J. J. Analysis of protein and gene expression. Methods Cell Biol. 59, 63-85 (1999).

24. Kelsh, R. N., et al. Expression of zebrafish fkd6 in neural crest-derived glia. Mech. Dev. 93, 161-164 (2000).

25. Krueger, W. H. H., et al. TPO1, a member of a novel protein family, is developmentally regulated in cultured oligodendrocytes. J. Neurochem. 69, 1343-1355 (1997).

26. Lankes, W. T. and Furthmayr, H. Moesin: a member of the protein 4.1-talin-ezrin family of proteins. Proc. Natl Acad. Sci. 88, 8297-8301 (1991).

27. Lasswell, J., et al. Cloning and characterization of IAR1, a gene required for auxin conjugate sensitivity in Arabadopsis. Plant Cell 12, 2395-2408 (2000).

28. Lebel, M. and Mes-Masson, A. M. Sequence analysis of a novel cDNA which is overexpressed in testicular tumors from polyomavirus large T-antigen transgenic mice. DNA Seq. 5, 31-39 (1994).

29. Martoglio, A. et al. Changes in tumorigenesis- and angiogenesis-related gene transcript abundance profiles in ovarian cancer detected by tailored high density cDNA arrays. Mol. Med. 6, 750-765 (2000).

30. Miller, P. S., et al., Nonionic nucleic acid analogues. Synthesis and characterization of dideoxyribonucleosid methylphoshonates. Biochemistry 18, 5134-5143 (1979).

31. Miller, P. S., et al., Oligothymidylate analogues having stereoregular, alternating methylphosphonate/phosphodiester backbones. Synthesis and physical studies. J. Biol. Chem. 255, 9659-9665 (1980).

32. Nasevicius, A., et al., Distinct requirements for zebrafish angiogenesis revealed by a VEGF-A morphant. Yeast 17, 294-301(2000).

33. Nelissen, B., DeWachter, R. and Goffeau, A. Classification of all putative permeases and other membrane plurispanners of the major facilitator superfamily encoded by the complete genome of Saccharomyces cerevisiae. FEMS Microbiol. Rev. 21, 113-134 (1997).

34. Nielsen, P. E., et al., Sequence-selective recognition of DNA by strand displacement with a thymine-substituted polyamide. Science 254, 1497-1500 (1991).

35. Pfam—A database of protein domain family alignments and HMMs Copyright© 1996-2002, The Pfam Consortium.

36. Pitha, J., et al., Poly(1-vinyluracil): the preparation and interactions with adenosine derivatives. Biochim Biophus. Acta 204, 39-48 (1970).

37. Pitha, P. M., et al., Preparation and properties of poly-9-vinyladenine. Biopolymers 9, 965-977 (1970).

38. Puri, M. C. M. C., et al., Interaction of the TEK and TIE receptor tyrosine kinases during cardiovascular development. Development 126, 4569-4580 (1999).

39. Rouleau, G. A. et al. Alteration in a new gene encoding a putative membrane-organizing protein causes neurofibromatosis type 2. Nature 363, 515-521 (1993).

40. Sambrook, J. et al. (1989). Molecular Cloning: A Laboratory Manual. Second Edition. Cold Spring Harbor Laboratory Press. New York.

41. Sato, N. et al. A gene family consisting of ezrin, radixin, and moesin. Its specific localization at actin filament/plasma membrane association sites. J. Cell Sci. 103, 131-143 (1992).

42. Shen, M. M., Identification of differentially expressed genes in mouse development using differential display and in situ hybridization. Methods 24, 15-27 (2001).

43. St.-Jaques, B., et al. A putative transmembrane protein with histidine-rich charge clusters encoded in the H-2K/t^{w5} region of mice. Mol. Cell Biol. 10, 138-145 (1990).

44. Stathakis, D. G., et al. The catecholamines up (Catsup) protein of Drosophila melanogaster functions as a negative regulator of tyrosine hydroxylase activity. Genetics 153, 361-382 (1999).

45. Summerton, J., et al., Morpholino antisense oligomers: design, preparation, and properties. Antisense Nucleic Acid Drug Dev. 7, 187-195 (1997).

46. Suzuki, A. and Takeshi, E. Ermelin, an endoplasmic reticulum transmembrane protein, contains the novel HELP domain conserved in eukaryotes. Gene 284, 31-40 (2002).

47. Suzuki, M. et al. A novel E2F binding protein with myc-type HLH motif stimulates E2F-dependent transcription by forming a heterodimer. Oncogene 17, 853-865 (1998).

48. Takeuchi, K. et al. Perturbation of cell adhesion and microvilli formation by antisense oligonucleotides to ERM family members. J. Cell Biol. 125, 1371-1384 (1994).

49. Tatjana, P. et al. Jaw and branchial arch mutants in zebrafish II: anterior arches and cartilage differentiation. Development 123, 345-356 (1996).

50. The Institute for Genomic Reseach. The TIGR Zebrafish Gene Index. ZGI Release 6.1 Mar. 15, 2001.

51. Thompson M. A., et al., The cloche and spadetail genes differentially affect hematopoiesis and vasculogenesis. Dev. Biol. 197, 248-269 (1998).

52. Thompson, J. D., et al. CLUSTAL W: improving the sensitivity of progressive multiple sequence alignment through sequence weighting, position-specific gap penalties and weight matrix choice. Nucleic Acids Res. 22, 4673-4680 (1994).

53. Trofatter, J. A. et al. A novel moesin-, ezrin-, radixin-like gene is a candidate for the neurofibromatosis 2 tumor suppressor. Cell 75, 826 (1993).

54. Turunen, O., et al. Cytovillin, a microvillar Mr 75,000 protein. cDNA sequence, prokaryotic expression and chromosomal localization. J. Biol. Chem. 264, 16727-16732 (1989).

55. Waterman, M. S. Introduction to Computation Biology: Maps, sequences and genomes. Chapman & Hall, London, pp.184 (1995).

56. Westerfield, M. The Zebrafish Book: A guide for the laboratory use of zebrafish (Danio rerio). 4^{th} ed., University of Oregon Press, Eugene (2000)

57. Wilgenbus, K. K., et al. Molecular cloning, cDNA sequence, and chromosomal assignment of the human radixin gene and two dispersed pseudogenes. Genomics 16, 199-206 (1993).

58. Yoshinori, D. et al. Normal development of mice and unimpaired cell adhesion/cell motility/actin-based cytoskeleton with compensatory up-regulation of ezrin or radixin in moesin gene knockout. J. Biol. Chem. 274, 2315-2321 (1999).

The embodiments set forth herein are intended to exemplify the invention and not to limit its scope or spirit. All of the patents, patent applications, journal articles, and publications set forth herein are hereby incorporated by reference in their entirety herein.

1. An isolated nucleic acid comprising a sequence that hybridizes under stringent conditions to a hybridization probe, wherein the probe is a member of the group consisting of SEQ ID NO 7, SEQ ID NO 16, and SEQ ID NO 34; or wherein the probe is a member of the group consisting of complements of SEQ ID NO 7, SEQ ID NO 16, and SEQ ID NO 34.

2. The nucleic acid sequence of claim 1 wherein the hybridization probe is SEQ ID NO 7 or a complement thereof.

3. The nucleic acid sequence of claim 1 wherein the hybridization probe is SEQ ID NO 16 or a complement thereof.

4. The nucleic acid sequence of claim 1 wherein the hybridization probe is SEQ ID NO 34 or a complement thereof.

5. The nucleic acid sequence of claim 1 wherein the hybridization probe is DNA, RNA, a nucleic acid analogue, or a combination of DNA and RNA.

6. The nucleic acid analogue sequence of claim 5 wherein the nucleic acid analogue sequence comprises phosphorothioate and morpholino phosphorodiamidate components.

7. The nucleic acid analogue sequence of claim 5 wherein the nucleic acid analogue sequence comprises peptide nucleic acid sequences.

8. The nucleic acid analogue sequence of claim 5 wherein the nucleic acid analogue sequence comprises locked nucleic acid sequences.

9. The nucleic acid sequence of claim 1 wherein the isolated nucleic acid sequence is at least 18 residues in length.

10. The nucleic acid sequence of claim 1 wherein the isolated nucleic acid sequence is from 15 to 100 residues in length.

11. The nucleic acid sequence of claim 1 wherein the isolated nucleic acid sequence comprises nonhybridizing portions that do not hybridize to the hybridization probe.

12. An isolated nucleic acid comprising a sequence that is at least 90% identical to a member of the group consisting of SEQ ID NO 7, SEQ ID NO 16, SEQ ID NO 34, and complements thereof.

13. The isolated nucleic acid of claim 12 wherein the isolated nucleic acid sequence is at least 90% identical to SEQ ID NO 7 and encodes a polypeptide that is a member of the TDE family, wherein the percent identity of the polypeptide sequence is closer to TDE1 than to other members of the TDE family.

14. The isolated nucleic acid of claim 12 wherein the isolated nucleic acid sequence is at least 90% identical to SEQ ID NO 16 and encodes a polypeptide that is a member of the PTV family, wherein the percent identity of the polypeptide sequence is closer to PTV than to other members of the PTV family.

15. The isolated nucleic acid of claim 12 wherein the isolated nucleic acid sequence is at least 90% identical to SEQ ID NO 34 and encodes a polypeptide that is a member of the HKE family, wherein the percent identity of the polypeptide sequence is closer to HKE4 than to other members of the HKE family

16. The isolated nucleic acid of claim 12 wherein the isolated nucleic acid sequence is at least 90% identical to SEQ ID NO 7 and encodes a polypeptide having vascular formation activity.

17. The isolated nucleic acid of claim 12 wherein the isolated nucleic acid sequence is at least 90% identical to SEQ ID NO 16 and encodes a polypeptide having blood vessel formation activity.

18. The isolated nucleic acid of claim 12 wherein the isolated nucleic acid sequence is at least 90% identical to SEQ ID NO 34 and encodes a polypeptide having cartilage forming or bone forming activity.

19. The isolated nucleic acid of claim 12 wherein
the isolated nucleic acid sequence comprises at least one change in the group consisting of point mutations, point deletions, polymorphisms, conservative substitutions, and degenerate substitutions when the isolated nucleic acid sequence is compared to a member of the group consisting of SEQ ID NO 7, SEQ ID NO 16 and SEQ ID NO 34.

20. A composition, the composition comprising:
an isolated polypeptide comprising an amino acid sequence that is at least 8 residues in length and is at least 90% identical to a member of the group consisting of SEQ ID NO 3, SEQ ID NO 6, SEQ ID NO 9, SEQ ID NO 12, SEQ ID NO 15, SEQ ID NO 18, SEQ ID NO 30, SEQ ID NO 33, and SEQ ID NO 36.

21. The composition of claim 20 wherein the amino acid sequence is at least 12 residues in length.

22. The composition of claim 20 wherein the amino acid sequence is at least 50 residues in length.

23. The composition of claim 20 wherein the amino acid sequence comprises at least one change in the group consisting of point mutations, point deletions, polymorphisms, conservative substitutions, and degenerate substitutions when the isolated nucleic acid sequence is compared to a member of the group consisting of SEQ ID NO 3, SEQ ID NO 6, SEQ ID NO 9, SEQ ID NO 12, SEQ ID NO 15, SEQ ID NO 18, SEQ ID NO 30, SEQ ID NO 33, and SEQ ID NO 36.

24. The composition of claim 20 wherein the amino acid sequence is at least 90% identical to SEQ ID NO 9 and the polypeptide has an activity for blood vessel formation.

25. The composition of claim 20 wherein the amino acid sequence is at least 90% identical to SEQ ID NO 27 and the polypeptide has an activity for blood vessel formation.

26. The composition of claim 20 wherein the amino acid sequence is at least 90% identical to SEQ ID NO 36 and the polypeptide has an activity for cartilage formation or bone formation.

27. The composition of claim 20 wherein the amino acid sequence is at least 95% identical to a member of the group consisting of SEQ ID NO 3, SEQ ID NO 6, SEQ ID NO 9, SEQ ID NO 12, SEQ ID NO 15, SEQ ID NO 18, SEQ ID NO 30, SEQ ID NO 33, SEQ ID NO 36, SEQ ID NO 39, SEQ ID NO 42, and SEQ ID NO 45.

28. The composition of claim 20 wherein the amino acid sequence
is a member of the group consisting of an amino acid sequences that are at least 90% identical to SEQ ID NO 9 and the polypeptide has an activity for blood vessel formation; at least 90% identical to SEQ ID NO 36 and the polypeptide has an activity for cartilage formation or bone formation; and
is at least 8 residues in length.

29. The composition of claim 20 further comprising a pharmaceutically acceptable buffer.

30. The composition of claim 20 wherein the polypeptide is a pharmaceutically acceptable salt.

31. An antisense polynucleic acid comprising a sequence, wherein the antisense polynucleic acid suppresses the expression of a polypeptide encoded by a polynucleic acid sequence for the polypeptide chosen from the group consisting of SEQ ID NO 1, SEQ ID NO 2, SEQ ID NO 4, SEQ ID NO 5, SEQ ID NO 7, SEQ ID NO 8, SEQ ID NO 10, SEQ ID NO 11, SEQ ID NO 13, SEQ ID NO 14, SEQ ID NO 16, SEQ ID NO 17, SEQ ID NO 19, SEQ ID NO 20, SEQ ID NO 22, SEQ ID NO 23, SEQ ID NO 25, SEQ ID NO 26, SEQ ID NO 28, SEQ ID NO 29, SEQ ID NO 31, SEQ ID NO 32, SEQ ID NO 34, and SEQ ID NO 35.

32. The antisense polynucleic acid of claim 31 wherein the antisense polynucleic acid has a backbone chosen from the group consisting of phosphorothioate, morpholino, and peptide linkage molecules.

33. The antisense polynucleic acid of claim 31 wherein the antisense polynucleic acid suppresses the expression of a polypeptide encoded by a polynucleic acid sequence for the polypeptide chosen from the group consisting of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, and SEQ ID NO 34 and antisense polynucleic acid is a directed to a noncoding portion of the polynucleic acid sequence for the polypeptide.

34. The antisense oligonucleotide of claim 30 wherein the antisense polynucleic acid has a number of residues that is at least 10.

35. The antisense oligonucleotide of claim 30 wherein the antisense polynucleic acid has a number of residues that ranges from 12 to 30.

36. A teleost comprising the antisense polynucleic acid of claim 30.

37. A Danio rerio comprising the antisense polynucleic acid of claim 30.

38. A cell comprising the antisense polynucleic acid of claim 30.

39. A cell comprising the antisense polynucleic acid of claim 34.

40. A vector, the vector comprising:
a first nucleic acid sequence that hybridizes under stringent conditions to a second nucleic acid sequence, wherein the second sequence is a member of the group consisting of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, or wherein the second sequence is a member of the group consisting of complements of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34;
wherein the first nucleic acid sequence is operably linked to an expression control sequence that directs production of a transcript from the first nucleic acid sequence.

41. The vector of claim 40 wherein the second nucleic acid sequence is SEQ ID NO 7 or a complement thereof.

42. The vector of claim 40 wherein second nucleic acid sequence is SEQ ID NO 16 or a complement thereof.

43. The vector of claim 40 wherein the second nucleic acid sequence is SEQ ID NO 25 or a complement thereof.

44. The vector of claim 40 wherein the second nucleic acid sequence is SEQ ID NO 34 or a complement thereof.

45. The vector of claim 40 wherein a number of residues in the first nucleic acid sequence is from 8 to 50.

46. The vector of claim 40 wherein the vector is a non-integrating vector.

47. The vector of claim 40 wherein the vector is an integrating vector.

48. The vector of claim 40 wherein the vector is an integrating non-viral vector.

49. The vector of claim 48 wherein the vector is a transposon vector.

50. The vector of claim 48 wherein the vector is a Sleeping Beauty transposon vector.

51. The vector of claim 40 wherein the vector is a member of the group consisting of lentiviruses and adenoviruses.

52. A vertebrate nonhuman animal comprising the vector of claim 49.

53. The vertebrate animal of claim 52 wherein the animal is a zebrafish.

54. The vertebrate animal of claim 52 wherein the animal is a mouse or a rat.

55. A method of using a composition, the method comprising administering a composition to an animal, the composition comprising a polypeptide having an amino acid sequence that is at least 90% identical to a member of the group consisting of SEQ ID NO 3, SEQ ID NO 6, SEQ ID NO 9, SEQ ID NO 12, SEQ ID NO 15, SEQ ID NO 18, SEQ ID NO 30, SEQ ID NO 33, SEQ ID NO 36.

56. The method of claim 55 wherein the composition is administered by topical application.

57. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to a wound by topical application or by injection.

58. The method of claim 55 wherein the composition is administered by injection.

59. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to a tumor.

60. The method of claim 59 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to a tumor by injection of the composition into or near the tumor.

61. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to a heart by injection of the composition into or near the heart.

62. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to an ischemic heart by injection of the composition into or near the heart.

63. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to necrotic tissue by injection of the composition into or near the tissue.

64. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to an ulcer by injection of the composition into or near the ulcer.

65. The method of claim 55 wherein the polypeptide has an amino acid sequence that is at least 90%. identical to a member of the group consisting of SEQ ID NO 3, SEQ ID NO 6, SEQ ID NO 9, SEQ ID NO 12, SEQ ID NO 15, and SEQ ID NO 18, and the composition is administered to a venous ulcer by injection of the composition into or near the ulcer.

66. The method of claim 55 wherein the polypeptide has an amino acid sequence that is at least 90% identical to a member of the group consisting of SEQ ID NO 3, SEQ ID NO 6, SEQ ID NO 9, SEQ ID NO 12, SEQ ID NO 15, and SEQ ID NO 18, and the composition is administered to a diabetic ulcer by injection of the composition into or near the ulcer.

67. The method of claim 55 wherein the polypeptide has an amino acid sequence that is at least 90% identical to SEQ ID NO 36 and the composition is administered to bone by injection of the composition into or near the bone.

68. The method of claim 55 wherein the polypeptide has an amino acid sequence that is at least 90% identical to SEQ ID NO 36 and the composition is administered to bone by application of the composition into or near the bone in conjunction with a surgical procedure.

69. The method of claim 55 wherein the polypeptide has an amino acid sequence that is at least 90% identical to SEQ ID NO 36 and the composition is administered to cartilaginous tissue by application of the composition into or near the tissue.

70. A method, the method comprising:
administering a vector to an animal, the vector comprising: a first nucleic acid sequence that hybridizes under stringent conditions to a second nucleic acid sequence, wherein the second sequence is a member of the group consisting of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34; or wherein the second sequence is a member of the group consisting of complements of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, and SEQ ID NO 43.

71. A screening method, the method comprising:
providing a polypeptide having an amino acid sequence that is at least 90% identical to a member of the group consisting of SEQ ID NO 9, SEQ ID NO 27, and SEQ ID NO 36;
exposing the polypeptide to a factor; and
determining that the factor has a specific binding affinity for the polypeptide.

72. The method of claim 71 wherein the factor is provided in isolated form by using a separations process that separates the bound factor from the polypeptide.

73. The method of claim 71 wherein the factor is isolated prior to exposure of the factor to the polypeptide.

74. The method of claim 71 wherein the polypeptide amino acid sequence is at least 95% identical to a member of the group consisting of SEQ ID NO 9, SEQ ID NO 27, and SEQ ID NO 36.

75. The method of claim 71 wherein the polypeptide amino acid sequence is at least 99% identical to a member of the group consisting of SEQ ID NO 9, SEQ ID NO 27, SEQ ID NO 36, and SEQ ID NO 45.

76. The method of claim 71 further comprising exposing the polypeptide to a cellular lysate that contains the factor.

77. The method of claim 71 wherein the factor is a small molecule that binds the polypeptide.

78. A method of administering a compound, the method comprising preparing a composition of a factor, wherein the factor is isolated by the method of claim 71.

79. A composition, the composition comprising a combination of a pharmaceutically acceptable carrier, VEGF, and at least one member of the group consisting of TDE1 and PTV.

80. The composition of claim 79 wherein the VEGF and the at least one member of the group consisting of TDE1 and PTV are packaged as a kit.

81. A method of using a composition, the method comprising administering the composition to an animal, the composition comprising a polypeptide having an amino acid sequence that is at least 90% identical to SEQ ID NO 27.

82. The method of claim 80 wherein the composition is administered by a mechanism in the group consisting of topical application or injection.

83. The method of claim 81 wherein the composition is administered according to a member of the group consisting of injection of the composition into or near a tumor, injection of the composition into or near a heart, and injection of the composition into or near a necrotic tissue.

84. The method of claim 81 wherein the composition is administered into or near a diabetic or venous ulcer.
